SEQUENCE LISTING (468 sequences)

SEQ ID NO: 1 - DNA, 19 bp, Artificial Sequence
  based on Homo sapiens. Each nucleobase may be part of a ribonucleotide, deoxyribonucleotide, or nucleotide analog
aaaanncnaagnaccngca

SEQ ID NO: 2 - DNA, 19 bp, Artificial Sequence
  based on Homo sapiens. Each nucleobase may be part of a ribonucleotide, deoxyribonucleotide, or nucleotide analog
ncnagagggnggcncagga

SEQ ID NO: 3 - DNA, 19 bp, Artificial Sequence
  based on Homo sapiens. Each nucleobase may be part of a ribonucleotide, deoxyribonucleotide, or nucleotide analog
caganananangnaacacn

SEQ ID NO: 4 - DNA, 19 bp, Artificial Sequence
  based on Homo sapiens. Each nucleobase may be part of a ribonucleotide, deoxyribonucleotide, or nucleotide analog
ngagagcccnnnnnnngnn

SEQ ID NO: 5 - DNA, 19 bp, Artificial Sequence
  based on Homo sapiens. Each nucleobase may be part of a ribonucleotide, deoxyribonucleotide, or nucleotide analog
agnangaaanannncngan

SEQ ID NO: 6 - DNA, 19 bp, Artificial Sequence
  based on Homo sapiens. Each nucleobase may be part of a ribonucleotide, deoxyribonucleotide, or nucleotide analog
annggnnccaangngnncn

SEQ ID NO: 7 - DNA, 19 bp, Artificial Sequence
  based on Homo sapiens. Each nucleobase may be part of a ribonucleotide, deoxyribonucleotide, or nucleotide analog
nnagcaaaanangnnnnaa

SEQ ID NO: 8 - DNA, 19 bp, Artificial Sequence
  based on Homo sapiens. Each nucleobase may be part of a ribonucleotide, deoxyribonucleotide, or nucleotide analog
ngaannaannnnnaananc

SEQ ID NO: 9 - DNA, 19 bp, Artificial Sequence
  based on Homo sapiens. Each nucleobase may be part of a ribonucleotide, deoxyribonucleotide, or nucleotide analog
anncaaggcancaaagnng

SEQ ID NO: 10 - DNA, 19 bp, Artificial Sequence
  based on Homo sapiens. Each nucleobase may be part of a ribonucleotide, deoxyribonucleotide, or nucleotide analog
gncaaancannaannagga

SEQ ID NO: 11 - DNA, 19 bp, Artificial Sequence
  based on Homo sapiens. Each nucleobase may be part of a ribonucleotide, deoxyribonucleotide, or nucleotide analog
aanangnaaacngngangc

SEQ ID NO: 12 - DNA, 19 bp, Artificial Sequence
  based on Homo sapiens. Each nucleobase may be part of a ribonucleotide, deoxyribonucleotide, or nucleotide analog
gcagaanaaaacnaanaan

SEQ ID NO: 13 - DNA, 19 bp, Artificial Sequence
  based on Homo sapiens. Each nucleobase may be part of a ribonucleotide, deoxyribonucleotide, or nucleotide analog
gaaagnaanannnaagcag

SEQ ID NO: 14 - DNA, 19 bp, Artificial Sequence
  based on Homo sapiens. Each nucleobase may be part of a ribonucleotide, deoxyribonucleotide, or nucleotide analog
nnaccacancanncaagnc

SEQ ID NO: 15 - DNA, 19 bp, Artificial Sequence
  based on Homo sapiens. Each nucleobase may be part of a ribonucleotide, deoxyribonucleotide, or nucleotide analog
cnaaanacnagagnncgac

SEQ ID NO: 16 - DNA, 19 bp, Artificial Sequence
  based on Homo sapiens. Each nucleobase may be part of a ribonucleotide, deoxyribonucleotide, or nucleotide analog
acacgaccgcnaagaaaca

SEQ ID NO: 17 - DNA, 19 bp, Artificial Sequence
  based on Homo sapiens. Each nucleobase may be part of a ribonucleotide, deoxyribonucleotide, or nucleotide analog
nanccacnnangacanaaa

SEQ ID NO: 18 - DNA, 19 bp, Artificial Sequence
  based on Homo sapiens. Each nucleobase may be part of a ribonucleotide, deoxyribonucleotide, or nucleotide analog
gnnanaggagcnaacaaan

SEQ ID NO: 19 - DNA, 19 bp, Artificial Sequence
  based on Homo sapiens. Each nucleobase may be part of a ribonucleotide, deoxyribonucleotide, or nucleotide analog
aangngaaacacaagcaac

SEQ ID NO: 20 - DNA, 19 bp, Artificial Sequence
  based on Homo sapiens. Each nucleobase may be part of a ribonucleotide, deoxyribonucleotide, or nucleotide analog
acannanannaggaaancc

SEQ ID NO: 21 - DNA, 19 bp, Artificial Sequence
  based on Homo sapiens. Each nucleobase may be part of a ribonucleotide, deoxyribonucleotide, or nucleotide analog
cnngnccaccnnnncnaaa

SEQ ID NO: 22 - DNA, 19 bp, Artificial Sequence
  based on Homo sapiens. Each nucleobase may be part of a ribonucleotide, deoxyribonucleotide, or nucleotide analog
ancnncncnngaaaanagg

SEQ ID NO: 23 - DNA, 19 bp, Artificial Sequence
  based on Homo sapiens. Each nucleobase may be part of a ribonucleotide, deoxyribonucleotide, or nucleotide analog
ccnncaaaacngnnaaaag

SEQ ID NO: 24 - DNA, 19 bp, Artificial Sequence
  based on Homo sapiens. Each nucleobase may be part of a ribonucleotide, deoxyribonucleotide, or nucleotide analog
angncngcaggnacacaag

SEQ ID NO: 25 - DNA, 19 bp, Artificial Sequence
  based on Homo sapiens. Each nucleobase may be part of a ribonucleotide, deoxyribonucleotide, or nucleotide analog
ancnannaaacncnncnac

SEQ ID NO: 26 - DNA, 19 bp, Artificial Sequence
  based on Homo sapiens. Each nucleobase may be part of a ribonucleotide, deoxyribonucleotide, or nucleotide analog
acaggacnaccacnnggaa

SEQ ID NO: 27 - DNA, 19 bp, Artificial Sequence
  based on Homo sapiens. Each nucleobase may be part of a ribonucleotide, deoxyribonucleotide, or nucleotide analog
ngccagngnngangcngaa

SEQ ID NO: 28 - DNA, 19 bp, Artificial Sequence
  based on Homo sapiens. Each nucleobase may be part of a ribonucleotide, deoxyribonucleotide, or nucleotide analog
gnanaaagaaacccngcnc

SEQ ID NO: 29 - DNA, 19 bp, Artificial Sequence
  based on Homo sapiens. Each nucleobase may be part of a ribonucleotide, deoxyribonucleotide, or nucleotide analog
cgcacggnancnccnncac

SEQ ID NO: 30 - DNA, 19 bp, Artificial Sequence
  based on Homo sapiens. Each nucleobase may be part of a ribonucleotide, deoxyribonucleotide, or nucleotide analog
cnacagcngcangacaacn

SEQ ID NO: 31 - DNA, 19 bp, Artificial Sequence
  based on Homo sapiens. Each nucleobase may be part of a ribonucleotide, deoxyribonucleotide, or nucleotide analog
gcngagncnccananngcc

SEQ ID NO: 32 - DNA, 19 bp, Artificial Sequence
  based on Homo sapiens. Each nucleobase may be part of a ribonucleotide, deoxyribonucleotide, or nucleotide analog
anacnnnccngngncnncc

SEQ ID NO: 33 - DNA, 19 bp, Artificial Sequence
  based on Homo sapiens. Each nucleobase may be part of a ribonucleotide, deoxyribonucleotide, or nucleotide analog
ganaaancngcaannnggg

SEQ ID NO: 34 - DNA, 19 bp, Artificial Sequence
  based on Homo sapiens. Each nucleobase may be part of a ribonucleotide, deoxyribonucleotide, or nucleotide analog
nngnagacngcgnggcacn

SEQ ID NO: 35 - DNA, 19 bp, Artificial Sequence
  based on Homo sapiens. Each nucleobase may be part of a ribonucleotide, deoxyribonucleotide, or nucleotide analog
accanncngganaccagaa

SEQ ID NO: 36 - DNA, 19 bp, Artificial Sequence
  based on Homo sapiens. Each nucleobase may be part of a ribonucleotide, deoxyribonucleotide, or nucleotide analog
agnnnncaacnnngnacng

SEQ ID NO: 37 - DNA, 19 bp, Artificial Sequence
  based on Homo sapiens. Each nucleobase may be part of a ribonucleotide, deoxyribonucleotide, or nucleotide analog
angancncngcnncccaga

SEQ ID NO: 38 - DNA, 19 bp, Artificial Sequence
  based on Homo sapiens. Each nucleobase may be part of a ribonucleotide, deoxyribonucleotide, or nucleotide analog
aganggccngncnaaggca

SEQ ID NO: 39 - DNA, 19 bp, Artificial Sequence
  based on Homo sapiens. Each nucleobase may be part of a ribonucleotide, deoxyribonucleotide, or nucleotide analog
agnncncaaaaganagncn

SEQ ID NO: 40 - DNA, 19 bp, Artificial Sequence
  based on Homo sapiens. Each nucleobase may be part of a ribonucleotide, deoxyribonucleotide, or nucleotide analog
gngncngananancnacaa

SEQ ID NO: 41 - DNA, 19 bp, Artificial Sequence
  based on Homo sapiens. Each nucleobase may be part of a ribonucleotide, deoxyribonucleotide, or nucleotide analog
ncgggnananggngncnga

SEQ ID NO: 42 - DNA, 19 bp, Artificial Sequence
  based on Homo sapiens. Each nucleobase may be part of a ribonucleotide, deoxyribonucleotide, or nucleotide analog
cagggnnccncgggnanan

SEQ ID NO: 43 - DNA, 19 bp, Artificial Sequence
  based on Homo sapiens. Each nucleobase may be part of a ribonucleotide, deoxyribonucleotide, or nucleotide analog
gcnncnncacaanacangg

SEQ ID NO: 44 - DNA, 19 bp, Artificial Sequence
  based on Homo sapiens. Each nucleobase may be part of a ribonucleotide, deoxyribonucleotide, or nucleotide analog
ggccagnncngaaaggacn

SEQ ID NO: 45 - DNA, 19 bp, Artificial Sequence
  based on Homo sapiens. Each nucleobase may be part of a ribonucleotide, deoxyribonucleotide, or nucleotide analog
gcnaacncncnnggggnna

SEQ ID NO: 46 - DNA, 19 bp, Artificial Sequence
  based on Homo sapiens. Each nucleobase may be part of a ribonucleotide, deoxyribonucleotide, or nucleotide analog
gngnagnagagnccagcac

SEQ ID NO: 47 - DNA, 19 bp, Artificial Sequence
  based on Homo sapiens. Each nucleobase may be part of a ribonucleotide, deoxyribonucleotide, or nucleotide analog
aagcacngcacnnggncac

SEQ ID NO: 48 - DNA, 19 bp, Artificial Sequence
  based on Homo sapiens. Each nucleobase may be part of a ribonucleotide, deoxyribonucleotide, or nucleotide analog
nncagnnnnccaccacaac

SEQ ID NO: 49 - DNA, 19 bp, Artificial Sequence
  based on Homo sapiens. Each nucleobase may be part of a ribonucleotide, deoxyribonucleotide, or nucleotide analog
acgancacaaggnncccaa

SEQ ID NO: 50 - DNA, 19 bp, Artificial Sequence
  based on Homo sapiens. Each nucleobase may be part of a ribonucleotide, deoxyribonucleotide, or nucleotide analog
ncgccngngnncngaccag

SEQ ID NO: 51 - DNA, 19 bp, Artificial Sequence
  based on Homo sapiens. Each nucleobase may be part of a ribonucleotide, deoxyribonucleotide, or nucleotide analog
ccggcccaaaacaaagaag

SEQ ID NO: 52 - DNA, 19 bp, Artificial Sequence
  based on Homo sapiens. Each nucleobase may be part of a ribonucleotide, deoxyribonucleotide, or nucleotide analog
ganncacnncgaanannaa

SEQ ID NO: 53 - DNA, 19 bp, Artificial Sequence
  based on Homo sapiens. Each nucleobase may be part of a ribonucleotide, deoxyribonucleotide, or nucleotide analog
nancagaacncacagcanc

SEQ ID NO: 54 - DNA, 19 bp, Artificial Sequence
  based on Homo sapiens. Each nucleobase may be part of a ribonucleotide, deoxyribonucleotide, or nucleotide analog
ggaagannngnngaannng

SEQ ID NO: 55 - DNA, 19 bp, Artificial Sequence
  based on Homo sapiens. Each nucleobase may be part of a ribonucleotide, deoxyribonucleotide, or nucleotide analog
ncngccangganggannnc

SEQ ID NO: 56 - DNA, 19 bp, Artificial Sequence
  based on Homo sapiens. Each nucleobase may be part of a ribonucleotide, deoxyribonucleotide, or nucleotide analog
aagnaaaganccgngcnnc

SEQ ID NO: 57 - DNA, 19 bp, Artificial Sequence
  based on Homo sapiens. Each nucleobase may be part of a ribonucleotide, deoxyribonucleotide, or nucleotide analog
cngagnananccangnccc

SEQ ID NO: 58 - DNA, 19 bp, Artificial Sequence
  based on Homo sapiens. Each nucleobase may be part of a ribonucleotide, deoxyribonucleotide, or nucleotide analog
gcaagcngcnccnngnnaa

SEQ ID NO: 59 - DNA, 19 bp, Artificial Sequence
  based on Homo sapiens. Each nucleobase may be part of a ribonucleotide, deoxyribonucleotide, or nucleotide analog
aaagcanaaaanccagcnc

SEQ ID NO: 60 - DNA, 19 bp, Artificial Sequence
  based on Homo sapiens. Each nucleobase may be part of a ribonucleotide, deoxyribonucleotide, or nucleotide analog
gaaagcacnnnacnnnanc

SEQ ID NO: 61 - DNA, 19 bp, Artificial Sequence
  based on Homo sapiens. Each nucleobase may be part of a ribonucleotide, deoxyribonucleotide, or nucleotide analog
acngggcnnccaancagnn

SEQ ID NO: 62 - DNA, 19 bp, Artificial Sequence
  based on Homo sapiens. Each nucleobase may be part of a ribonucleotide, deoxyribonucleotide, or nucleotide analog
gnngnncccaagggncnnc

SEQ ID NO: 63 - DNA, 19 bp, Artificial Sequence
  based on Homo sapiens. Each nucleobase may be part of a ribonucleotide, deoxyribonucleotide, or nucleotide analog
acccngganaccannnagc

SEQ ID NO: 64 - DNA, 19 bp, Artificial Sequence
  based on Homo sapiens. Each nucleobase may be part of a ribonucleotide, deoxyribonucleotide, or nucleotide analog
ngnncnaacaganannngc

SEQ ID NO: 65 - DNA, 19 bp, Artificial Sequence
  based on Homo sapiens. Each nucleobase may be part of a ribonucleotide, deoxyribonucleotide, or nucleotide analog
nanananncnngncccnnc

SEQ ID NO: 66 - DNA, 19 bp, Artificial Sequence
  based on Homo sapiens. Each nucleobase may be part of a ribonucleotide, deoxyribonucleotide, or nucleotide analog
agnnaaangaananngnnn

SEQ ID NO: 67 - DNA, 19 bp, Artificial Sequence
  based on Homo sapiens. Each nucleobase may be part of a ribonucleotide, deoxyribonucleotide, or nucleotide analog
gacacnccncaagngaang

SEQ ID NO: 68 - DNA, 19 bp, Artificial Sequence
  based on Homo sapiens. Each nucleobase may be part of a ribonucleotide, deoxyribonucleotide, or nucleotide analog
nnncncagnagnncnnacc

SEQ ID NO: 69 - DNA, 19 bp, Artificial Sequence
  based on Homo sapiens. Each nucleobase may be part of a ribonucleotide, deoxyribonucleotide, or nucleotide analog
gnnagnganggngnnnncn

SEQ ID NO: 70 - DNA, 19 bp, Artificial Sequence
  based on Homo sapiens. Each nucleobase may be part of a ribonucleotide, deoxyribonucleotide, or nucleotide analog
aganggnancancaanncn

SEQ ID NO: 71 - DNA, 19 bp, Artificial Sequence
  based on Homo sapiens. Each nucleobase may be part of a ribonucleotide, deoxyribonucleotide, or nucleotide analog
ngnaccanaggannnngga

SEQ ID NO: 72 - DNA, 19 bp, Artificial Sequence
  based on Homo sapiens. Each nucleobase may be part of a ribonucleotide, deoxyribonucleotide, or nucleotide analog
ccccanncgnanagcnncn

SEQ ID NO: 73 - DNA, 19 bp, Artificial Sequence
  based on Homo sapiens. Each nucleobase may be part of a ribonucleotide, deoxyribonucleotide, or nucleotide analog
annannnncnnaangnccn

SEQ ID NO: 74 - DNA, 19 bp, Artificial Sequence
  based on Homo sapiens. Each nucleobase may be part of a ribonucleotide, deoxyribonucleotide, or nucleotide analog
caagngannnanagnngcn

SEQ ID NO: 75 - DNA, 19 bp, Artificial Sequence
  based on Homo sapiens. Each nucleobase may be part of a ribonucleotide, deoxyribonucleotide, or nucleotide analog
nagancngcaaccagaacc

SEQ ID NO: 76 - DNA, 19 bp, Artificial Sequence
  based on Homo sapiens. Each nucleobase may be part of a ribonucleotide, deoxyribonucleotide, or nucleotide analog
cancnngcanacngncnnn

SEQ ID NO: 77 - DNA, 19 bp, Artificial Sequence
  based on Homo sapiens. Each nucleobase may be part of a ribonucleotide, deoxyribonucleotide, or nucleotide analog
ccnnagcngcncnncagna

SEQ ID NO: 78 - DNA, 19 bp, Artificial Sequence
  based on Homo sapiens. Each nucleobase may be part of a ribonucleotide, deoxyribonucleotide, or nucleotide analog
aagcnncnccncnngcagg

SEQ ID NO: 79 - DNA, 19 bp, Artificial Sequence
  based on Homo sapiens. Each nucleobase may be part of a ribonucleotide, deoxyribonucleotide, or nucleotide analog
anannncnanccanacaga

SEQ ID NO: 80 - DNA, 19 bp, Artificial Sequence
  based on Homo sapiens. Each nucleobase may be part of a ribonucleotide, deoxyribonucleotide, or nucleotide analog
cnagangnccacaaggaac

SEQ ID NO: 81 - DNA, 19 bp, Artificial Sequence
  based on Homo sapiens. Each nucleobase may be part of a ribonucleotide, deoxyribonucleotide, or nucleotide analog
agcacanngnnnacaagng

SEQ ID NO: 82 - DNA, 19 bp, Artificial Sequence
  based on Homo sapiens. Each nucleobase may be part of a ribonucleotide, deoxyribonucleotide, or nucleotide analog
agcacangggacacnngnc

SEQ ID NO: 83 - DNA, 19 bp, Artificial Sequence
  based on Homo sapiens. Each nucleobase may be part of a ribonucleotide, deoxyribonucleotide, or nucleotide analog
cnngaaagnaangacngng

SEQ ID NO: 84 - DNA, 19 bp, Artificial Sequence
  based on Homo sapiens. Each nucleobase may be part of a ribonucleotide, deoxyribonucleotide, or nucleotide analog
ccnacnanagagnnagann

SEQ ID NO: 85 - DNA, 19 bp, Artificial Sequence
  based on Homo sapiens. Each nucleobase may be part of a ribonucleotide, deoxyribonucleotide, or nucleotide analog
anncaancagggnaanaag

SEQ ID NO: 86 - DNA, 19 bp, Artificial Sequence
  based on Homo sapiens. Each nucleobase may be part of a ribonucleotide, deoxyribonucleotide, or nucleotide analog
aagncagnncacancacac

SEQ ID NO: 87 - DNA, 19 bp, Artificial Sequence
  based on Homo sapiens. Each nucleobase may be part of a ribonucleotide, deoxyribonucleotide, or nucleotide analog
cagnaaaaaaaangganaa

SEQ ID NO: 88 - DNA, 19 bp, Artificial Sequence
  based on Homo sapiens. Each nucleobase may be part of a ribonucleotide, deoxyribonucleotide, or nucleotide analog
nncagnnanagnangangc

SEQ ID NO: 89 - DNA, 19 bp, Artificial Sequence
  based on Homo sapiens. Each nucleobase may be part of a ribonucleotide, deoxyribonucleotide, or nucleotide analog
nacacnnagaaannaaanc

SEQ ID NO: 90 - DNA, 19 bp, Artificial Sequence
  based on Homo sapiens. Each nucleobase may be part of a ribonucleotide, deoxyribonucleotide, or nucleotide analog
ncncnancnnnccaccagc

SEQ ID NO: 91 - DNA, 19 bp, Artificial Sequence
  based on Homo sapiens. Each nucleobase may be part of a ribonucleotide, deoxyribonucleotide, or nucleotide analog
agaanccnaaaacacaaca

SEQ ID NO: 92 - DNA, 19 bp, Artificial Sequence
  based on Homo sapiens. Each nucleobase may be part of a ribonucleotide, deoxyribonucleotide, or nucleotide analog
anncgcacaagnacgngnn

SEQ ID NO: 93 - DNA, 19 bp, Artificial Sequence
  based on Homo sapiens. Each nucleobase may be part of a ribonucleotide, deoxyribonucleotide, or nucleotide analog
ngncagnacangnnggcnc

SEQ ID NO: 94 - DNA, 19 bp, Artificial Sequence
  based on Homo sapiens. Each nucleobase may be part of a ribonucleotide, deoxyribonucleotide, or nucleotide analog
acanagngnnnngccacnn

SEQ ID NO: 95 - DNA, 19 bp, Artificial Sequence
  based on Homo sapiens. Each nucleobase may be part of a ribonucleotide, deoxyribonucleotide, or nucleotide analog
cnnngancnggcncagacn

SEQ ID NO: 96 - DNA, 19 bp, Artificial Sequence
  based on Homo sapiens. Each nucleobase may be part of a ribonucleotide, deoxyribonucleotide, or nucleotide analog
gaaaccacannnaacagnn

SEQ ID NO: 97 - DNA, 19 bp, Artificial Sequence
  based on Homo sapiens. Each nucleobase may be part of a ribonucleotide, deoxyribonucleotide, or nucleotide analog
ncannngagccngggaggn

SEQ ID NO: 98 - DNA, 19 bp, Artificial Sequence
  based on Homo sapiens. Each nucleobase may be part of a ribonucleotide, deoxyribonucleotide, or nucleotide analog
cggaggcngaggcaggaga

SEQ ID NO: 99 - DNA, 19 bp, Artificial Sequence
  based on Homo sapiens. Each nucleobase may be part of a ribonucleotide, deoxyribonucleotide, or nucleotide analog
ggngnggnggnacgcgccn

SEQ ID NO: 100 - DNA, 19 bp, Artificial Sequence
  based on Homo sapiens. Each nucleobase may be part of a ribonucleotide, deoxyribonucleotide, or nucleotide analog
acccangcacaaaacnacc

SEQ ID NO: 101 - DNA, 19 bp, Artificial Sequence
  based on Homo sapiens. Each nucleobase may be part of a ribonucleotide, deoxyribonucleotide, or nucleotide analog
agaangngccagnaggaga

SEQ ID NO: 102 - DNA, 19 bp, Artificial Sequence
  based on Homo sapiens. Each nucleobase may be part of a ribonucleotide, deoxyribonucleotide, or nucleotide analog
ncncacagacgnngggcnn

SEQ ID NO: 103 - DNA, 19 bp, Artificial Sequence
  based on Homo sapiens. Each nucleobase may be part of a ribonucleotide, deoxyribonucleotide, or nucleotide analog
ccagnggnnngcaagcang

SEQ ID NO: 104 - DNA, 19 bp, Artificial Sequence
  based on Homo sapiens. Each nucleobase may be part of a ribonucleotide, deoxyribonucleotide, or nucleotide analog
gaaannnagnggccaggaa

SEQ ID NO: 105 - DNA, 19 bp, Artificial Sequence
  based on Homo sapiens. Each nucleobase may be part of a ribonucleotide, deoxyribonucleotide, or nucleotide analog
agaaanacacaanngcacc

SEQ ID NO: 106 - DNA, 19 bp, Artificial Sequence
  based on Homo sapiens. Each nucleobase may be part of a ribonucleotide, deoxyribonucleotide, or nucleotide analog
nacnganacannnnaagga

SEQ ID NO: 107 - DNA, 19 bp, Artificial Sequence
  based on Homo sapiens. Each nucleobase may be part of a ribonucleotide, deoxyribonucleotide, or nucleotide analog
nncaacanggaganncnaa

SEQ ID NO: 108 - DNA, 19 bp, Artificial Sequence
  based on Homo sapiens. Each nucleobase may be part of a ribonucleotide, deoxyribonucleotide, or nucleotide analog
annncnangcannnagagn

SEQ ID NO: 109 - DNA, 19 bp, Artificial Sequence
  based on Homo sapiens. Each nucleobase may be part of a ribonucleotide, deoxyribonucleotide, or nucleotide analog
aanacnaggcngaaaagcc

SEQ ID NO: 110 - DNA, 19 bp, Artificial Sequence
  based on Homo sapiens. Each nucleobase may be part of a ribonucleotide, deoxyribonucleotide, or nucleotide analog
ggcnnngcnnnnancagnn

SEQ ID NO: 111 - DNA, 19 bp, Artificial Sequence
  based on Homo sapiens. Each nucleobase may be part of a ribonucleotide, deoxyribonucleotide, or nucleotide analog
ncnagggaggnagnnnngn

SEQ ID NO: 112 - DNA, 19 bp, Artificial Sequence
  based on Homo sapiens. Each nucleobase may be part of a ribonucleotide, deoxyribonucleotide, or nucleotide analog
gggaagaaaagggacnagc

SEQ ID NO: 113 - DNA, 19 bp, Artificial Sequence
  based on Homo sapiens. Each nucleobase may be part of a ribonucleotide, deoxyribonucleotide, or nucleotide analog
gnncanaangaaangaang

SEQ ID NO: 114 - DNA, 19 bp, Artificial Sequence
  based on Homo sapiens. Each nucleobase may be part of a ribonucleotide, deoxyribonucleotide, or nucleotide analog
anaagaanangcngnnnnc

SEQ ID NO: 115 - DNA, 19 bp, Artificial Sequence
  based on Homo sapiens. Each nucleobase may be part of a ribonucleotide, deoxyribonucleotide, or nucleotide analog
nncaaacgngnnggcgcnn

SEQ ID NO: 116 - DNA, 19 bp, Artificial Sequence
  based on Homo sapiens. Each nucleobase may be part of a ribonucleotide, deoxyribonucleotide, or nucleotide analog
angacaagncgnannncag

SEQ ID NO: 117 - DNA, 19 bp, Artificial Sequence
  based on Homo sapiens. Each nucleobase may be part of a ribonucleotide, deoxyribonucleotide, or nucleotide analog
aagnggaanacgnagacan

SEQ ID NO: 118 - DNA, 19 bp, Artificial Sequence
  based on Homo sapiens. Each nucleobase may be part of a ribonucleotide, deoxyribonucleotide, or nucleotide analog
agacaggaaccccagcagg

SEQ ID NO: 119 - DNA, 19 bp, Artificial Sequence
  based on Homo sapiens. Each nucleobase may be part of a ribonucleotide, deoxyribonucleotide, or nucleotide analog
cgagcaagacnccnnncng

SEQ ID NO: 120 - DNA, 19 bp, Artificial Sequence
  based on Homo sapiens. Each nucleobase may be part of a ribonucleotide, deoxyribonucleotide, or nucleotide analog
agngnaanagaaaccagca

SEQ ID NO: 121 - DNA, 19 bp, Artificial Sequence
  based on Homo sapiens. Each nucleobase may be part of a ribonucleotide, deoxyribonucleotide, or nucleotide analog
ngaccnngncanncacacc

SEQ ID NO: 122 - DNA, 19 bp, Artificial Sequence
  based on Homo sapiens. Each nucleobase may be part of a ribonucleotide, deoxyribonucleotide, or nucleotide analog
nnanccagcancaggccac

SEQ ID NO: 123 - DNA, 19 bp, Artificial Sequence
  based on Homo sapiens. Each nucleobase may be part of a ribonucleotide, deoxyribonucleotide, or nucleotide analog
acngncnccncnnnnccag

SEQ ID NO: 124 - DNA, 19 bp, Artificial Sequence
  based on Homo sapiens. Each nucleobase may be part of a ribonucleotide, deoxyribonucleotide, or nucleotide analog
nnnnangcnnnncagnagg

SEQ ID NO: 125 - DNA, 19 bp, Artificial Sequence
  based on Homo sapiens. Each nucleobase may be part of a ribonucleotide, deoxyribonucleotide, or nucleotide analog
acgaancngcagcnaggan

SEQ ID NO: 126 - DNA, 19 bp, Artificial Sequence
  based on Homo sapiens. Each nucleobase may be part of a ribonucleotide, deoxyribonucleotide, or nucleotide analog
caagnngnnaacggaannn

SEQ ID NO: 127 - DNA, 19 bp, Artificial Sequence
  based on Homo sapiens. Each nucleobase may be part of a ribonucleotide, deoxyribonucleotide, or nucleotide analog
naggcngagaggnagcnnc

SEQ ID NO: 128 - DNA, 19 bp, Artificial Sequence
  based on Homo sapiens. Each nucleobase may be part of a ribonucleotide, deoxyribonucleotide, or nucleotide analog
gnnacngaagaaggaaaag

SEQ ID NO: 129 - DNA, 19 bp, Artificial Sequence
  based on Homo sapiens. Each nucleobase may be part of a ribonucleotide, deoxyribonucleotide, or nucleotide analog
gaangagngngnggaangn

SEQ ID NO: 130 - DNA, 19 bp, Artificial Sequence
  based on Homo sapiens. Each nucleobase may be part of a ribonucleotide, deoxyribonucleotide, or nucleotide analog
ngnnnncngnacccggaag

SEQ ID NO: 131 - DNA, 19 bp, Artificial Sequence
  based on Homo sapiens. Each nucleobase may be part of a ribonucleotide, deoxyribonucleotide, or nucleotide analog
gagccacggaaananccac

SEQ ID NO: 132 - DNA, 19 bp, Artificial Sequence
  based on Homo sapiens. Each nucleobase may be part of a ribonucleotide, deoxyribonucleotide, or nucleotide analog
nganggagagnnngaanaa

SEQ ID NO: 133 - DNA, 19 bp, Artificial Sequence
  based on Homo sapiens. Each nucleobase may be part of a ribonucleotide, deoxyribonucleotide, or nucleotide analog
gannngcncnggagnnnac

SEQ ID NO: 134 - DNA, 19 bp, Artificial Sequence
  based on Homo sapiens. Each nucleobase may be part of a ribonucleotide, deoxyribonucleotide, or nucleotide analog
ggcagaaaanncnngannn

SEQ ID NO: 135 - DNA, 19 bp, Artificial Sequence
  based on Homo sapiens. Each nucleobase may be part of a ribonucleotide, deoxyribonucleotide, or nucleotide analog
ggacaggggnaggaacnnc

SEQ ID NO: 136 - DNA, 19 bp, Artificial Sequence
  based on Homo sapiens. Each nucleobase may be part of a ribonucleotide, deoxyribonucleotide, or nucleotide analog
gcannnncgnnanncanng

SEQ ID NO: 137 - DNA, 19 bp, Artificial Sequence
  based on Homo sapiens. Each nucleobase may be part of a ribonucleotide, deoxyribonucleotide, or nucleotide analog
cngaaaagnaagnaancng

SEQ ID NO: 138 - DNA, 19 bp, Artificial Sequence
  based on Homo sapiens. Each nucleobase may be part of a ribonucleotide, deoxyribonucleotide, or nucleotide analog
ggcgacagaaaagncaang

SEQ ID NO: 139 - DNA, 19 bp, Artificial Sequence
  based on Homo sapiens. Each nucleobase may be part of a ribonucleotide, deoxyribonucleotide, or nucleotide analog
ccacncngncnccaggncc

SEQ ID NO: 140 - DNA, 19 bp, Artificial Sequence
  based on Homo sapiens. Each nucleobase may be part of a ribonucleotide, deoxyribonucleotide, or nucleotide analog
ccaccacaggcaaagcaag

SEQ ID NO: 141 - DNA, 19 bp, Artificial Sequence
  based on Homo sapiens. Each nucleobase may be part of a ribonucleotide, deoxyribonucleotide, or nucleotide analog
nncggnncccaanngcnca

SEQ ID NO: 142 - DNA, 19 bp, Artificial Sequence
  based on Homo sapiens. Each nucleobase may be part of a ribonucleotide, deoxyribonucleotide, or nucleotide analog
nncngacanagcannancc

SEQ ID NO: 143 - DNA, 19 bp, Artificial Sequence
  based on Homo sapiens. Each nucleobase may be part of a ribonucleotide, deoxyribonucleotide, or nucleotide analog
ngggaaaangncncaggng

SEQ ID NO: 144 - DNA, 19 bp, Artificial Sequence
  based on Homo sapiens. Each nucleobase may be part of a ribonucleotide, deoxyribonucleotide, or nucleotide analog
nanaaangggcannnggga

SEQ ID NO: 145 - DNA, 19 bp, Artificial Sequence
  based on Homo sapiens. Each nucleobase may be part of a ribonucleotide, deoxyribonucleotide, or nucleotide analog
ngncnngaagcngannnnc

SEQ ID NO: 146 - DNA, 19 bp, Artificial Sequence
  based on Homo sapiens. Each nucleobase may be part of a ribonucleotide, deoxyribonucleotide, or nucleotide analog
gaaacngngnancnngaag

SEQ ID NO: 147 - DNA, 19 bp, Artificial Sequence
  based on Homo sapiens. Each nucleobase may be part of a ribonucleotide, deoxyribonucleotide, or nucleotide analog
ngncngcangcncaganna

SEQ ID NO: 148 - DNA, 19 bp, Artificial Sequence
  based on Homo sapiens. Each nucleobase may be part of a ribonucleotide, deoxyribonucleotide, or nucleotide analog
gaangnnnnaaagcgggcn

SEQ ID NO: 149 - DNA, 19 bp, Artificial Sequence
  based on Homo sapiens. Each nucleobase may be part of a ribonucleotide, deoxyribonucleotide, or nucleotide analog
cacnagagggccagnnaaa

SEQ ID NO: 150 - DNA, 19 bp, Artificial Sequence
  based on Homo sapiens. Each nucleobase may be part of a ribonucleotide, deoxyribonucleotide, or nucleotide analog
ccgcacnngcaagcngcnc

SEQ ID NO: 151 - DNA, 19 bp, Artificial Sequence
  based on Homo sapiens. Each nucleobase may be part of a ribonucleotide, deoxyribonucleotide, or nucleotide analog
cancancacngnnacccac

SEQ ID NO: 152 - DNA, 19 bp, Artificial Sequence
  based on Homo sapiens. Each nucleobase may be part of a ribonucleotide, deoxyribonucleotide, or nucleotide analog
ccaccancacagcaaaagc

SEQ ID NO: 153 - DNA, 19 bp, Artificial Sequence
  based on Homo sapiens. Each nucleobase may be part of a ribonucleotide, deoxyribonucleotide, or nucleotide analog
nccaganncccaacaccng

SEQ ID NO: 154 - DNA, 19 bp, Artificial Sequence
  based on Homo sapiens. Each nucleobase may be part of a ribonucleotide, deoxyribonucleotide, or nucleotide analog
cccanggancancnccaga

SEQ ID NO: 155 - DNA, 19 bp, Artificial Sequence
  based on Homo sapiens. Each nucleobase may be part of a ribonucleotide, deoxyribonucleotide, or nucleotide analog
aaccacnnggcangnngaa

SEQ ID NO: 156 - DNA, 19 bp, Artificial Sequence
  based on Homo sapiens. Each nucleobase may be part of a ribonucleotide, deoxyribonucleotide, or nucleotide analog
caagnacncacaccnngga

SEQ ID NO: 157 - DNA, 19 bp, Artificial Sequence
  based on Homo sapiens. Each nucleobase may be part of a ribonucleotide, deoxyribonucleotide, or nucleotide analog
ccngnccnnnaanncnnan

SEQ ID NO: 158 - DNA, 19 bp, Artificial Sequence
  based on Homo sapiens. Each nucleobase may be part of a ribonucleotide, deoxyribonucleotide, or nucleotide analog
ngaacnngacggangaacn

SEQ ID NO: 159 - DNA, 19 bp, Artificial Sequence
  based on Homo sapiens. Each nucleobase may be part of a ribonucleotide, deoxyribonucleotide, or nucleotide analog
nagangagggnaacnggcn

SEQ ID NO: 160 - DNA, 19 bp, Artificial Sequence
  based on Homo sapiens. Each nucleobase may be part of a ribonucleotide, deoxyribonucleotide, or nucleotide analog
ngganagcagcngnncaag

SEQ ID NO: 161 - DNA, 19 bp, Artificial Sequence
  based on Homo sapiens. Each nucleobase may be part of a ribonucleotide, deoxyribonucleotide, or nucleotide analog
cannnncancnccngggcn

SEQ ID NO: 162 - DNA, 19 bp, Artificial Sequence
  based on Homo sapiens. Each nucleobase may be part of a ribonucleotide, deoxyribonucleotide, or nucleotide analog
ngganaanngangacncng

SEQ ID NO: 163 - DNA, 19 bp, Artificial Sequence
  based on Homo sapiens. Each nucleobase may be part of a ribonucleotide, deoxyribonucleotide, or nucleotide analog
gncnncnccaggnncaaaa

SEQ ID NO: 164 - DNA, 19 bp, Artificial Sequence
  based on Homo sapiens. Each nucleobase may be part of a ribonucleotide, deoxyribonucleotide, or nucleotide analog
nanncancanganngcanc

SEQ ID NO: 165 - DNA, 19 bp, Artificial Sequence
  based on Homo sapiens. Each nucleobase may be part of a ribonucleotide, deoxyribonucleotide, or nucleotide analog
cannnccacggcagcanna

SEQ ID NO: 166 - DNA, 19 bp, Artificial Sequence
  based on Homo sapiens. Each nucleobase may be part of a ribonucleotide, deoxyribonucleotide, or nucleotide analog
ccaggcnncnacnaaagcc

SEQ ID NO: 167 - DNA, 19 bp, Artificial Sequence
  based on Homo sapiens. Each nucleobase may be part of a ribonucleotide, deoxyribonucleotide, or nucleotide analog
gcnaggannnnncncngaa

SEQ ID NO: 168 - DNA, 19 bp, Artificial Sequence
  based on Homo sapiens. Each nucleobase may be part of a ribonucleotide, deoxyribonucleotide, or nucleotide analog
ncnanaanncncnccagnn

SEQ ID NO: 169 - DNA, 19 bp, Artificial Sequence
  based on Homo sapiens. Each nucleobase may be part of a ribonucleotide, deoxyribonucleotide, or nucleotide analog
acacaagancanngacnag

SEQ ID NO: 170 - DNA, 19 bp, Artificial Sequence
  based on Homo sapiens. Each nucleobase may be part of a ribonucleotide, deoxyribonucleotide, or nucleotide analog
ncngcanngagnaagncna

SEQ ID NO: 171 - DNA, 19 bp, Artificial Sequence
  based on Homo sapiens. Each nucleobase may be part of a ribonucleotide, deoxyribonucleotide, or nucleotide analog
cncnncccnnannncancn

SEQ ID NO: 172 - DNA, 19 bp, Artificial Sequence
  based on Homo sapiens. Each nucleobase may be part of a ribonucleotide, deoxyribonucleotide, or nucleotide analog
nccncagnngcncnnncnc

SEQ ID NO: 173 - DNA, 19 bp, Artificial Sequence
  based on Homo sapiens. Each nucleobase may be part of a ribonucleotide, deoxyribonucleotide, or nucleotide analog
gccanncnanncnnccgga

SEQ ID NO: 174 - DNA, 19 bp, Artificial Sequence
  based on Homo sapiens. Each nucleobase may be part of a ribonucleotide, deoxyribonucleotide, or nucleotide analog
agncaaangnngaaaaagn

SEQ ID NO: 175 - DNA, 19 bp, Artificial Sequence
  based on Homo sapiens. Each nucleobase may be part of a ribonucleotide, deoxyribonucleotide, or nucleotide analog
ccaggannggaannacaca

SEQ ID NO: 176 - DNA, 19 bp, Artificial Sequence
  based on Homo sapiens. Each nucleobase may be part of a ribonucleotide, deoxyribonucleotide, or nucleotide analog
annccggcagnnagnagac

SEQ ID NO: 177 - DNA, 19 bp, Artificial Sequence
  based on Homo sapiens. Each nucleobase may be part of a ribonucleotide, deoxyribonucleotide, or nucleotide analog
naacancangnncnngnnc

SEQ ID NO: 178 - DNA, 19 bp, Artificial Sequence
  based on Homo sapiens. Each nucleobase may be part of a ribonucleotide, deoxyribonucleotide, or nucleotide analog
gncngngncnncngnnnaa

SEQ ID NO: 179 - DNA, 19 bp, Artificial Sequence
  based on Homo sapiens. Each nucleobase may be part of a ribonucleotide, deoxyribonucleotide, or nucleotide analog
nncncnngcnngnaaagac

SEQ ID NO: 180 - DNA, 19 bp, Artificial Sequence
  based on Homo sapiens. Each nucleobase may be part of a ribonucleotide, deoxyribonucleotide, or nucleotide analog
cnaaaancgnancaancag

SEQ ID NO: 181 - DNA, 19 bp, Artificial Sequence
  based on Homo sapiens. Each nucleobase may be part of a ribonucleotide, deoxyribonucleotide, or nucleotide analog
ggcngcaanannnccnnnn

SEQ ID NO: 182 - DNA, 19 bp, Artificial Sequence
  based on Homo sapiens. Each nucleobase may be part of a ribonucleotide, deoxyribonucleotide, or nucleotide analog
gagagnnncngaanacagn

SEQ ID NO: 183 - DNA, 19 bp, Artificial Sequence
  based on Homo sapiens. Each nucleobase may be part of a ribonucleotide, deoxyribonucleotide, or nucleotide analog
acagcnncagcnncnngca

SEQ ID NO: 184 - DNA, 19 bp, Artificial Sequence
  based on Homo sapiens. Each nucleobase may be part of a ribonucleotide, deoxyribonucleotide, or nucleotide analog
aaanaaangcncananaac

SEQ ID NO: 185 - DNA, 19 bp, Artificial Sequence
  based on Homo sapiens. Each nucleobase may be part of a ribonucleotide, deoxyribonucleotide, or nucleotide analog
gaaacancnncngngggaa

SEQ ID NO: 186 - DNA, 19 bp, Artificial Sequence
  based on Homo sapiens. Each nucleobase may be part of a ribonucleotide, deoxyribonucleotide, or nucleotide analog
gnncnnccacnggnaganc

SEQ ID NO: 187 - DNA, 19 bp, Artificial Sequence
  based on Homo sapiens. Each nucleobase may be part of a ribonucleotide, deoxyribonucleotide, or nucleotide analog
cnncnngnagncnccgcaa

SEQ ID NO: 188 - DNA, 19 bp, Artificial Sequence
  based on Homo sapiens. Each nucleobase may be part of a ribonucleotide, deoxyribonucleotide, or nucleotide analog
nngnccanacacacnnnac

SEQ ID NO: 189 - DNA, 19 bp, Artificial Sequence
  based on Homo sapiens. Each nucleobase may be part of a ribonucleotide, deoxyribonucleotide, or nucleotide analog
aaccaaannagganaaaag

SEQ ID NO: 190 - DNA, 19 bp, Artificial Sequence
  based on Homo sapiens. Each nucleobase may be part of a ribonucleotide, deoxyribonucleotide, or nucleotide analog
angnncananggnnnagan

SEQ ID NO: 191 - DNA, 19 bp, Artificial Sequence
  based on Homo sapiens. Each nucleobase may be part of a ribonucleotide, deoxyribonucleotide, or nucleotide analog
naagnnnnacnncacnnac

SEQ ID NO: 192 - DNA, 19 bp, Artificial Sequence
  based on Homo sapiens. Each nucleobase may be part of a ribonucleotide, deoxyribonucleotide, or nucleotide analog
angnncccggnannagnac

SEQ ID NO: 193 - DNA, 19 bp, Artificial Sequence
  based on Homo sapiens. Each nucleobase may be part of a ribonucleotide, deoxyribonucleotide, or nucleotide analog
gggcncaagnaanncncnn

SEQ ID NO: 194 - DNA, 19 bp, Artificial Sequence
  based on Homo sapiens. Each nucleobase may be part of a ribonucleotide, deoxyribonucleotide, or nucleotide analog
gcccaggangganncaaac

SEQ ID NO: 195 - DNA, 19 bp, Artificial Sequence
  based on Homo sapiens. Each nucleobase may be part of a ribonucleotide, deoxyribonucleotide, or nucleotide analog
ggnancnccnncaccagna

SEQ ID NO: 196 - DNA, 18 bp, Artificial Sequence
  based on Homo sapiens. Each nucleobase may be part of a ribonucleotide, deoxyribonucleotide, or nucleotide analog
angaccacnnccncangg

SEQ ID NO: 197 - DNA, 15 bp, Artificial Sequence
  based on Homo sapiens. Each nucleobase may be part of a ribonucleotide, deoxyribonucleotide, or nucleotide analog
ganaccagaannngn

SEQ ID NO: 198 - DNA, 15 bp, Artificial Sequence
  based on Homo sapiens. Each nucleobase may be part of a ribonucleotide, deoxyribonucleotide, or nucleotide analog
ngnnnaagaccanag

SEQ ID NO: 199 - DNA, 19 bp, Artificial Sequence
  based on Homo sapiens. Each nucleobase may be part of a ribonucleotide, deoxyribonucleotide, or nucleotide analog
gcngagncnccanacngcc

SEQ ID NO: 200 - DNA, 19 bp, Artificial Sequence
  based on Homo sapiens. Each nucleobase may be part of a ribonucleotide, deoxyribonucleotide, or nucleotide analog
ggcncncngcccacngaan

SEQ ID NO: 201 - DNA, 19 bp, Artificial Sequence
  based on Homo sapiens. Each nucleobase may be part of a ribonucleotide, deoxyribonucleotide, or nucleotide analog
ancnncncnngaaaanagg

SEQ ID NO: 202 - DNA, 19 bp, Artificial Sequence
  based on Homo sapiens. Each nucleobase may be part of a ribonucleotide, deoxyribonucleotide, or nucleotide analog
cagagannncannnaacgn

SEQ ID NO: 203 - DNA, 19 bp, Artificial Sequence
  based on Homo sapiens. Each nucleobase may be part of a ribonucleotide, deoxyribonucleotide, or nucleotide analog
ancnngacnngannanagg

SEQ ID NO: 204 - DNA, 19 bp, Artificial Sequence
  based on Homo sapiens. Each nucleobase may be part of a ribonucleotide, deoxyribonucleotide, or nucleotide analog
gganaaaagnncncnncna

SEQ ID NO: 205 - DNA, 19 bp, Artificial Sequence
  based on Homo sapiens. Each nucleobase may be part of a ribonucleotide, deoxyribonucleotide, or nucleotide analog
cgcacggnancnccnncac

SEQ ID NO: 206 - DNA, 19 bp, Artificial Sequence
  based on Homo sapiens. Each nucleobase may be part of a ribonucleotide, deoxyribonucleotide, or nucleotide analog
cnacgcncgccancgnnca

SEQ ID NO: 207 - DNA, 19 bp, Artificial Sequence
  based on Homo sapiens. Each nucleobase may be part of a ribonucleotide, deoxyribonucleotide, or nucleotide analog
cacnnccncnanggcacgc

SEQ ID NO: 208 - DNA, 19 bp, Artificial Sequence
  based on Homo sapiens. Each nucleobase may be part of a ribonucleotide, deoxyribonucleotide, or nucleotide analog
cgcacccnancnggnncac

SEQ ID NO: 209 - DNA, 19 bp, Artificial Sequence
  based on Homo sapiens. Each nucleobase may be part of a ribonucleotide, deoxyribonucleotide, or nucleotide analog
gcngagncnccananngcc

SEQ ID NO: 210 - DNA, 19 bp, Artificial Sequence
  based on Homo sapiens. Each nucleobase may be part of a ribonucleotide, deoxyribonucleotide, or nucleotide analog
ggcncnnncgccacngaan

SEQ ID NO: 211 - DNA, 19 bp, Artificial Sequence
  based on Homo sapiens. Each nucleobase may be part of a ribonucleotide, deoxyribonucleotide, or nucleotide analog
ccgnnanaccncngagncg

SEQ ID NO: 212 - DNA, 19 bp, Artificial Sequence
  based on Homo sapiens. Each nucleobase may be part of a ribonucleotide, deoxyribonucleotide, or nucleotide analog
gcngacacnccaannngcc

SEQ ID NO: 213 - DNA, 19 bp, Artificial Sequence
  based on Homo sapiens. Each nucleobase may be part of a ribonucleotide, deoxyribonucleotide, or nucleotide analog
accanncnggnaaccagaa

SEQ ID NO: 214 - DNA, 19 bp, Artificial Sequence
  based on Homo sapiens. Each nucleobase may be part of a ribonucleotide, deoxyribonucleotide, or nucleotide analog
ngcccaagaanacnagnca

SEQ ID NO: 215 - DNA, 19 bp, Artificial Sequence
  based on Homo sapiens. Each nucleobase may be part of a ribonucleotide, deoxyribonucleotide, or nucleotide analog
accanagngganngcagaa

SEQ ID NO: 216 - DNA, 19 bp, Artificial Sequence
  based on Homo sapiens. Each nucleobase may be part of a ribonucleotide, deoxyribonucleotide, or nucleotide analog
aagaccanaggncnnacca

SEQ ID NO: 217 - DNA, 22 bp, Artificial Sequence
  based on Homo sapiens. Each nucleobase may be part of a ribonucleotide, deoxyribonucleotide, or nucleotide analog
ganncacnncnncgaanannaa

SEQ ID NO: 218 - DNA, 19 bp, Artificial Sequence
  based on Homo sapiens. Each nucleobase may be part of a ribonucleotide, deoxyribonucleotide, or nucleotide analog
ngaaangnaaancancnnc

SEQ ID NO: 219 - DNA, 19 bp, Artificial Sequence
  based on Homo sapiens. Each nucleobase may be part of a ribonucleotide, deoxyribonucleotide, or nucleotide analog
ganncngnncganaannaa

SEQ ID NO: 220 - DNA, 19 bp, Artificial Sequence
  based on Homo sapiens. Each nucleobase may be part of a ribonucleotide, deoxyribonucleotide, or nucleotide analog
aannanaagcnncacnnag

SEQ ID NO: 221 - DNA, 19 bp, Artificial Sequence
  based on Homo sapiens. Each nucleobase may be part of a ribonucleotide, deoxyribonucleotide, or nucleotide analog
gcngagncnccananngcc

SEQ ID NO: 222 - DNA, 19 bp, Artificial Sequence
  based on Homo sapiens. Each nucleobase may be part of a ribonucleotide, deoxyribonucleotide, or nucleotide analog
ggcncnnngcccacngaan

SEQ ID NO: 223 - DNA, 19 bp, Artificial Sequence
  based on Homo sapiens. Each nucleobase may be part of a ribonucleotide, deoxyribonucleotide, or nucleotide analog
accanncngganaccagaa

SEQ ID NO: 224 - DNA, 19 bp, Artificial Sequence
  based on Homo sapiens. Each nucleobase may be part of a ribonucleotide, deoxyribonucleotide, or nucleotide analog
aagaccanaggncnnacca

SEQ ID NO: 225 - DNA, 19 bp, Artificial Sequence
  based on Homo sapiens. Each nucleobase may be part of a ribonucleotide, deoxyribonucleotide, or nucleotide analog
ancnncncnngaaaanagg

SEQ ID NO: 226 - DNA, 19 bp, Artificial Sequence
  based on Homo sapiens. Each nucleobase may be part of a ribonucleotide, deoxyribonucleotide, or nucleotide analog
gganaaaagnncncnncna

SEQ ID NO: 227 - DNA, 19 bp, Artificial Sequence
  based on Homo sapiens. Each nucleobase may be part of a ribonucleotide, deoxyribonucleotide, or nucleotide analog
ggnancnccnncaccagna

SEQ ID NO: 228 - DNA, 19 bp, Artificial Sequence
  based on Homo sapiens. Each nucleobase may be part of a ribonucleotide, deoxyribonucleotide, or nucleotide analog
angaccacnnccncnangg

SEQ ID NO: 229 - DNA, 19 bp, Artificial Sequence
  based on Homo sapiens. Each nucleobase may be part of a ribonucleotide, deoxyribonucleotide, or nucleotide analog
ncngganaccagaannngn

SEQ ID NO: 230 - DNA, 19 bp, Artificial Sequence
  based on Homo sapiens. Each nucleobase may be part of a ribonucleotide, deoxyribonucleotide, or nucleotide analog
ngnnnaagaccanaggncn

SEQ ID NO: 231 - DNA, 19 bp, Artificial Sequence
  based on Homo sapiens. Each nucleobase may be part of a ribonucleotide, deoxyribonucleotide, or nucleotide analog
gggnnccncgggnanangg

SEQ ID NO: 232 - DNA, 19 bp, Artificial Sequence
  based on Homo sapiens. Each nucleobase may be part of a ribonucleotide, deoxyribonucleotide, or nucleotide analog
ggnananggcgnccnnggg

SEQ ID NO: 233 - DNA, 19 bp, Artificial Sequence
  based on Homo sapiens. Each nucleobase may be part of a ribonucleotide, deoxyribonucleotide, or nucleotide analog
ganncacnncgaanannaa

SEQ ID NO: 234 - DNA, 19 bp, Artificial Sequence
  based on Homo sapiens. Each nucleobase may be part of a ribonucleotide, deoxyribonucleotide, or nucleotide analog
aannanaacgnncacnnag

SEQ ID NO: 235 - DNA, 19 bp, Artificial Sequence
  based on Homo sapiens. Each nucleobase may be part of a ribonucleotide, deoxyribonucleotide, or nucleotide analog
ancnncncnngaaaanagg

SEQ ID NO: 236 - DNA, 19 bp, Artificial Sequence
  based on Homo sapiens. Each nucleobase may be part of a ribonucleotide, deoxyribonucleotide, or nucleotide analog
gcngagncnccananngcc

SEQ ID NO: 237 - DNA, 19 bp, Artificial Sequence
  based on Homo sapiens. Each nucleobase may be part of a ribonucleotide, deoxyribonucleotide, or nucleotide analog
ganncacnncgaanannaa

SEQ ID NO: 238 - DNA, 19 bp, Artificial Sequence
  based on Homo sapiens. Each nucleobase may be part of a ribonucleotide, deoxyribonucleotide, or nucleotide analog
ngcccaagaanacnagnca

SEQ ID NO: 239 - DNA, 19 bp, Artificial Sequence
  based on Homo sapiens. Each nucleobase may be part of a ribonucleotide, deoxyribonucleotide, or nucleotide analog
gcngagncnccananngcc

SEQ ID NO: 240 - DNA, 19 bp, Artificial Sequence
  based on Homo sapiens. Each nucleobase may be part of a ribonucleotide, deoxyribonucleotide, or nucleotide analog
ggcncnnngcccacngaan

SEQ ID NO: 241 - DNA, 19 bp, Artificial Sequence
  based on Homo sapiens. Each nucleobase may be part of a ribonucleotide, deoxyribonucleotide, or nucleotide analog
ggnancnccnncaccagna

SEQ ID NO: 242 - DNA, 19 bp, Artificial Sequence
  based on Homo sapiens. Each nucleobase may be part of a ribonucleotide, deoxyribonucleotide, or nucleotide analog
angaccacnnccncnangg

SEQ ID NO: 243 - DNA, 19 bp, Artificial Sequence
  based on Homo sapiens. Each nucleobase may be part of a ribonucleotide, deoxyribonucleotide, or nucleotide analog
gaaagnaanannnaagcag

SEQ ID NO: 244 - DNA, 19 bp, Artificial Sequence
  based on Homo sapiens. Each nucleobase may be part of a ribonucleotide, deoxyribonucleotide, or nucleotide analog
gagcaannnanaangaaag

SEQ ID NO: 245 - DNA, 19 bp, Artificial Sequence
  based on Homo sapiens. Each nucleobase may be part of a ribonucleotide, deoxyribonucleotide, or nucleotide analog
accgcnaagaaacanncna

SEQ ID NO: 246 - DNA, 19 bp, Artificial Sequence
  based on Homo sapiens. Each nucleobase may be part of a ribonucleotide, deoxyribonucleotide, or nucleotide analog
ancnnacaaagaanccgca

SEQ ID NO: 247 - DNA, 19 bp, Artificial Sequence
  based on Homo sapiens. Each nucleobase may be part of a ribonucleotide, deoxyribonucleotide, or nucleotide analog
nanccacnnangacanaaa

SEQ ID NO: 248 - DNA, 19 bp, Artificial Sequence
  based on Homo sapiens. Each nucleobase may be part of a ribonucleotide, deoxyribonucleotide, or nucleotide analog
aaanacagnanncaccnan

SEQ ID NO: 249 - DNA, 19 bp, Artificial Sequence
  based on Homo sapiens. Each nucleobase may be part of a ribonucleotide, deoxyribonucleotide, or nucleotide analog
ngcacccngganaccannn

SEQ ID NO: 250 - DNA, 19 bp, Artificial Sequence
  based on Homo sapiens. Each nucleobase may be part of a ribonucleotide, deoxyribonucleotide, or nucleotide analog
nnnaccanaggncccagcn

SEQ ID NO: 251 - DNA, 19 bp, Artificial Sequence
  based on Homo sapiens. Each nucleobase may be part of a ribonucleotide, deoxyribonucleotide, or nucleotide analog
gcngagncnccanacngcc

SEQ ID NO: 252 - DNA, 19 bp, Artificial Sequence
  based on Homo sapiens. Each nucleobase may be part of a ribonucleotide, deoxyribonucleotide, or nucleotide analog
ggcncncngcccacngaan

SEQ ID NO: 253 - DNA, 19 bp, Artificial Sequence
  based on Homo sapiens. Each nucleobase may be part of a ribonucleotide, deoxyribonucleotide, or nucleotide analog
annggnnccaangngnncn

SEQ ID NO: 254 - DNA, 19 bp, Artificial Sequence
  based on Homo sapiens. Each nucleobase may be part of a ribonucleotide, deoxyribonucleotide, or nucleotide analog
ncnngngnaaccnnggnna

SEQ ID NO: 255 - DNA, 19 bp, Artificial Sequence
  based on Homo sapiens. Each nucleobase may be part of a ribonucleotide, deoxyribonucleotide, or nucleotide analog
acaggacnaccacnnggaa

SEQ ID NO: 256 - DNA, 19 bp, Artificial Sequence
  based on Homo sapiens. Each nucleobase may be part of a ribonucleotide, deoxyribonucleotide, or nucleotide analog
aaggnncaccancaggaca

SEQ ID NO: 257 - DNA, 19 bp, Artificial Sequence
  based on Homo sapiens. Each nucleobase may be part of a ribonucleotide, deoxyribonucleotide, or nucleotide analog
aagcacngcacnnggncac

SEQ ID NO: 258 - DNA, 19 bp, Artificial Sequence
  based on Homo sapiens. Each nucleobase may be part of a ribonucleotide, deoxyribonucleotide, or nucleotide analog
cacnggnngaccncacaag

SEQ ID NO: 259 - DNA, 19 bp, Artificial Sequence
  based on Homo sapiens. Each nucleobase may be part of a ribonucleotide, deoxyribonucleotide, or nucleotide analog
ngncagnacangnnggcnc

SEQ ID NO: 260 - DNA, 19 bp, Artificial Sequence
  based on Homo sapiens. Each nucleobase may be part of a ribonucleotide, deoxyribonucleotide, or nucleotide analog
cnaggnngnccangacngn

SEQ ID NO: 261 - DNA, 19 bp, Artificial Sequence
  based on Homo sapiens. Each nucleobase may be part of a ribonucleotide, deoxyribonucleotide, or nucleotide analog
ancnncncnngaaaanagg

SEQ ID NO: 262 - DNA, 19 bp, Artificial Sequence
  based on Homo sapiens. Each nucleobase may be part of a ribonucleotide, deoxyribonucleotide, or nucleotide analog
ancnncncnngaaaanagg

SEQ ID NO: 263 - DNA, 19 bp, Artificial Sequence
  based on Homo sapiens. Each nucleobase may be part of a ribonucleotide, deoxyribonucleotide, or nucleotide analog
gganaaaagnncncnncna

SEQ ID NO: 264 - DNA, 19 bp, Artificial Sequence
  based on Homo sapiens. Each nucleobase may be part of a ribonucleotide, deoxyribonucleotide, or nucleotide analog
gcngagncnccananngcc

SEQ ID NO: 265 - DNA, 19 bp, Artificial Sequence
  based on Homo sapiens. Each nucleobase may be part of a ribonucleotide, deoxyribonucleotide, or nucleotide analog
gcngagncnccananngcc

SEQ ID NO: 266 - DNA, 19 bp, Artificial Sequence
  based on Homo sapiens. Each nucleobase may be part of a ribonucleotide, deoxyribonucleotide, or nucleotide analog
ggcncnnngcccacngaan

SEQ ID NO: 267 - DNA, 19 bp, Artificial Sequence
  based on Homo sapiens. Each nucleobase may be part of a ribonucleotide, deoxyribonucleotide, or nucleotide analog
accanncngganaccagaa

SEQ ID NO: 268 - DNA, 19 bp, Artificial Sequence
  based on Homo sapiens. Each nucleobase may be part of a ribonucleotide, deoxyribonucleotide, or nucleotide analog
accanncngganaccagaa

SEQ ID NO: 269 - DNA, 19 bp, Artificial Sequence
  based on Homo sapiens. Each nucleobase may be part of a ribonucleotide, deoxyribonucleotide, or nucleotide analog
aagaccanaggncnnacca

SEQ ID NO: 270 - DNA, 19 bp, Artificial Sequence
  based on Homo sapiens. Each nucleobase may be part of a ribonucleotide, deoxyribonucleotide, or nucleotide analog
gggnnccncgggnanangg

SEQ ID NO: 271 - DNA, 19 bp, Artificial Sequence
  based on Homo sapiens. Each nucleobase may be part of a ribonucleotide, deoxyribonucleotide, or nucleotide analog
ggnananggcgnccnnggg

SEQ ID NO: 272 - DNA, 19 bp, Artificial Sequence
  based on Homo sapiens. Each nucleobase may be part of a ribonucleotide, deoxyribonucleotide, or nucleotide analog
ggnancnccnncaccagna

SEQ ID NO: 273 - DNA, 19 bp, Artificial Sequence
  based on Homo sapiens. Each nucleobase may be part of a ribonucleotide, deoxyribonucleotide, or nucleotide analog
angaccacnnccncnangg

SEQ ID NO: 274 - DNA, 19 bp, Artificial Sequence
  based on Homo sapiens. Each nucleobase may be part of a ribonucleotide, deoxyribonucleotide, or nucleotide analog
ganncacnncgaanannaa

SEQ ID NO: 275 - DNA, 19 bp, Artificial Sequence
  based on Homo sapiens. Each nucleobase may be part of a ribonucleotide, deoxyribonucleotide, or nucleotide analog
aannanaacgnncacnnag

SEQ ID NO: 276 - DNA, 19 bp, Artificial Sequence
  based on Homo sapiens. Each nucleobase may be part of a ribonucleotide, deoxyribonucleotide, or nucleotide analog
agcaaggacaagcccagnc

SEQ ID NO: 277 - DNA, 19 bp, Artificial Sequence
  based on Homo sapiens. Each nucleobase may be part of a ribonucleotide, deoxyribonucleotide, or nucleotide analog
ngnaaaccngcngcccaga

SEQ ID NO: 278 - DNA, 19 bp, Artificial Sequence
  based on Homo sapiens. Each nucleobase may be part of a ribonucleotide, deoxyribonucleotide, or nucleotide analog
agaagncgnnnnccnccnn

SEQ ID NO: 279 - DNA, 19 bp, Artificial Sequence
  based on Homo sapiens. Each nucleobase may be part of a ribonucleotide, deoxyribonucleotide, or nucleotide analog
ccgagannagacnaagncc

SEQ ID NO: 280 - DNA, 19 bp, Artificial Sequence
  based on Homo sapiens. Each nucleobase may be part of a ribonucleotide, deoxyribonucleotide, or nucleotide analog
acnnnnccnnnannnccac

SEQ ID NO: 281 - DNA, 19 bp, Artificial Sequence
  based on Homo sapiens. Each nucleobase may be part of a ribonucleotide, deoxyribonucleotide, or nucleotide analog
ncccaaacacaggnacnan

SEQ ID NO: 282 - DNA, 19 bp, Artificial Sequence
  based on Homo sapiens. Each nucleobase may be part of a ribonucleotide, deoxyribonucleotide, or nucleotide analog
canncncagcggnaacagc

SEQ ID NO: 283 - DNA, 19 bp, Artificial Sequence
  based on Homo sapiens. Each nucleobase may be part of a ribonucleotide, deoxyribonucleotide, or nucleotide analog
accancanncncanccnca

SEQ ID NO: 284 - DNA, 19 bp, Artificial Sequence
  based on Homo sapiens. Each nucleobase may be part of a ribonucleotide, deoxyribonucleotide, or nucleotide analog
aangnaaccnncaaccanc

SEQ ID NO: 285 - DNA, 18 bp, Artificial Sequence
  based on Homo sapiens. Each nucleobase may be part of a ribonucleotide, deoxyribonucleotide, or nucleotide analog
nnngnanncancacngnc

SEQ ID NO: 286 - DNA, 18 bp, Artificial Sequence
  based on Homo sapiens. Each nucleobase may be part of a ribonucleotide, deoxyribonucleotide, or nucleotide analog
ncacancncannaccaac

SEQ ID NO: 287 - DNA, 19 bp, Artificial Sequence
  based on Homo sapiens. Each nucleobase may be part of a ribonucleotide, deoxyribonucleotide, or nucleotide analog
ccaggnggcaggagaaaca

SEQ ID NO: 288 - DNA, 19 bp, Artificial Sequence
  based on Homo sapiens. Each nucleobase may be part of a ribonucleotide, deoxyribonucleotide, or nucleotide analog
ngcagacnncaangcnnng

SEQ ID NO: 289 - DNA, 19 bp, Artificial Sequence
  based on Homo sapiens. Each nucleobase may be part of a ribonucleotide, deoxyribonucleotide, or nucleotide analog
naagcaagncacngnggcn

SEQ ID NO: 290 - DNA, 19 bp, Artificial Sequence
  based on Homo sapiens. Each nucleobase may be part of a ribonucleotide, deoxyribonucleotide, or nucleotide analog
cngagncganaanacnagc

SEQ ID NO: 291 - DNA, 19 bp, Artificial Sequence
  based on Homo sapiens. Each nucleobase may be part of a ribonucleotide, deoxyribonucleotide, or nucleotide analog
acnagccannagnaaagag

SEQ ID NO: 292 - DNA, 19 bp, Artificial Sequence
  based on Homo sapiens. Each nucleobase may be part of a ribonucleotide, deoxyribonucleotide, or nucleotide analog
caacagcagagaccnngnc

SEQ ID NO: 293 - DNA, 19 bp, Artificial Sequence
  based on Homo sapiens. Each nucleobase may be part of a ribonucleotide, deoxyribonucleotide, or nucleotide analog
anagcanaccnngaaccag

SEQ ID NO: 294 - DNA, 19 bp, Artificial Sequence
  based on Homo sapiens. Each nucleobase may be part of a ribonucleotide, deoxyribonucleotide, or nucleotide analog
cancngnaggcnaagangg

SEQ ID NO: 295 - DNA, 19 bp, Artificial Sequence
  based on Homo sapiens. Each nucleobase may be part of a ribonucleotide, deoxyribonucleotide, or nucleotide analog
agnnaccagangccancng

SEQ ID NO: 296 - DNA, 19 bp, Artificial Sequence
  based on Homo sapiens. Each nucleobase may be part of a ribonucleotide, deoxyribonucleotide, or nucleotide analog
aancnacncnganagngga

SEQ ID NO: 297 - DNA, 19 bp, Artificial Sequence
  based on Homo sapiens. Each nucleobase may be part of a ribonucleotide, deoxyribonucleotide, or nucleotide analog
gnnncngaagccaacanca

SEQ ID NO: 298 - DNA, 19 bp, Artificial Sequence
  based on Homo sapiens. Each nucleobase may be part of a ribonucleotide, deoxyribonucleotide, or nucleotide analog
ncaacnnancaccnccnga

SEQ ID NO: 299 - DNA, 19 bp, Artificial Sequence
  based on Homo sapiens. Each nucleobase may be part of a ribonucleotide, deoxyribonucleotide, or nucleotide analog
aagaacnaacanngnagag

SEQ ID NO: 300 - DNA, 19 bp, Artificial Sequence
  based on Homo sapiens. Each nucleobase may be part of a ribonucleotide, deoxyribonucleotide, or nucleotide analog
gnagacaacaggngcngca

SEQ ID NO: 301 - DNA, 19 bp, Artificial Sequence
  based on Homo sapiens. Each nucleobase may be part of a ribonucleotide, deoxyribonucleotide, or nucleotide analog
angnccncngnaannangg

SEQ ID NO: 302 - DNA, 19 bp, Artificial Sequence
  based on Homo sapiens. Each nucleobase may be part of a ribonucleotide, deoxyribonucleotide, or nucleotide analog
nacnnggcnagaacangga

SEQ ID NO: 303 - DNA, 19 bp, Artificial Sequence
  based on Homo sapiens. Each nucleobase may be part of a ribonucleotide, deoxyribonucleotide, or nucleotide analog
gaagcaacncaangnnaag

SEQ ID NO: 304 - DNA, 19 bp, Artificial Sequence
  based on Homo sapiens. Each nucleobase may be part of a ribonucleotide, deoxyribonucleotide, or nucleotide analog
nnnggncnnnnggacncag

SEQ ID NO: 305 - DNA, 19 bp, Artificial Sequence
  based on Homo sapiens. Each nucleobase may be part of a ribonucleotide, deoxyribonucleotide, or nucleotide analog
ccanagancancaggaana

SEQ ID NO: 306 - DNA, 19 bp, Artificial Sequence
  based on Homo sapiens. Each nucleobase may be part of a ribonucleotide, deoxyribonucleotide, or nucleotide analog
caggacnggcnaacacanc

SEQ ID NO: 307 - DNA, 19 bp, Artificial Sequence
  based on Homo sapiens. Each nucleobase may be part of a ribonucleotide, deoxyribonucleotide, or nucleotide analog
nnnaanggcaggcancncc

SEQ ID NO: 308 - DNA, 19 bp, Artificial Sequence
  based on Homo sapiens. Each nucleobase may be part of a ribonucleotide, deoxyribonucleotide, or nucleotide analog
nnaagccancaggangcca

SEQ ID NO: 309 - DNA, 19 bp, Artificial Sequence
  based on Homo sapiens. Each nucleobase may be part of a ribonucleotide, deoxyribonucleotide, or nucleotide analog
gcnacagagnaagcngngn

SEQ ID NO: 310 - DNA, 19 bp, Artificial Sequence
  based on Homo sapiens. Each nucleobase may be part of a ribonucleotide, deoxyribonucleotide, or nucleotide analog
cncnagggaggnagnnnng

SEQ ID NO: 311 - DNA, 19 bp, Artificial Sequence
  based on Homo sapiens. Each nucleobase may be part of a ribonucleotide, deoxyribonucleotide, or nucleotide analog
aagaaaagggacnagccnn

SEQ ID NO: 312 - DNA, 19 bp, Artificial Sequence
  based on Homo sapiens. Each nucleobase may be part of a ribonucleotide, deoxyribonucleotide, or nucleotide analog
cagnncacangacaagncg

SEQ ID NO: 313 - DNA, 19 bp, Artificial Sequence
  based on Homo sapiens. Each nucleobase may be part of a ribonucleotide, deoxyribonucleotide, or nucleotide analog
gacnccnnncngagacagg

SEQ ID NO: 314 - DNA, 19 bp, Artificial Sequence
  based on Homo sapiens. Each nucleobase may be part of a ribonucleotide, deoxyribonucleotide, or nucleotide analog
anncacaccagngnaanag

SEQ ID NO: 315 - DNA, 19 bp, Artificial Sequence
  based on Homo sapiens. Each nucleobase may be part of a ribonucleotide, deoxyribonucleotide, or nucleotide analog
cagaagcannngaccnngn

SEQ ID NO: 316 - DNA, 19 bp, Artificial Sequence
  based on Homo sapiens. Each nucleobase may be part of a ribonucleotide, deoxyribonucleotide, or nucleotide analog
ccagcancaggccacaaca

SEQ ID NO: 317 - DNA, 19 bp, Artificial Sequence
  based on Homo sapiens. Each nucleobase may be part of a ribonucleotide, deoxyribonucleotide, or nucleotide analog
nnncagnaggacngncncc

SEQ ID NO: 318 - DNA, 19 bp, Artificial Sequence
  based on Homo sapiens. Each nucleobase may be part of a ribonucleotide, deoxyribonucleotide, or nucleotide analog
ngcagcnagganacaacnn

SEQ ID NO: 319 - DNA, 19 bp, Artificial Sequence
  based on Homo sapiens. Each nucleobase may be part of a ribonucleotide, deoxyribonucleotide, or nucleotide analog
agaggnagcnnccaagnng

SEQ ID NO: 320 - DNA, 19 bp, Artificial Sequence
  based on Homo sapiens. Each nucleobase may be part of a ribonucleotide, deoxyribonucleotide, or nucleotide analog
gaagnaangagngngngga

SEQ ID NO: 321 - DNA, 19 bp, Artificial Sequence
  based on Homo sapiens. Each nucleobase may be part of a ribonucleotide, deoxyribonucleotide, or nucleotide analog
ggannnganggagagnnng

SEQ ID NO: 322 - DNA, 19 bp, Artificial Sequence
  based on Homo sapiens. Each nucleobase may be part of a ribonucleotide, deoxyribonucleotide, or nucleotide analog
gaacnncncancaaggcag

SEQ ID NO: 323 - DNA, 19 bp, Artificial Sequence
  based on Homo sapiens. Each nucleobase may be part of a ribonucleotide, deoxyribonucleotide, or nucleotide analog
aggnccnangnagnaaaag

SEQ ID NO: 324 - DNA, 19 bp, Artificial Sequence
  based on Homo sapiens. Each nucleobase may be part of a ribonucleotide, deoxyribonucleotide, or nucleotide analog
caannnnccaccacaggca

SEQ ID NO: 325 - DNA, 19 bp, Artificial Sequence
  based on Homo sapiens. Each nucleobase may be part of a ribonucleotide, deoxyribonucleotide, or nucleotide analog
cannanccnncggnnccca

SEQ ID NO: 326 - DNA, 19 bp, Artificial Sequence
  based on Homo sapiens. Each nucleobase may be part of a ribonucleotide, deoxyribonucleotide, or nucleotide analog
cncaggngnncngacanag

SEQ ID NO: 327 - DNA, 19 bp, Artificial Sequence
  based on Homo sapiens. Each nucleobase may be part of a ribonucleotide, deoxyribonucleotide, or nucleotide analog
gcncagannagaaacngng

SEQ ID NO: 328 - DNA, 19 bp, Artificial Sequence
  based on Homo sapiens. Each nucleobase may be part of a ribonucleotide, deoxyribonucleotide, or nucleotide analog
cngcangngncngcangcn

SEQ ID NO: 329 - DNA, 19 bp, Artificial Sequence
  based on Homo sapiens. Each nucleobase may be part of a ribonucleotide, deoxyribonucleotide, or nucleotide analog
nnaacnagaacacnagagg

SEQ ID NO: 330 - DNA, 19 bp, Artificial Sequence
  based on Homo sapiens. Each nucleobase may be part of a ribonucleotide, deoxyribonucleotide, or nucleotide analog
canaanaaaaacccgcacn

SEQ ID NO: 331 - DNA, 19 bp, Artificial Sequence
  based on Homo sapiens. Each nucleobase may be part of a ribonucleotide, deoxyribonucleotide, or nucleotide analog
caccancacagcaaaagca

SEQ ID NO: 332 - DNA, 19 bp, Artificial Sequence
  based on Homo sapiens. Each nucleobase may be part of a ribonucleotide, deoxyribonucleotide, or nucleotide analog
cnccaganncccaacaccn

SEQ ID NO: 333 - DNA, 19 bp, Artificial Sequence
  based on Homo sapiens. Each nucleobase may be part of a ribonucleotide, deoxyribonucleotide, or nucleotide analog
ggaaaccacnnggcangnn

SEQ ID NO: 334 - DNA, 19 bp, Artificial Sequence
  based on Homo sapiens. Each nucleobase may be part of a ribonucleotide, deoxyribonucleotide, or nucleotide analog
gnncaagnagangagggna

SEQ ID NO: 335 - DNA, 19 bp, Artificial Sequence
  based on Homo sapiens. Each nucleobase may be part of a ribonucleotide, deoxyribonucleotide, or nucleotide analog
ganaanngangacncngca

SEQ ID NO: 336 - DNA, 19 bp, Artificial Sequence
  based on Homo sapiens. Each nucleobase may be part of a ribonucleotide, deoxyribonucleotide, or nucleotide analog
anggncnncnccaggnnca

SEQ ID NO: 337 - DNA, 19 bp, Artificial Sequence
  based on Homo sapiens. Each nucleobase may be part of a ribonucleotide, deoxyribonucleotide, or nucleotide analog
gcannaancacaggggnan

SEQ ID NO: 338 - DNA, 19 bp, Artificial Sequence
  based on Homo sapiens. Each nucleobase may be part of a ribonucleotide, deoxyribonucleotide, or nucleotide analog
naaagcccannnccacggc

SEQ ID NO: 339 - DNA, 19 bp, Artificial Sequence
  based on Homo sapiens. Each nucleobase may be part of a ribonucleotide, deoxyribonucleotide, or nucleotide analog
nnaagccancaggangcca

SEQ ID NO: 340 - DNA, 19 bp, Artificial Sequence
  based on Homo sapiens. Each nucleobase may be part of a ribonucleotide, deoxyribonucleotide, or nucleotide analog
gannnnncncngaacngnc

SEQ ID NO: 341 - DNA, 19 bp, Artificial Sequence
  based on Homo sapiens. Each nucleobase may be part of a ribonucleotide, deoxyribonucleotide, or nucleotide analog
cnanaanncncnccagnng

SEQ ID NO: 342 - DNA, 19 bp, Artificial Sequence
  based on Homo sapiens. Each nucleobase may be part of a ribonucleotide, deoxyribonucleotide, or nucleotide analog
acacaagancanngacnag

SEQ ID NO: 343 - DNA, 19 bp, Artificial Sequence
  based on Homo sapiens. Each nucleobase may be part of a ribonucleotide, deoxyribonucleotide, or nucleotide analog
ncngcanngagnaagncna

SEQ ID NO: 344 - DNA, 19 bp, Artificial Sequence
  based on Homo sapiens. Each nucleobase may be part of a ribonucleotide, deoxyribonucleotide, or nucleotide analog
ncnnnnnccncagnngcnc

SEQ ID NO: 345 - DNA, 19 bp, Artificial Sequence
  based on Homo sapiens. Each nucleobase may be part of a ribonucleotide, deoxyribonucleotide, or nucleotide analog
gngccanncnanncnnccg

SEQ ID NO: 346 - DNA, 19 bp, Artificial Sequence
  based on Homo sapiens. Each nucleobase may be part of a ribonucleotide, deoxyribonucleotide, or nucleotide analog
gnagacnanccagganngg

SEQ ID NO: 347 - DNA, 19 bp, Artificial Sequence
  based on Homo sapiens. Each nucleobase may be part of a ribonucleotide, deoxyribonucleotide, or nucleotide analog
agnncncnngcnngnaaag

SEQ ID NO: 348 - DNA, 19 bp, Artificial Sequence
  based on Homo sapiens. Each nucleobase may be part of a ribonucleotide, deoxyribonucleotide, or nucleotide analog
ncgnancaancagnncncn

SEQ ID NO: 349 - DNA, 19 bp, Artificial Sequence
  based on Homo sapiens. Each nucleobase may be part of a ribonucleotide, deoxyribonucleotide, or nucleotide analog
gcagagagnnncngaanac

SEQ ID NO: 350 - DNA, 19 bp, Artificial Sequence
  based on Homo sapiens. Each nucleobase may be part of a ribonucleotide, deoxyribonucleotide, or nucleotide analog
angnccngnngcacaaana

SEQ ID NO: 351 - DNA, 19 bp, Artificial Sequence
  based on Homo sapiens. Each nucleobase may be part of a ribonucleotide, deoxyribonucleotide, or nucleotide analog
cngaaacancnncngnggg

SEQ ID NO: 352 - DNA, 19 bp, Artificial Sequence
  based on Homo sapiens. Each nucleobase may be part of a ribonucleotide, deoxyribonucleotide, or nucleotide analog
nnncnncnngnagncnccg

SEQ ID NO: 353 - DNA, 19 bp, Artificial Sequence
  based on Homo sapiens. Each nucleobase may be part of a ribonucleotide, deoxyribonucleotide, or nucleotide analog
cnncnnngnccanacacac

SEQ ID NO: 354 - DNA, 19 bp, Artificial Sequence
  based on Homo sapiens. Each nucleobase may be part of a ribonucleotide, deoxyribonucleotide, or nucleotide analog
ggaanaaacacnanggaca

SEQ ID NO: 355 - DNA, 19 bp, Artificial Sequence
  based on Homo sapiens. Each nucleobase may be part of a ribonucleotide, deoxyribonucleotide, or nucleotide analog
canacnacnagangaccac

SEQ ID NO: 356 - DNA, 19 bp, Artificial Sequence
  based on Homo sapiens. Each nucleobase may be part of a ribonucleotide, deoxyribonucleotide, or nucleotide analog
ngnacccnnganngnacnc

SEQ ID NO: 357 - DNA, 19 bp, Artificial Sequence
  based on Homo sapiens. Each nucleobase may be part of a ribonucleotide, deoxyribonucleotide, or nucleotide analog
gaaangnacgaacngnacc

SEQ ID NO: 358 - DNA, 19 bp, Artificial Sequence
  based on Homo sapiens. Each nucleobase may be part of a ribonucleotide, deoxyribonucleotide, or nucleotide analog
gangnnnnggnncnncnnc

SEQ ID NO: 359 - DNA, 19 bp, Artificial Sequence
  based on Homo sapiens. Each nucleobase may be part of a ribonucleotide, deoxyribonucleotide, or nucleotide analog
cnancanncncnnagnnnc

SEQ ID NO: 360 - DNA, 19 bp, Artificial Sequence
  based on Homo sapiens. Each nucleobase may be part of a ribonucleotide, deoxyribonucleotide, or nucleotide analog
acaccnggcnncangnncc

SEQ ID NO: 361 - DNA, 19 bp, Artificial Sequence
  based on Homo sapiens. Each nucleobase may be part of a ribonucleotide, deoxyribonucleotide, or nucleotide analog
gacnacaggcacanaccac

SEQ ID NO: 362 - DNA, 19 bp, Artificial Sequence
  based on Homo sapiens. Each nucleobase may be part of a ribonucleotide, deoxyribonucleotide, or nucleotide analog
ngccncagccngggacnac

SEQ ID NO: 363 - DNA, 19 bp, Artificial Sequence
  based on Homo sapiens. Each nucleobase may be part of a ribonucleotide, deoxyribonucleotide, or nucleotide analog
aggangganncaaacnccn

SEQ ID NO: 364 - DNA, 19 bp, Artificial Sequence
  based on Homo sapiens. Each nucleobase may be part of a ribonucleotide, deoxyribonucleotide, or nucleotide analog
gagaaangngncccnggng

SEQ ID NO: 365 - DNA, 19 bp, Artificial Sequence
  based on Homo sapiens. Each nucleobase may be part of a ribonucleotide, deoxyribonucleotide, or nucleotide analog
gccacaacagaagcannng

SEQ ID NO: 366 - DNA, 19 bp, Artificial Sequence
  based on Homo sapiens. Each nucleobase may be part of a ribonucleotide, deoxyribonucleotide, or nucleotide analog
nncngaaaacncnncaang

SEQ ID NO: 367 - DNA, 19 bp, Artificial Sequence
  based on Homo sapiens. Each nucleobase may be part of a ribonucleotide, deoxyribonucleotide, or nucleotide analog
cnnagcanaaagnancagn

SEQ ID NO: 368 - DNA, 19 bp, Artificial Sequence
  based on Homo sapiens. Each nucleobase may be part of a ribonucleotide, deoxyribonucleotide, or nucleotide analog
caaaaaagnacngcnnagc

SEQ ID NO: 369 - DNA, 19 bp, Artificial Sequence
  based on Homo sapiens. Each nucleobase may be part of a ribonucleotide, deoxyribonucleotide, or nucleotide analog
caaganaaaacnngnccnn

SEQ ID NO: 370 - DNA, 19 bp, Artificial Sequence
  based on Homo sapiens. Each nucleobase may be part of a ribonucleotide, deoxyribonucleotide, or nucleotide analog
nancagncangnngnaaac

SEQ ID NO: 371 - DNA, 19 bp, Artificial Sequence
  based on Homo sapiens. Each nucleobase may be part of a ribonucleotide, deoxyribonucleotide, or nucleotide analog
cnaaanaaccngnncanca

SEQ ID NO: 372 - DNA, 19 bp, Artificial Sequence
  based on Homo sapiens. Each nucleobase may be part of a ribonucleotide, deoxyribonucleotide, or nucleotide analog
agcacacnnnnnacacngc

SEQ ID NO: 373 - DNA, 19 bp, Artificial Sequence
  based on Homo sapiens. Each nucleobase may be part of a ribonucleotide, deoxyribonucleotide, or nucleotide analog
accacnannanncnnganc

SEQ ID NO: 374 - DNA, 19 bp, Artificial Sequence
  based on Homo sapiens. Each nucleobase may be part of a ribonucleotide, deoxyribonucleotide, or nucleotide analog
ngnannngnnnccannncc

SEQ ID NO: 375 - DNA, 19 bp, Artificial Sequence
  based on Homo sapiens. Each nucleobase may be part of a ribonucleotide, deoxyribonucleotide, or nucleotide analog
acngnaaacncnancnnng

SEQ ID NO: 376 - DNA, 19 bp, Artificial Sequence
  based on Homo sapiens. Each nucleobase may be part of a ribonucleotide, deoxyribonucleotide, or nucleotide analog
cnnaagngggcnaaannac

SEQ ID NO: 377 - DNA, 19 bp, Artificial Sequence
  based on Homo sapiens. Each nucleobase may be part of a ribonucleotide, deoxyribonucleotide, or nucleotide analog
ccnncananggncacacna

SEQ ID NO: 378 - DNA, 19 bp, Artificial Sequence
  based on Homo sapiens. Each nucleobase may be part of a ribonucleotide, deoxyribonucleotide, or nucleotide analog
ggnnacaagcnangaagcc

SEQ ID NO: 379 - DNA, 19 bp, Artificial Sequence
  based on Homo sapiens. Each nucleobase may be part of a ribonucleotide, deoxyribonucleotide, or nucleotide analog
cnaagcaacnanagaanac

SEQ ID NO: 380 - DNA, 19 bp, Artificial Sequence
  based on Homo sapiens. Each nucleobase may be part of a ribonucleotide, deoxyribonucleotide, or nucleotide analog
nccnngannnnncacagag

SEQ ID NO: 381 - DNA, 19 bp, Artificial Sequence
  based on Homo sapiens. Each nucleobase may be part of a ribonucleotide, deoxyribonucleotide, or nucleotide analog
anacnaacnnaaagcccng

SEQ ID NO: 382 - DNA, 19 bp, Artificial Sequence
  based on Homo sapiens. Each nucleobase may be part of a ribonucleotide, deoxyribonucleotide, or nucleotide analog
gggnngnagnaacncnnnc

SEQ ID NO: 383 - DNA, 19 bp, Artificial Sequence
  based on Homo sapiens. Each nucleobase may be part of a ribonucleotide, deoxyribonucleotide, or nucleotide analog
nagaacacaacncnnnggg

SEQ ID NO: 384 - DNA, 19 bp, Artificial Sequence
  based on Homo sapiens. Each nucleobase may be part of a ribonucleotide, deoxyribonucleotide, or nucleotide analog
cncngaannnccaaganac

SEQ ID NO: 385 - DNA, 19 bp, Artificial Sequence
  based on Homo sapiens. Each nucleobase may be part of a ribonucleotide, deoxyribonucleotide, or nucleotide analog
nnnacnggannnancncag

SEQ ID NO: 386 - DNA, 19 bp, Artificial Sequence
  based on Homo sapiens. Each nucleobase may be part of a ribonucleotide, deoxyribonucleotide, or nucleotide analog
ngagnaggngacagngcng

SEQ ID NO: 387 - DNA, 19 bp, Artificial Sequence
  based on Homo sapiens. Each nucleobase may be part of a ribonucleotide, deoxyribonucleotide, or nucleotide analog
ggaggcagnnnngngcang

SEQ ID NO: 388 - DNA, 19 bp, Artificial Sequence
  based on Homo sapiens. Each nucleobase may be part of a ribonucleotide, deoxyribonucleotide, or nucleotide analog
cnancnnccannanacncn

SEQ ID NO: 389 - DNA, 19 bp, Artificial Sequence
  based on Homo sapiens. Each nucleobase may be part of a ribonucleotide, deoxyribonucleotide, or nucleotide analog
nngnnngnngcngnnngnc

SEQ ID NO: 390 - DNA, 19 bp, Artificial Sequence
  based on Homo sapiens. Each nucleobase may be part of a ribonucleotide, deoxyribonucleotide, or nucleotide analog
nccnnncngagacaggcac

SEQ ID NO: 391 - DNA, 19 bp, Artificial Sequence
  based on Homo sapiens. Each nucleobase may be part of a ribonucleotide, deoxyribonucleotide, or nucleotide analog
accagcacgagcaagacnc

SEQ ID NO: 392 - DNA, 19 bp, Artificial Sequence
  based on Homo sapiens. Each nucleobase may be part of a ribonucleotide, deoxyribonucleotide, or nucleotide analog
accnngncanncacaccag

SEQ ID NO: 393 - DNA, 19 bp, Artificial Sequence
  based on Homo sapiens. Each nucleobase may be part of a ribonucleotide, deoxyribonucleotide, or nucleotide analog
nccagnnanccagcancag

SEQ ID NO: 394 - DNA, 19 bp, Artificial Sequence
  based on Homo sapiens. Each nucleobase may be part of a ribonucleotide, deoxyribonucleotide, or nucleotide analog
gcnnnngaanaggacngnc

SEQ ID NO: 395 - DNA, 19 bp, Artificial Sequence
  based on Homo sapiens. Each nucleobase may be part of a ribonucleotide, deoxyribonucleotide, or nucleotide analog
gagangncnncaacngcnc

SEQ ID NO: 396 - DNA, 19 bp, Artificial Sequence
  based on Homo sapiens. Each nucleobase may be part of a ribonucleotide, deoxyribonucleotide, or nucleotide analog
ggggnnagnccncgangaa

SEQ ID NO: 397 - DNA, 19 bp, Artificial Sequence
  based on Homo sapiens. Each nucleobase may be part of a ribonucleotide, deoxyribonucleotide, or nucleotide analog
ncanngcanaacngnaggg

SEQ ID NO: 398 - DNA, 19 bp, Artificial Sequence
  based on Homo sapiens. Each nucleobase may be part of a ribonucleotide, deoxyribonucleotide, or nucleotide analog
gcncnngccaanncngang

SEQ ID NO: 399 - DNA, 19 bp, Artificial Sequence
  based on Homo sapiens. Each nucleobase may be part of a ribonucleotide, deoxyribonucleotide, or nucleotide analog
acccnancnccaggnccna

SEQ ID NO: 400 - DNA, 19 bp, Artificial Sequence
  based on Homo sapiens. Each nucleobase may be part of a ribonucleotide, deoxyribonucleotide, or nucleotide analog
acaggcaaagcaggcnacc

SEQ ID NO: 401 - DNA, 19 bp, Artificial Sequence
  based on Homo sapiens. Each nucleobase may be part of a ribonucleotide, deoxyribonucleotide, or nucleotide analog
gnncngacanagcancanc

SEQ ID NO: 402 - DNA, 19 bp, Artificial Sequence
  based on Homo sapiens. Each nucleobase may be part of a ribonucleotide, deoxyribonucleotide, or nucleotide analog
cncagagnnncnagagaan

SEQ ID NO: 403 - DNA, 19 bp, Artificial Sequence
  based on Homo sapiens. Each nucleobase may be part of a ribonucleotide, deoxyribonucleotide, or nucleotide analog
angnncncanncgagcngc

SEQ ID NO: 404 - DNA, 19 bp, Artificial Sequence
  based on Homo sapiens. Each nucleobase may be part of a ribonucleotide, deoxyribonucleotide, or nucleotide analog
ngaacnggaacacnagang

SEQ ID NO: 405 - DNA, 19 bp, Artificial Sequence
  based on Homo sapiens. Each nucleobase may be part of a ribonucleotide, deoxyribonucleotide, or nucleotide analog
gcncaggcngaacnggaac

SEQ ID NO: 406 - DNA, 19 bp, Artificial Sequence
  based on Homo sapiens. Each nucleobase may be part of a ribonucleotide, deoxyribonucleotide, or nucleotide analog
nngacancancanngcgac

SEQ ID NO: 407 - DNA, 19 bp, Artificial Sequence
  based on Homo sapiens. Each nucleobase may be part of a ribonucleotide, deoxyribonucleotide, or nucleotide analog
accancacaacaaaagcan

SEQ ID NO: 408 - DNA, 19 bp, Artificial Sequence
  based on Homo sapiens. Each nucleobase may be part of a ribonucleotide, deoxyribonucleotide, or nucleotide analog
ccacnnggcangnncnacc

SEQ ID NO: 409 - DNA, 19 bp, Artificial Sequence
  based on Homo sapiens. Each nucleobase may be part of a ribonucleotide, deoxyribonucleotide, or nucleotide analog
ncgnancaagaacncacac

SEQ ID NO: 410 - DNA, 19 bp, Artificial Sequence
  based on Homo sapiens. Each nucleobase may be part of a ribonucleotide, deoxyribonucleotide, or nucleotide analog
ggnancngaagnngacaac

SEQ ID NO: 411 - DNA, 19 bp, Artificial Sequence
  based on Homo sapiens. Each nucleobase may be part of a ribonucleotide, deoxyribonucleotide, or nucleotide analog
nnncnncnccagnggnanc

SEQ ID NO: 412 - DNA, 19 bp, Artificial Sequence
  based on Homo sapiens. Each nucleobase may be part of a ribonucleotide, deoxyribonucleotide, or nucleotide analog
nncnccaggnccaaaanga

SEQ ID NO: 413 - DNA, 19 bp, Artificial Sequence
  based on Homo sapiens. Each nucleobase may be part of a ribonucleotide, deoxyribonucleotide, or nucleotide analog
acagcancnncngaagaac

SEQ ID NO: 414 - DNA, 19 bp, Artificial Sequence
  based on Homo sapiens. Each nucleobase may be part of a ribonucleotide, deoxyribonucleotide, or nucleotide analog
cacaggngnanncancang

SEQ ID NO: 415 - DNA, 19 bp, Artificial Sequence
  based on Homo sapiens. Each nucleobase may be part of a ribonucleotide, deoxyribonucleotide, or nucleotide analog
ccaggncncnannaaagcc

SEQ ID NO: 416 - DNA, 19 bp, Artificial Sequence
  based on Homo sapiens. Each nucleobase may be part of a ribonucleotide, deoxyribonucleotide, or nucleotide analog
nncncnccagnngncagga

SEQ ID NO: 417 - DNA, 19 bp, Artificial Sequence
  based on Homo sapiens. Each nucleobase may be part of a ribonucleotide, deoxyribonucleotide, or nucleotide analog
gaagngcngacacaananc

SEQ ID NO: 418 - DNA, 19 bp, Artificial Sequence
  based on Homo sapiens. Each nucleobase may be part of a ribonucleotide, deoxyribonucleotide, or nucleotide analog
nnnnccnncnccnccncnc

SEQ ID NO: 419 - DNA, 19 bp, Artificial Sequence
  based on Homo sapiens. Each nucleobase may be part of a ribonucleotide, deoxyribonucleotide, or nucleotide analog
cancngangccannncnnc

SEQ ID NO: 420 - DNA, 19 bp, Artificial Sequence
  based on Homo sapiens. Each nucleobase may be part of a ribonucleotide, deoxyribonucleotide, or nucleotide analog
agccanncngnncnnccga

SEQ ID NO: 421 - DNA, 19 bp, Artificial Sequence
  based on Homo sapiens. Each nucleobase may be part of a ribonucleotide, deoxyribonucleotide, or nucleotide analog
ccagganaggaagcacaca

SEQ ID NO: 422 - DNA, 19 bp, Artificial Sequence
  based on Homo sapiens. Each nucleobase may be part of a ribonucleotide, deoxyribonucleotide, or nucleotide analog
anggnancaancagnncnc

SEQ ID NO: 423 - DNA, 19 bp, Artificial Sequence
  based on Homo sapiens. Each nucleobase may be part of a ribonucleotide, deoxyribonucleotide, or nucleotide analog
ccgcagcannnccnnnaac

SEQ ID NO: 424 - DNA, 19 bp, Artificial Sequence
  based on Homo sapiens. Each nucleobase may be part of a ribonucleotide, deoxyribonucleotide, or nucleotide analog
cagnnnnngaagangnngg

SEQ ID NO: 425 - DNA, 19 bp, Artificial Sequence
  based on Homo sapiens. Each nucleobase may be part of a ribonucleotide, deoxyribonucleotide, or nucleotide analog
gngacagaccngaaacanc

SEQ ID NO: 426 - DNA, 19 bp, Artificial Sequence
  based on Homo sapiens. Each nucleobase may be part of a ribonucleotide, deoxyribonucleotide, or nucleotide analog
gggcannnncnnagagaag

SEQ ID NO: 427 - DNA, 19 bp, Artificial Sequence
  based on Homo sapiens. Each nucleobase may be part of a ribonucleotide, deoxyribonucleotide, or nucleotide analog
agnacccnngannanaccc

SEQ ID NO: 428 - DNA, 19 bp, Artificial Sequence
  based on Homo sapiens. Each nucleobase may be part of a ribonucleotide, deoxyribonucleotide, or nucleotide analog
gaaangnacgaacagnacc

SEQ ID NO: 429 - DNA, 19 bp, Artificial Sequence
  based on Homo sapiens. Each nucleobase may be part of a ribonucleotide, deoxyribonucleotide, or nucleotide analog
ngaaaaacncanaannccc

SEQ ID NO: 430 - DNA, 19 bp, Artificial Sequence
  based on Homo sapiens. Each nucleobase may be part of a ribonucleotide, deoxyribonucleotide, or nucleotide analog
ccancnnnncagaaacaag

SEQ ID NO: 431 - DNA, 19 bp, Artificial Sequence
  based on Homo sapiens. Each nucleobase may be part of a ribonucleotide, deoxyribonucleotide, or nucleotide analog
cnanaanncncnccagnng

SEQ ID NO: 432 - DNA, 19 bp, Artificial Sequence
  based on Homo sapiens. Each nucleobase may be part of a ribonucleotide, deoxyribonucleotide, or nucleotide analog
cncccnnaggnacacanac

SEQ ID NO: 433 - DNA, 19 bp, Artificial Sequence
  based on Homo sapiens. Each nucleobase may be part of a ribonucleotide, deoxyribonucleotide, or nucleotide analog
acaagcagngacacnacnc

SEQ ID NO: 434 - DNA, 19 bp, Artificial Sequence
  based on Homo sapiens. Each nucleobase may be part of a ribonucleotide, deoxyribonucleotide, or nucleotide analog
gnaacnccngaaangangc

SEQ ID NO: 435 - DNA, 19 bp, Artificial Sequence
  based on Homo sapiens. Each nucleobase may be part of a ribonucleotide, deoxyribonucleotide, or nucleotide analog
caacaaanccagnaacncc

SEQ ID NO: 436 - DNA, 19 bp, Artificial Sequence
  based on Homo sapiens. Each nucleobase may be part of a ribonucleotide, deoxyribonucleotide, or nucleotide analog
caccanaacncngangaac

SEQ ID NO: 437 - DNA, 20 bp, Artificial Sequence
  based on Homo sapiens
naggacnngnccaccnnnnc

SEQ ID NO: 438 - DNA, 20 bp, Artificial Sequence
  based on Homo sapiens
nngaaaanaggacnngncca

SEQ ID NO: 439 - DNA, 20 bp, Artificial Sequence
  based on Homo sapiens
ncnncncnngaaaanaggac

SEQ ID NO: 440 - DNA, 20 bp, Artificial Sequence
  based on Homo sapiens
cancnncncnngaaaanagg

SEQ ID NO: 441 - DNA, 20 bp, Artificial Sequence
  based on Homo sapiens
gncancnncncnngaaaana

SEQ ID NO: 442 - DNA, 20 bp, Artificial Sequence
  based on Homo sapiens
aagncancnncncnngaaaa

SEQ ID NO: 443 - DNA, 20 bp, Artificial Sequence
  based on Homo sapiens
aaaagncancnncncnngaa

SEQ ID NO: 444 - DNA, 20 bp, Artificial Sequence
  based on Homo sapiens
nnaaaagncancnncncnng

SEQ ID NO: 445 - DNA, 20 bp, Artificial Sequence
  based on Homo sapiens
ngnnaaaagncancnncncn

SEQ ID NO: 446 - DNA, 20 bp, Artificial Sequence
  based on Homo sapiens
acngnnaaaagncancnncn

SEQ ID NO: 447 - DNA, 20 bp, Artificial Sequence
  based on Homo sapiens
aaacngnnaaaagncancnn

SEQ ID NO: 448 - DNA, 20 bp, Artificial Sequence
  based on Homo sapiens
caaaacngnnaaaagncanc

SEQ ID NO: 449 - DNA, 20 bp, Artificial Sequence
  based on Homo sapiens
nncaaaacngnnaaaagnca

SEQ ID NO: 450 - DNA, 20 bp, Artificial Sequence
  based on Homo sapiens
gangncngcaggnacacaag

SEQ ID NO: 451 - DNA, 20 bp, Artificial Sequence
  based on Homo sapiens
nagcaaaagnnnnnaancna

SEQ ID NO: 452 - DNA, 20 bp, Artificial Sequence
  based on Homo sapiens
gcangacaacnaaagcaccg

SEQ ID NO: 453 - DNA, 20 bp, Artificial Sequence
  based on Homo sapiens
aancngcaannngggganac

SEQ ID NO: 454 - DNA, 20 bp, Artificial Sequence
  based on Homo sapiens
nngnacngaccanncnggan

SEQ ID NO: 455 - DNA, 20 bp, Artificial Sequence
  based on Homo sapiens
ncngcangngncncagangg

SEQ ID NO: 456 - DNA, 20 bp, Artificial Sequence
  based on Homo sapiens
acaanacanggcagggnncc

SEQ ID NO: 457 - DNA, 20 bp, Artificial Sequence
  based on Homo sapiens
ngccnacnanagagnnagan

SEQ ID NO: 458 - DNA, 20 bp, Artificial Sequence
  based on Homo sapiens
naanggaanncaanccngan

SEQ ID NO: 459 - DNA, 20 bp, Artificial Sequence
  based on Homo sapiens
caacnaaaacacngccangn

SEQ ID NO: 460 - DNA, 20 bp, Artificial Sequence
  based on Homo sapiens
nangangcnncnnanncnna

SEQ ID NO: 461 - DNA, 20 bp, Artificial Sequence
  based on Homo sapiens
annngnnaagccnancngaa

SEQ ID NO: 462 - DNA, 20 bp, Artificial Sequence
  based on Homo sapiens
nccaccagcanggaacaann

SEQ ID NO: 463 - DNA, 20 bp, Artificial Sequence
  based on Homo sapiens
agaaaanggacagaanccna

SEQ ID NO: 464 - DNA, 20 bp, Artificial Sequence
  based on Homo sapiens
cnancannaaanacgcnnnc

SEQ ID NO: 465 - DNA, 20 bp, Artificial Sequence
  based on Homo sapiens
nannaacaacanacanacnn

SEQ ID NO: 466 - DNA, 20 bp, Artificial Sequence
  based on Homo sapiens
ggnnaggnnacngangnnag

SEQ ID NO: 467 - DNA, 18 bp, Artificial Sequence
  based on Homo sapiens
nnaccanaggncccagnc

SEQ ID NO: 468 - DNA, 19 bp, Artificial Sequence
  based on Homo sapiens
nnnaccanaggncccagnc

(19) United States
(12) Patent Application Publication
(10) Pub. No.: US 2008/0293653 A1
(43) Pub. Date: Nov. 27, 2008

(54) Method of treating autoimmune diseases

(76) Inventors: Jon Durkin, Ottawa (CA); John Gillard, Baie d'Urfe (CA); Trevor Owens, Odense M (DK); Stephen Morris, Beaconsfield (CA); Simone Patricia Zehnter, Montreal (CA)

Correspondence Address: PHILIP SWAIN, PHD;C/O GOWLING LAFLEUR HENDERSON, 1 PLACE VILLE MARIE,, 37TH FLOOR, MONTREAL, QC, H3B 3P4, CA

(21) Appl. No.: 11/247,331

(22) Filed: Oct. 12, 2005

Related U.S. Application Data
(60) Provisional application No. 60/618,891, filed on Oct. 14, 2004.

(51) Int. Cl. A61K 31/70; C12N 5/06; C12Q 1/68; C12Q 1/02; A61P 37/00

(52) U.S. Cl. 514/44; 435/375; 435/6; 435/29

(57) ABSTRACT

The invention features a method of inducing an apoptosis-resistant cell to undergo apoptosis, in which the cell is associated with an autoimmune disease such as multiple sclerosis. The method involves sensitizing the cell to apoptosis stimuli by treating the cell with an IAP antisense oligonucleotide, so that the cell undergoes apoptosis at a site of autoimmune disease.

### CROSS-REFERENCE TO RELATED APPLICATIONS

This application claims priority to and benefit of previously filed U.S. provisional application Ser. No. 60/618,891, filed Oct. 14, 2004, the entire contents of which are hereby incorporated by reference.

### FIELD OF THE INVENTION

The present invention concerns methods of treating autoimmune diseases, and more particularly methods of treating autoimmune diseases characterized by apoptosis-resistant cells.

### BACKGROUND OF THE INVENTION

The systematic elimination of T-cells by apoptosis is a key factor in the maintenance of a normal, healthy immune system and prevention of autoimmune diseases. Recent studies have revealed a defect in apoptotic T-cell death in autoimmune disease, which implicates the inhibitors of apoptosis protein (IAP), including XIAP, in abnormal resistance to apoptotic stimuli. Comi, C., M. Leone, et al. (2000) in “Defective T-cell fas function in patients with multiple sclerosis” Neurology 55(7), and Zipp F. (2000) “Apoptosis in multiple sclerosis.” Cell Tissue Res. 301(1):163-71 disclose that patients with multiple sclerosis (MS) have defects in the coupling of the death receptor, Fas, in T lymphocytes to the downstream caspase-3. IAP proteins may play a crucial role in the pathogeneisis of MS by blocking the execution of the normal apoptotic default of activated T-cells, as disclosed by Sharief, M. K. and Y. K. Semra (2001) in “Upregulation of the inhibitor of apoptosis proteins in activated T lymphocytes from patients with multiple sclerosis.” J Neuroimmunol 119(2):350-7. Sharief M. K., M. A. Noori et al. (2002) in “Reduced expression of the inhibitor of apoptosis proteins in T-cells from patients with multiple sclerosis following interferon-beta therapy” J. Neuroimmunol 129(1-2):224-31 disclosed that the expression levels of IAP proteins were significantly increased in mitogen stimulated T lymphocytes from MS patients. The elevated expression of IAPs correlates with MS disease activity and with T lymphocyte resistance to apoptosis as demonstrated by Semra, Y. K., O. A. Seidi, et al. (2002) in “Disease activity in multiple sclerosis correlates with T lymphocyte expression of the inhibitor of apoptosis proteins.” J Neuroimmunol 122(1-2):159-66.

Current therapies for autoimmune disease lack efficacy and are often associated with considerable patient discomfort. Studies suggest that strategies to decrease IAP expression levels may be clinically useful in the treatment of autoimmune disease such as MS. Conte, D. P. Liston, et al (2001) in “Thymocyte-targeted overexpression of XIAP transgene disrupts T lymphoid apoptosis and maturation” Proc Natl Acad Sci USA 98(9):5049-54 showed that XIAP levels regulate T lymphocyte sensitivity to apoptotic stimuli. Sharief, M. K., M. A. Noori et al (2002) in “Reduced expression of the inhibitor of apoptosis proteins in T-cells from patients with multiple sclerosis following interferon-beta therapy.” J Neuroimmunol 129(1-2):224-31 demonstrated that efficacious treatment of MS patients with interferon-beta correlated with deceased expression of IAP proteins and with increased susceptibility of T-cells to apoptosis.

T lymphocytes and other cells, which are resistant to apoptosis, and which are characteristic of certain autoimmune diseases, are an attractive target for therapeutic intervention. This prompted us to use animal models of human autoimmune diseases to further investigate the relationship between XIAP levels and cell sensitivity to apoptosis stimuli with a view to developing a novel approach to treating such diseases.

### SUMMARY OF THE INVENTION

We have made a new and entirely unexpected discovery that in experimental autoimmune encephalitis (EAE), a mouse model for human multiple sclerosis (MS), a XIAP antisense oligonucleotide increases apoptosis of T-cells after the T-cells infiltrate the Central Nervous System (CNS). In a population of mice pre-treated with the XIAP antisense oligonucleotide, a significant number of the mice did not develop symptoms of EAE. A population of mice, which were treated with the XIAP antisense oligonucleotide at initiation of EAE symptoms resulted in a significant number of the mice having significantly reduced symptoms of EAE. Surprisingly, in both cases of treated mice, T-cells were found to be undergoing apoptosis in the CNS, specifically the spinal cord, which is contrary to previous observations that autoactivated T-cells, when infiltrated into the CNS, resulted in animals having symptoms of EAE. Advantageously, our discovery has far reaching implications in developing antisense therapies for treating autoimmune diseases, such as multiple sclerosis or Crohn's disease, which are characterized by T-cells that are resistant to apoptosis. Moreover, autoimmune diseases that are characterized by other types of apoptosis resistant cells, such as keratinocytes and synoviocytes in psoriasis and rheumatoid arthritis respectively, may also be treatable using the methods and compositions of the present invention, thereby addressing a significant unmet medical need. An additional advantage is that accelerated apoptosis at the site of inflammation, as opposed to in lymphoid tissue, may likely increase the specificity of the autoimmune disease treatment. Because T-cells are sensitized for apoptosis in the periphery before accessing the site of inflammation, there would likely be no requirement for the drug to access or accumulate at the site of inflammation. Therefore, drug induced adverse effects at tissues, such as the CNS, may be significantly reduced or essentially eliminated.

The present invention therefore provides a method of treating multiple sclerosis, using prophylactic and therapeutic compositions of XIAP antisense oligonucleotides that sensitize T-cells to apoptosis stimuli so that they undergo apoptosis at the site of autoimmune disease. The compositions of the present invention may be capable of stimulating T-cells to undergo apoptosis in a population of humans suffering from, or having a predisposition towards, multiple sclerosis, so that the symptoms of the disease are significantly reduced or essentially eliminated, or reversed. Furthermore, as demonstrated herein, the onset or progression of the disease symptoms is significantly reduced, slowed or essentially eliminated. Moreover, the compositions of the present invention when administered at presentation of the disease, provide long-term resolution of paralysis or other signs of the disease. In addition, the methods and compositions of the present invention are compatible with currently used MS therapies that are known to those skilled in the art, such as, but not limited to, β-interferon and corticosteriods.

Accordingly in one embodiment of the present invention, there is provided a method of inducing an apoptosis-resistant cell to undergo apoptosis, the cell being associated with an autoimmune disease, the method comprising: sensitizing the apoptosis-resistant cell to apoptosis stimuli by treating the cell with an IAP antisense oligonucleotide, so that the cell undergoes apoptosis at a site of autoimmune disease. In one aspect of the present invention, the IAP antisense oligonucleotide comprises eight or more and thirty or less consecutive nucleobases in length. In one example, the IAP antisense oligonucleotide is a XIAP antisense oligonucleotide. In another example, the IAP antisense oligonucleotide is a HIAP1 antisense oligonucleotide. In yet another example, the IAP antisense oligonucleotide is a HIAP2 antisense oligonucleotide.

In another aspect of the present invention, the IAP antisense oligonucleotide comprises eight or more nucleobases of a sequence selected from the group consisting of: at least one of SEQ ID NOs: 1-466. In one example, the IAP antisense oligonucleotide comprises eight or more nucleobases of a sequence selected from the group consisting of: at least one of SEQ ID NOs: 1-96, and 195-275. In another example, the IAP antisense oligonucleotide consists of a sequence selected from the group consisting of: at least one of SEQ ID NOs: 31, 41, 47, 93, 195, 196, 197, 241, 245, 249, 270, and 272. In another example, the IAP antisense oligonucleotide comprises eight or more nucleobases of a sequence selected from the group consisting of: at least one of SEQ ID NOs: 97-194, and 276-365. In yet another example, the IAP antisense oligonucleotide comprises eight or more nucleobases of a sequence selected from the group consisting of: at least one of SEQ ID NOs: 366-436. In another aspect of the present invention, the apoptosis resistant cell is a T-cell, a synoviocyte, or a keratinocyte. In one example, the apoptosis resistant cell is a T-cell., specifically a CD4⁺ T-cell. In yet another aspect of the present invention, the site of autoimmune disease is brain, myelin, intestinal mucosa, skin, or synovium. In one example, the site of autoimmune disease is brain or myelin.

In another aspect of the present invention, the autoimmune disease is EAE, multiple sclerosis, Crohn's disease, lupus erythematosus, rheumatoid arthritis, osteoarthritis, psoriasis, ulcerative colitis, type I diabetes, pancreatitis, asthma, idiopathic thrombocytopenia purpura, uveitis, Guillain-Barre syndrome or myasthenia gravis. In one example, the autoimmune disease is multiple sclerosis.

According to another embodiment of the present invention, there is provided a method of treating an autoimmune disease, the disease being characterized by apoptosis-resistant cells, the method comprising: administering to a mammalian subject in need thereof an IAP antisense oligonucleotide in a pharmaceutically acceptable carrier to sensitize the apoptosis-resistant cells to apoptosis stimuli, so that the cells undergo apoptosis at a site of autoimmune disease, thereby treating the disease. In one aspect, the autoimmune disease is EAE, multiple sclerosis, Crohn's disease, lupus erythematosus, rheumatoid arthritis, osteoarthritis, psoriasis; ulcerative colitis, type I diabetes, pancreatitis, asthma, idiopathic thrombocytopenia purpura, uveitis, Guillain-Barre syndrome or myasthenia gravis. In one example, the autoimmune disease is multiple sclerosis. In another example, the autoimmune disease is rheumatoid arthritis.

In one aspect, the mammalian subject is a mouse, a human, a rat, a primate, or a guinea pig. In one example, the mammalian subject is a human.

In another embodiment of the present invention, there is provided a method of inducing apoptosis in an apoptosis-resistant cell, the cell being associated with an autoimmune disease, the method comprising: sensitizing the apoptosis-resistant cell to apoptosis stimuli by treating the cell with a XIAP antisense oligonucleotide comprising eight or more and thirty or less consecutive nucleobases in length, so that the cell undergoes apoptosis at a site of autoimmune disease. In one example, the XIAP antisense oligonucleotide comprises eight or more nucleobases of a sequence selected from the group consisting of: at least one of SEQ ID NOs: 1-96, and 195-275. In another example, the XIAP antisense oligonucleotide consist of a sequence selected from the group consisting of: at least one of SEQ ID NOs: 31, 41, 47, 93, 195, 196, 197, 241, 245, 249, 270, and 272.

According to yet another embodiment of the present invention, there is provided a method of treating a CNS inflammatory autoimmune disease characterized by apoptosis-resistant T-cells, the method comprising: administering to a mammalian subject a XIAP antisense oligonucleotide in a pharmaceutically acceptable carrier to sensitize the apoptosis-resistant T-cells to apoptosis stimuli, so that the T-cells undergo apoptosis at a site of CNS inflammatory autoimmune disease, thereby treating the disease. In one aspect, the site of the CNS inflammatory autoimmune disease is brain or myelin. In one example, the CNS inflammatory disease is multiple sclerosis.

According to still another embodiment of the present invention, there is provided a method of treating multiple sclerosis in a human, the method comprising: administering to the human a XIAP antisense oligonucleotide in a pharmaceutically acceptable carrier to sensitize apoptosis-resistant T-cells to apoptosis stimuli, so that the T-cells undergo apoptosis at the CNS, thereby treating the multiple sclerosis.

According to another embodiment of the present invention, there is provided a method of alleviating the symptoms of multiple sclerosis in a human, the method comprising: administering to the human a XIAP antisense oligonucleotide in a pharmaceutically acceptable carrier to sensitize apoptosis-resistant T-cells to apoptosis stimuli, so that the T-cells undergo apoptosis at the CNS, thereby alleviating the symptoms of multiple sclerosis.

According to still another embodiment of the present invention there is provided a method of preventing the onset of multiple sclerosis in a human, the method comprising: administering to the human a XIAP antisense oligonucleotide in a pharmaceutically acceptable carrier to sensitize apoptosis-resistant T-cells to apoptosis stimuli, so that the T-cells undergo apoptosis at the CNS, thereby preventing the onset of multiple sclerosis in the human.

According to another embodiment of the present invention, there is provided a method of predicting a patient's suitability for therapy, the method comprising:
a) isolating apoptosis-resistant cells from a blood sample taken from a patient suffering from an autoimmune disease characterized by apoptosis-resistant cells;
b) contacting the apoptosis-resistant cells with an IAP antisense oligonucleotide;
c) adding apoptosis stimuli to the contacted cells of step b); and
d) measuring apoptosis of the cells, apoptosed cells indicating that treatment with the IAP antisense oligonucleotide is suitable for the patient.

According to another embodiment of the present invention, there is provided an in vivo assay for identifying a compound that sensitizes an apoptosis-resistant cell to apoptosis stimuli, the method comprising:
a) peripherally administering a test compound to a non-human mammal suffering from an autoimmune disease characterized by apoptosis-resistant cells; and
b) analyzing a sample of blood or tissue for increased cell apoptosis taken from the mammal, an increase in cell apoptosis being an indication that the test compound increases the sensitivity of the cell to apoptosis stimuli at a site of autoimmune disease.

According to yet another embodiment of the present invention, there is provided an in vivo assay for identifying a compound that sensitizes an apoptosis-resistant cell to apoptosis stimuli, the method comprising:
a) administering a test compound to a site of autoimmune disease in a non-human mammal suffering from an autoimmune disease characterized by apoptosis-resistant cells; and
b) analyzing a sample of tissue taken from the site of autoimmune disease for increased cell apoptosis, an increase in cell apoptosis being an indication that the test compound increases the sensitivity of the cell to apoptosis stimuli at the site of the autoimmune disease.

According to another embodiment of the present invention, there is provided a pharmaceutical composition, the composition comprising: an IAP antisense oligonucleotide in a pharmaceutically acceptable carrier, the oligonucleotide being in sufficient quantity to sensitize apoptosis-resistant cells to apoptosis stimuli so that the cells undergo apoptosis at a site of autoimmune disease, the disease being characterized by apoptosis-resistant cells. In one aspect, the IAP antisense oligonucleotide comprises eight or more and thirty or less consecutive nucleobases in length. In one example, the IAP antisense oligonucleotide is a XIAP antisense oligonucleotide. In another example, the IAP antisense oligonucleotide is a HIAP1 antisense oligonucleotide. In another example, the IAP antisense oligonucleotide is a HIAP2 antisense oligonucleotide. In yet another example, the IAP antisense oligonucleotide comprises eight or more nucleobases of a sequence selected from the group consisting of: at least one of SEQ ID NOs: 1-466. In another example, the IAP antisense oligonucleotide comprises eight or more nucleobases of a sequence selected from the group consisting of: at least one of SEQ ID NOs: 1-96, and 195-275. In yet another example, the IAP antisense oligonucleotide consist of a sequence selected from the group consisting of: at least one of SEQ ID NOs: 31, 41, 47, 93, 195, 196, 197, 241, 245, 249, 270, and 272. In still another example, the IAP antisense oligonucleotide comprises eight or more nucleobases of a sequence selected from the group consisting of: at least one of SEQ ID NOs: 97-194, and 276-365.

In another example, the IAP antisense oligonucleotide comprises eight or more nucleobases of a sequence selected from the group consisting of: at least one of SEQ ID NOs: 366-436.

According to one embodiment of the present invention, there is provided a method of treating arthritis in a human, the method comprising: administering to the human a XIAP antisense oligonucleotide in a pharmaceutically acceptable carrier to sensitize apoptosis-resistant leukocytes or synoviocytes to apoptosis stimuli so that the leukocytes or synoviocytes undergo apoptosis at the synovium, thereby treating the arthritis.

According to another embodiment of the present invention, there is provided a method of treating Crohn's Disease in a human, the method comprising: administering to the human a XIAP antisense oligonucleotide in a pharmaceutically acceptable carrier to sensitize apoptosis-resistant T-cells to apoptosis stimuli so that the T-cells undergo apoptosis at the intestinal mucosa, thereby treating the Crohn's Disease.

According to still another embodiment of the present invention, there is provided a method of treating psoriasis in a human, the method comprising: administering to the human a XIAP antisense oligonucleotide in a pharmaceutically acceptable carrier to sensitize apoptosis-resistant T-cells and keratinocytes to apoptosis stimuli so that the T-cells and keratinocytes undergo apoptosis at the skin, thereby treating the psoriasis.

According to another embodiment of the present invention, there is provided a process for making a compound that increases the sensitivity of an apoptosis-resistant cell to apoptosis stimuli, the process comprising:
a) carrying out any of the screening methods described herein to identify a compound that increases the sensitivity of the cell to apoptosis stimuli at a site of autoimmune disease; and
b) manufacturing the compound.

According to another embodiment of the present invention, there is provided a kit comprising:
a) a vessel or vessels containing purified apoptosis stimuli and a purified IAP antisense oligonucleotide; and
b) instructions for drawing blood or a tissue sample from a subject and for mixing the blood with the oligonucleotide and the apoptosis stimuli.

According to another embodiment of the present invention, there is provided a kit comprising:
a) a vessel or vessels containing purified apoptosis stimuli and a purified IAP antisense oligonucleotide;
b) a needle for drawing blood or a tissue sample; and
c) instructions for drawing blood or a tissue sample from a subject and for mixing the blood or the tissue sample with the oligonucleotide and the apoptosis stimuli.

According to another embodiment of the present invention, there is provided an article of manufacture comprising:
a) a vial containing purified apoptosis stimuli and a purified IAP antisense oligonucleotide; or
b) packaged together, a first vial containing purified apoptosis stimuli and a second vial containing a purified IAP antisense oligonucleotide; and
c) instructions for drawing blood or a tissue sample from a subject and for mixing the blood or the tissue sample with the oligonucleotide and the apoptosis stimuli.

Accordingly in an alternative aspect of the present invention, there is provided a method of inducing apoptosis, the method comprising: sensitizing to apoptosis stimuli at least one apoptosis-resistant cell in a population of cells, the cell being contacted with an IAP antisense oligonucleotide so that the cell undergoes apoptosis at its target site.

The IAP antisense oligonucleotide molecules that are useful in practicing the present invention are SEQ ID NOs: 1 through 466 disclosed in Tables 1 through 7 below.

### BRIEF DESCRIPTION OF THE DRAWINGS

Further aspects and advantages of the present invention will become better understood with reference to the description in association with the following Figures, wherein:

FIG. 1 is a graphical illustration of EAE scores mice prophylactically treated with a human/murine specific XIAP antisense (SEQ ID NO: 41), control oligonucleotide (SEQ ID NO: 468), and mismatch sequence (SEQ ID NO: 467), and saline;

FIG. 2 is a graphical representation of lymphocyte proliferation in response to MOG and a control peptide antigen challenge showing that pre-treatment with antisense oligonucleotide is not immunosuppressive;

FIG. 3 Illustrates flow cytometry analysis of perfused mouse CNS showing CNS infiltrate;

FIG. 4 is a graphical illustration of therapeutic treatment of EAE using antisense therapy. Antisense therapy began at day 0 after mice presented symptoms;

FIG. 5 is a graphical representation showing reduced microglial activation in XIAP antisense protected mice;

FIG. 6 illustrates CNS histology of XIAP antisense treated mice following EAE;

FIG. 7 illustrates CD4 and CD11/Mac-1 analysis of CNS after inhibition of EAE (A,B,C,D) and semi-quantitative analysis of number of CNS-infiltrating cells (E);

FIG. 8 illustrates apoptotic cells in XIAP antisense treated CNS were lymphocytes not neurons. Apoptotic cells (TdT positive) are largely confined to Icam positive regions of infiltrate and when present in the grey matter do not colocalize with a neuronal marker (Neu N);

FIG. 9 illustrates CNS histology of control antisense treated mice following EAE;

FIG. 10 illustrates Tunel and CD4 analysis of CNS of mice that were treated with control antisense. Tunel positive (green) CD4+ cells (red) were observed in SEQ ID NO:41 treated CNS (A). CD4 positive cells were reduced in CNS of SEQ ID NO:41 treated mice (B) compared to control (C). Cells from multiple sections were quantified (D);

FIG. 11 is a graphical representation of ex-vivo activation induced cell death of CD4+ve T-cells from saline or SEQ ID NO: 41 treated mice;

FIG. 12 is a graphical representation of mean clinical scores of collagen-induced arthritis in control and XIAP antisense treated mice;

FIG. 13 is a graphical representation of mean histological scores of collagen-induced arthritis pathology in fore and hind paws of control and XIAP antisense treated mice;

FIG. 14 is a graphical representation of mean histological scores of collagen-induced arthritis pathology in the knees of control and XIAP antisense treated mice; and

FIG. 15 is a graphical representation of the summed mean histological scores of collagen-induced arthritis pathology in the knees and paws of control and XIAP antisense treated mice.

### DETAILED DESCRIPTION OF THE INVENTION

### Definitions

Unless otherwise stated, the following terms apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein the terms “apoptosis” is intended to mean the process of cell death in which a dying cell displays a set of well-characterized biochemical indicia that include cell membrane blebbing, cell soma shrinkage, chromatin condensation, and DNA fragmentation.

As used herein, the term “apoptosis resistant”, when referring to cells, specifically T-cells, synoviocytes, keratinocytes and the like, is intended to mean that an increased amount of apoptosis stimuli is required to cause apoptosis in cells that otherwise fail to die by apoptosis.

As used herein, the terms “oligonucleotide” and “nucleobase oligomers” are used interchangeably and are intended to mean a compound that includes a chain of eight or more and thirty or less consecutive nucleobases (or nucleotides) in length joined together by linkage groups. Included in this definition are i) natural and ii) non-natural oligonucleotides, both modified and unmodified, as well as oligonucleotide phosphate ester linkage mimetics such as Peptide Nucleic Acids (PNAs), phosphomorpholino nucleic acids (PMO), locked nucleic acids (LNA) and arabinonucleic acids (ANA).

Numerous oligonucleotides and nucleobase oligomers which can be used to practice the methods of the present invention are described in detail under the section “Compositions and administration” below.

As used herein, the term “hybridization” is intended to mean hydrogen bonding, which may be Watson-Crick Hoogsteen or reversed Hoogsteen hydrogen bonding, between complimentary nucleobases. For example, adenine and thymine are complementary nucleobases that pair through the formation of hydrogen bonds.

As used herein, the term “autoimmune disease” is intended to mean a disorder in which there is an immune response to self antigen, which is characterized by some cells, which are resistant to, and fail to die, by apoptosis. Examples of autoimmune diseases include, but are not limited to, multiple sclerosis, Crohn's disease, lupus erythematosus, rheumatoid arthritis, osteoarthritis, psoriasis, ulcerative colitis, type I diabetes, pancreatitis, asthma, idiopathic thrombocytopenia purpura, uveitis, Guillain-Barre syndrome and myasthenia gravis.

As used herein, the term “cell” is intended to mean a single-cellular organism, a cell from a multi-cellular organism or it may be a cell contained in a multi-cellular organism.

As used herein, the term “sensitize the cell to apoptosis stimuli” when referring to IAP antisense oligonucleotide treatment, is intended to mean that the IAP antisense oligonucleotide sensitizes a cell, specifically a T-cell, to apoptose after a challenge with an autoantigen, such as MOG or a T-cell epitope containing peptide derived from MOG or an endogenous apoptotic stimuli such as Fas ligand or and exogenous stimuli such as methotrexate. A T-cell epitope is the smallest unit of recognition by a T-cell receptor in which the epitope includes amino acids essential to receptor recognition. T-cell epitopes are believed to be involved in the initiation and perpetuation of an immune response to an antigen or an autoantigen. The T-cell epitopes are thought to trigger an early immune response of T helper cells by binding to an appropriate HLA molecule on an antigen presenting cell and stimulating the relevant T-cell subpopulation. This leads to T-cell proliferation, lymphokine secretion, local inflammatory reactions, recruitment of additional cells to the site and activation of B cells.

As used herein, the term “apoptosis stimuli” is intended to mean activators of a cell death receptor such as Fas ligand, TRAIL, TNF-α, TNF-β, and the like. Other apoptosis stimuli include, for example, chemotherapeutic agents such as etoposide.

As used herein, the term “inducing an apoptosis-resistant cell to undergo apoptosis” is intended to mean causing the number of cells that are apoptosis resistant (e.g. T-cells or any other cells) in a given cell population, to apoptose. It will be appreciated by one skilled in the art that the degree of apoptosis inducement provided by an apoptosis inducing IAP antisense oligonucleotide or test compound will vary in a given treatment or given assay. One skilled in the art can determine the statistically significant change in the level of apoptosis that identifies a nucleobase oligomers that induces apoptosis otherwise limited by an IAP. Preferably, “inducing apoptosis” means the number of apoptosis resistant cells undergoing apoptosis is at least 10% or more relative to cells not resistant to apoptosis.

As used herein, the term “RNAi” is intended to mean RNA duplexes (double stranded RNA or dsRNA) designed to cause RNA degradation through the dicer complex.

As used herein, the term “subject” or “patient” is used interchangeably and is intended to mean mammals such as humans, primates, rats, mice, guinea pigs, goats, sheep, horses, pigs and the like.

As used herein, the term “CNS inflammatory disease” is intended to mean a disease that is characterized by inflammation of tissues of the CNS, such as brain, spinal cord, optic nerve, and the like, including but not limited to multiple sclerosis.

As used herein, the term “treating multiple sclerosis (MS)” is intended to mean prophylactic treatment of mammals, including humans, which are susceptible to MS; treating the initial onset of MS; and treating advanced stage MS. The term “advanced stage” is intended to mean relapsing-remitting MS, chronic progressive MS, primary progressive MS and benign MS.

As used herein, the term “target site” or “site of autoimmune disease” are used interchangeably and is intended to mean the self antigen, which the apoptosis resistant cells target to induce an autoimmune disease. Examples of target sites include, in the case of EAE and MS, the brain and spinal cord; in the case of Crohn's disease, the intestinal mucosa; in the case of psoriasis, the skin; and in the case of rheumatoid arthritis, the synovium.

As used herein, the term “IAP gene” is intended to mean a gene encoding a polypeptide having at least one BIR domain and which is capable of modulating (inhibiting or enhancing) apoptosis in a cell or tissue. The IAP gene is a gene having about 50% or greater nucleotide sequence identity to at least one of NAIP (Birc 1), HIAP-1 (cIAP2, API2, MIHC, hITA), HIAP-2 (cIAP1, HIHB), XIAP (hILP, hILP1, MIHA, API3), survivin (TIAP, MIHD, API4), livin (KIAP, ML-IAP, cIAP3, HIAP3), and BRUCE. The region of sequence over which identity is measured is a region encoding at least one BIR domain and a ring zinc finger domain. Mammalian IAP genes include nucleotide sequences isolated from any mammalian source. Preferably the mammal is a human.

As used herein, the term “protein”, “polypeptide” or “polypeptide fragment” is intended to mean any chain of more than two amino acids, regardless of post-translational modification, for example, glycosylation or phosphorylation, constituting all or part of a naturally occurring polypeptide or peptide, or constituting a non-naturally occurring polypeptide or peptide.

As used herein, the term “IAP protein” or “IAP polypeptide” is intended to mean a polypeptide or protein, or fragment thereof, encoded by an IAP gene. Examples of IAP polypeptides include, but are not limited to NAIP (Birc 1), HIAP-1 (cIAP2, API2, MIHC, hITA), HIAP-2 (cIAP1, HIHB), XIAP (hILP, hILP1, MIHA, API3), survivin (TIAP, MIHD, API4), livin (KIAP, ML-IAP, cIAP3, HIAP3), and BRUCE.

As used herein, the term “IAP protein function” is intended to mean any activity known to be caused in vivo or in vitro by an IAP polypeptide or IAP protein.

### I. Methods

Broadly speaking, the present invention provides a method of inducing apoptosis-resistant cells to undergo apoptosis once they are treated with an IAP antisense oligonucleotide, as described below. The cells, which are associated with an autoimmune disease characterized by cells that are resistant to apoptosis, once treated with the antisense oligonucleotide become sensitized to apoptosis stimuli so that they undergo apoptosis at the site of autoimmune disease.

The principal of antisense effects described herein are independent of sequence. It is well understood that antisense oligonucleotides can cause downregulation of target mRNA. Because of base sequence variation some antisense sequences are more active in specific species. It is clear that demonstration of efficacy in an animal model with one antisense oligonucleotide against a specific target predicts that other antisense oligonucleotide sequences can be found which will be effective in other species that express orthologous mRNA sequences.

The XIAP, HIAP1/2 antisense oligonucleotides described herein with specificity for orthologous mRNA sequences can be designed because there are regions of identity in the sequence such that XIAP, HIAP1/2 antisense sequences can be chosen that bind to, and cause down regulation of, XIAP, HIAP1/2 mRNA from multiple species. In this way it is possible to design IAP antisense oligonucleotides that are functional in both mouse and human. These antisense oligonucleotides will be efficacious in mouse models of autoimmune disease and this directly predicts that they and related antisense will be effective in the treatment of human disease. Thus the methods described herein using XIAP antisense oligonucleotides may be used to prevent, treat, ameliorate, improve, or reduce progression of autoimmune diseases characterized by apoptosis-resistant cells.

A mouse model for human MS, experimental allergic encephalomyelitis (EAE), was used to demonstrate induction of T-cell apoptosis. EAE is an inflammatory demyelinating disease of the central nervous system (CNS), which can be induced in susceptible strains of mice by immunization with MOG (myelin oligodendrocyte protein), MBP (myelin basic protein), PLP (proteolipid protein) or peptides derived therefrom. EAE is a CD4+ T-cell mediated autoimmune disease that resembles MS in some of its clinical and histological characteristics. Typically, the T-cells' target site of action in EAE and MS is the CNS. In EAE, the presence of CNS infiltrating T-cells has been thought to correlate with EAE symptoms. However, the observation that sensitized apoptosis-resistant T-cells cross the blood brain barrier and undergo rapid apoptosis in the CNS before exerting their effector function is an entirely unexpected observation in the EAE model.

Generally speaking, the IAP antisense oligonucleotides sensitize the T-cells to apoptosis stimuli after absorption while the cells are in the peripheral tissues, such as the blood, lymph nodes, spleen and the like. As exemplified below, the CNS-reactive T-cells cross the blood brain barrier and undergo apoptosis at their target site of action, which in the case of EAE and MS is the CNS.

### II. Compositions and Administration

### Oligonucleotides and Nucleobase Oligomers

IAP antisense oligonucleotides or antisense IAP nucleobase oligomers reduce the amount of an IAP produced in a cell, allowing a cell normally expressing the IAP, to undergo apoptosis. This is accomplished by providing oligomers that specifically hybridize with one or more polypeptides encoding an IAP. The specific hybridization of the oligomers with an IAP polynucleotide (e.g. RNA, DNA) interferes with the normal function of that IAP polynucleotide, reducing the amount of IAP protein produced. This modulation of function of a target nucleic acid by compounds that specifically hybridize to the target is generally referred to as “antisense”.

Highly purified IAP antisense oligonucleotides useful in the present invention that are substantially free from other oligonucleotides and contaminants may be produced as described in U.S. Pat. No. 6,673,917. It is to be understood that while the IAP antisense oligonucleotide used in the present invention is a murine/human specific XIAP antisense oligonucleotide, specifically SEQ ID NO: 41, other human and therefore clinically relevant XIAP or HIAP1/2 antisense oligonucleotides are contemplated as illustrated in Tables 1 through 7 below.

Specific examples of useful IAP antisense oligonucleotides useful in practicing the methods of the present invention include SEQ ID NOs: 1-96 and 195-275.

At least two types of oligonucleotides induce the cleavage of RNA by RNase H: polydeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and compositions of the present invention may be used in conjunction with any technologies that may be developed, including covalently-closed multiple antisense (CMAS) oligonucleotides (Moon et al., Biochem J. 346:295-303, 2000; PCT Publication No. WO 00/61595), ribbon-type antisense (RiAS) oligonucleotides (Moon et al., J. Biol. Chem. 275:4647-4653, 2000; PCT Publication No. WO 00/61595), and large circular antisense oligonucleotides (U.S. Patent Application Publication No. US 2002/0168631 A1).

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; preferably open linear structures are used. Within the oligonucleotide structure, the phosphate groups are commonly referred to as forming the backbone of the oligonucleotide. The normal linkage or backbone of RNA and DNA is a phosphodiester linkage.

Specific examples of nucleobase oligomers useful in this invention include oligonucleotides containing modified backbones or non-natural internucleoside linkages. As defined in herein, nucleobase oligomers having modified backbones include those that retain a phosphorus atom in the backbone and those that do not have a phosphorus atom in the backbone. For the purposes of this invention, modified oligonucleotides that do not have a phosphorus atom in their internucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides are U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,521,063; 5,506,337; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,677,439; 5,698,685; and 6,365,577, each of which is herein incorporated by reference.

In other nucleobase oligomers, both the sugar and the internucleoside linkage, i.e., the backbone, are replaced with novel groups. The nucleobase units are maintained for hybridization with an IAP polypeptide. One such nucleobase oligomer, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

The nucleobase oligomers can have phosphorothioate backbones and nucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—. The oligonucleotides can also have morpholino backborie structures as described in U.S. Pat. No. 5,034,506.

Nucleobase oligomers may also contain one or more substituted sugar moieties. Nucleobase oligomers comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particular examples are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other nucleobase oligomers can include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Examples of modifications are 2′-O-methyl and 2′-methoxyethoxy (2′-O—CH₂CH₂OCH₃, also known as 2′-O-(2-methoxyethyl) or 2′-MOE). Another modification is 2′-dimethylaminooxyethoxy (i.e., O(CH₂)₂ON(CH₃)₂), also known as 2′-DMAOE. Other modifications include, 2′-aminopropoxy (2′-OCH₂CH₂CH₂NH₂) and 2′-fluoro (2′-F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil(pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pp. 858-859, Kroschwitz, J. I., Ed., John Wiley & Sons, 1990, those disclosed by English et al., Angewandte Chemie, International Edition, 1991, 30, 613, and those disclosed by Sanghvi, Y. S., Chapter 15, Antisense Research and Applications, pp. 289-302, Crooke, S. T. and Lebleu, B., Ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and O-6 substituted purines, including 2-aminopropyladenine, 5-propynyluracil and 5-propynylcytosine. 5-methylcytosine substitutions have been shown to increase-nucleic acid duplex stability by 0.6-1.2. degree. C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-1060, 1994), a thioether, e.g., hexyl-S-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates are U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes nucleobase oligomers that are chimeric compounds. “Chimeric” nucleobase oligomers are nucleobase oligomers, particularly oligonucleotides that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers may be formed as composite structures of two or more nucleobase oligomers as described above. Such nucleobase oligomers, when oligonucleotides, have also been referred to in the art as hybrids or gapmers. Representative United States patents that teach the preparation of such hybrid structures are U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with this invention may be conveniently and routinely made through the well-known technique of solid phase synthesis. Equipment for such synthesis is sold by several vendors, including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations are U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The nucleobase oligomers of the invention encompass any pharmaceutically acceptable salts, esters, or salts of such esters, or any other compound that, upon administration to a patient, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

As used herein the term “prodrug” is intended to mean a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention can be prepared as SATE [(S-acetyl-2-thioethyl) phosphate] derivatives according to the methods disclosed in PCT Publication Nos. WO 93/24510 or WO 94/26764.

As used herein the term “pharmaceutically acceptable salts” is intended to mean salts that retain the desired biological activity of the parent compound and do not impart undesired toxicological effects thereto. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., J. Pharma Sci., 66:1-19, 1977). The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Examples of acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides and other nucleobase oligomers, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine.

The present invention also includes pharmaceutical compositions and formulations that include the nucleobase oligomers of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral, or parenteral. Parenteral administration includes intravenous, intraarterial, subcutaneous, intraperitoneal, or intramuscular injection or infusion; or intracranial, e.g., intrathecal or intraventricular, administration.

### Locked Nucleic Acids (LNAs)

Locked nucleic acids (LNAs) are nucleobase oligomers that can be employed in the present invention. LNAs contain a 2′-O, 4′-C methylene bridge that restrict the flexibility of the ribofuranose ring of the nucleotide analog and locks it into the rigid bicyclic N-type conformation. LNAs show improved resistance to certain exo- and endonucleases and activate RNAse H, and can be incorporated into almost any nucleobase oligomer. Moreover, LNA-containing nucleobase oligomers can be prepared using standard phosphoramidite synthesis protocols. Additional details regarding LNAs can be found in PCT publication No. WO 99/14226 and U.S. patent application Publication No. US 2002/0094555 A1, each of which is hereby incorporated by reference.

### Arabinonucleic Acids (ANAs)

Arabinonucleic acids (ANAs) can also be employed in methods and reagents of the present invention. ANAs are nucleobase oligomers based on D-arabinose sugars instead of the natural D-2′-deoxyribose sugars. Underivatized ANA analogs have similar binding affinity for RNA as do phosphorothioates. When the arabinose sugar is derivatized with fluorine (2′ F-ANA), an enhancement in binding affinity results, and selective hydrolysis of bound RNA occurs efficiently in the resulting ANA/RNA and F-ANA/RNA duplexes. These analogs can be made stable in cellular media by a derivatization at their termini with simple L sugars. The use of ANAs in therapy is discussed, for example, in Damha et al., Nucleosides Nucleotides & Nucleic Acids 20: 429-440, 2001.

Uncomplexed oligonucleotides are capable on entering cells. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Ribozymes

Catalytic RNA molecules or ribozymes that include an antisense IAP sequence of the present invention can be used to inhibit expression of an IAP polynucleotide in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591, 1988, and U.S. patent application Publication No. 2003/0003469 A1, each of which is incorporated by reference.

Accordingly, the invention also features a catalytic RNA molecule that includes, in the binding arm, an antisense RNA having between eight and nineteen consecutive nucleobases corresponding to a sequence of any one of Tables 1, 2, 6, and 7 disclosed in US patent application Publication No. 2005/0119217 A1. The catalytic nucleic acid molecule is formed in a hammerhead or hairpin motif, but may also be formed in the motif of a hepatitis delta virus, group I intron or RNaseP RNA (in association with an RNA guide sequence) or Neurospora VS RNA. Examples of such hammerhead motifs are described by Rossi et al., AIDS Research and Human Retroviruses, 8:183, 1992. Example of hairpin motifs are described in U.S. Pat. Nos. 5,527,895; 5,856,188, and 6,221,661, and by Hampel and Tritz, Biochemistry, 28:4929, 1989, and Hampel et al., Nucleic Acids Research, 18: 299, 1990. An example of the hepatitis delta virus motif is described by Perrotta and Been, Biochemistry, 31:16, 1992. The RNaseP motif is described by Guerrier-Takada et al., Cell, 35:849, 1983. The Neurospora VS RNA ribozyme motif is described by Collins et al. (Saville and Collins, Cell 61:685-696, 1990; Saville and Collins, Proc. Natl. Acad. Sci. USA 88:8826-8830, 1991; Collins and Olive, Biochemistry 32:2795-2799, 1993). These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

### RNA Interference

The nucleobase oligomers of the present invention may be employed in double-stranded RNAs for RNA interference (RNAi)-mediated knock-down of IAP expression. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). In RNAi, gene silencing is typically triggered post-transcriptionally by the presence of double-stranded RNA (dsRNA) in a cell. This dsRNA is processed intracellularly into shorter pieces called small interfering RNAs (siRNAs). The introduction of siRNAs into cells either by transfection of dsRNAs or through expression of siRNAs using a plasmid-based expression system is increasingly being used to create loss-of-function phenotypes in mammalian cells.

In one aspect of the invention, double-stranded RNA (dsRNA) molecule is made that includes between eight and nineteen consecutive nucleobases of a nucleobase oligomer disclosed in US patent application Publication No. 2005/0119217 A1. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002; Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505, 2002, each of which is hereby incorporated by reference.

Small hairpin RNAs consist of a stem-loop structure with optional 3′ UU-overhangs. While there may be variation, stems can range from 21 to 31 bp (generally 25 to 29 bp), and the loops can range from 4 to 30 bp (generally 4 to 23 bp). For expression of shRNAs within cells, plasmid vectors containing the polymerase III H1-RNA, tRNA, or U6 promoter, a cloning site for the stem-looped RNA insert, and a 4-5-thymidine transcription termination signal can be employed. The polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

Accordingly, Tables 1 through 7 below illustrate the IAP antisense oligonucleotides and antisense IAP nucleobase oligomers which may be useful in practicing the methods of the present invention.

TABLE I

XIAP Antisense Oligonucleotides

| | | |
|---|---|---|
| | Position | |
| | in XIAP | Antisense oligonucleotide |
| SEQ ID NO: | sequence | sequence |

| | | |
|---|---|---|
| 1 | 2 | AAAATTCTAAGTACCTGCA |
| 2 | 21 | TCTAGAGGGTGGCTCAGGA |
| 3 | 44 | CAGATATATATGTAACACT |
| 4 | 78 | TGAGAGCCCTTTTTTTGTT |
| 5 | 110 | AGTATGAAATATTTCTGAT |
| 6 | 134 | ATTGGTTCCAATGTGTTCT |
| 7 | 160 | TTAGCAAAATATGTTTTAA |
| 8 | 185 | TGAATTAATTTTTAATATC |
| 9 | 238 | ATTCAAGGCATCAAAGTTG |
| 10 | 326 | GTCAAATCATTAATTAGGA |
| 11 | 370 | AATATGTAAACTGTGATGC |
| 12 | 411 | GCAGAATAAAACTAATAAT |
| 13 | 430 | GAAAGTAATATTTAAGCAG |
| 14 | 488 | TTACCACATCATTCAAGTC |
| 15 | 508 | CTAAATACTAGAGTTCGAC |
| 16 | 535 | ACACGACCGCTAAGAAACA |
| 17 | 561 | TATCCACTTATGACATAAA |
| 18 | 580 | GTTATAGGAGCTAACAAAT |
| 19 | 607 | AATGTGAAACACAAGCAAC |
| 20 | 638 | ACATTATATTAGGAAATCC |
| 21 | 653 | CTTGTCCACCTTTTCTAAA |
| 22 | 673 | ATCTTCTCTTGAAAATAGG |
| 23 | 694 | CCTTCAAAACTGTTAAAAG |
| 24 | 721 | ATGTCTGCAGGTACACAAG |
| 25 | 759 | ATCTATTAAACTCTTCTAC |
| 26 | 796 | ACAGGACTACCACTTGGAA |
| 27 | 815 | TGCCAGTGTTGATGCTGAA |
| 28 | 835 | GTATAAAGAAACCCTGCTC |
| 29 | 856 | CGCACGGTATCTCCTTCAC |
| 30 | 882 | CTACAGCTGCATGACAACT |
| 31 | 907 | GCTGAGTCTCCATATTGCC |
| 32 | 930 | ATACTTTCCTGTGTCTTCC |
| 33 | 950 | GATAAATCTGCAATTTGGG |
| 34 | 990 | TTGTAGACTGCGTGGCACT |
| 35 | 1010 | ACCATTCTGGATACCAGAA |
| 36 | 1029 | AGTTTTCAACTTTGTACTG |
| 37 | 1059 | ATGATCTCTGCTTCCCAGA |
| 38 | 1079 | AGATGGCCTGTCTAAGGCA |
| 39 | 1100 | AGTTCTCAAAAGATAGTCT |
| 40 | 1126 | GTGTCTGATATATCTACAA |
| 41 | 1137 | TCGGGTATATGGTGTCTGA |
| 42 | 1146 | CAGGGTTCCTCGGGTATAT |
| 43 | 1165 | GCTTCTTCACAATACATGG |
| 44 | 1192 | GGCCAGTTCTGAAAGGACT |
| 45 | 1225 | GCTAACTCTCTTGGGGTTA |
| 46 | 1246 | GTGTAGTAGAGTCCAGCAC |
| 47 | 1273 | AAGCACTGCACTTGGTCAC |
| 48 | 1294 | TTCAGTTTTCCACCACAAC |
| 49 | 1316 | ACGATCACAAGGTTCCCAA |
| 50 | 1337 | TCGCCTGTGTTCTGACCAG |
| 51 | 1370 | GCGGCCCAAAACAAAGAAG |
| 52 | 1393 | GATTCACTTCGAATATTAA |
| 53 | 1413 | TATCAGAACTCACAGCATC |
| 54 | 1441 | GGAAGATTTGTTGAATTTG |
| 55 | 1462 | TCTGCCATGGATGGATTTC |
| 56 | 1485 | AAGTAAAGATCCGTGCTTC |
| 57 | 1506 | CTGAGTATATCCATGTCCC |
| 58 | 1525 | GCAAGCTGCTCCTTGTTAA |
| 59 | 1546 | AAAGCATAAAATCCAGCTC |
| 60 | 1575 | GAAAGCACTTTACTTTATC |
| 61 | 1610 | ACTGGGCTTCCAATCAGTT |
| 62 | 1629 | GTTGTTCCCAAGGGTCTTC |
| 63 | 1650 | ACCCTGGATACCATTTAGC |
| 64 | 1669 | TGTTCTAACAGATATTTGC |
| 65 | 1688 | TATATATTCTTGTCCCTTC |
| 66 | 1696 | AGTTAAATGAATATTGTTT |
| 67 | 1725 | GACACTCCTCAAGTGAATG |
| 68 | 1745 | TTTCTCAGTAGTTCTTACC |
| 69 | 1759 | GTTAGTGATGGTGTTTTCT |
| 70 | 1782 | AGATGGTATCATCAATTCT |
| 71 | 1801 | TGTACCATAGGATTTTGGA |
| 72 | 1820 | CCCCATTCGTATAGCTTCT |
| 73 | 1849 | ATTATTTTCTTAATGTCCT |
| 74 | 1893 | CAAGTGATTTATAGTTGCT |
| 75 | 1913 | TAGATCTGCAACCAGAACC |
| 76 | 1945 | CATCTTGCATACTGTCTTT |
| 77 | 1997 | CCTTAGCTGCTCTTCAGTA |
| 78 | 2018 | AAGCTTCTCCTCTTGCAGG |
| 79 | 2044 | ATATTTCTATCCATACAGA |
| 80 | 2076 | CTAGATGTCCACAAGGAAC |
| 81 | 2096 | AGCACATTGTTTACAAGTG |
| 82 | 2123 | AGCACATGGGACACTTGTC |
| 83 | 2144 | CTTGAAAGTAATGACTGTG |
| 84 | 2182 | CCTACTATAGAGTTAGATT |
| 85 | 2215 | ATTCAATCAGGGTAATAAG |
| 86 | 2234 | AAGTCAGTTCACATCACAC |
| 87 | 2375 | CAGTAAAAAAAATGGATAA |
| 88 | 2428 | TTCAGTTATAGTATGATGC |
| 89 | 2471 | TACACTTAGAAATTAAATC |
| 90 | 2630 | TCTCTATCTTTCCACCAGC |
| 91 | 2667 | AGAATCCTAAAACACAACA |
| 92 | 2709 | ATTCGCACAAGTACGTGTT |
| 93 | 2785 | TGTCAGTACATGTTGGCTC |
| 94 | 2840 | ACATAGTGTTTTGCCACTT |
| 95 | 2861 | CTTTGATCTGGCTCAGACT |
| 96 | 2932 | GAAACCACATTTAACAGTT |

Antisense oligonucleotides against HIAP1, which are also useful in practicing methods of the present invention are SEQ ID NOs: 97 through 194 in Table 2.

TABLE 2

HIAP1 Antisense Oligonucleotides

| | | |
|---|---|---|
| | Position in | |
| SEQ ID | HIAP1 | Antisense oligonucleotide |
| NO: | sequence | sequence |

| | | |
|---|---|---|
| 97 | 1152 | TCATTTGAGCCTGGGAGGU |
| 98 | 1172 | CGGAGGCTGAGGCAGGAGA |
| 99 | 1207 | GGTGTGGTGGTACGCGCCT |
| 100 | 1664 | ACCCATGCACAAAACTACC |
| 101 | 1865 | AGAATGTGCCAGTAGGAGA |
| 102 | 2440 | TCTCACAGACGTTGGGCTT |
| 103 | 2469 | CCAGTGGTTTGCAAGCATG |
| 104 | 3695 | GAAATTTAGTGGCCAGGAA |
| 105 | 4013 | AGAAATACACAATTGCACC |
| 106 | 4032 | TACTGATACATTTTAAGGA |
| 107 | 4057 | TTCAACATGGAGATTCTAA |
| 108 | 4076 | ATTTCTATGCATTTAGAGT |
| 109 | 4121 | AATACTAGGCTGAAAAGCC |
| 110 | 4142 | GGCTTTGCTTTTATCAGTT |
| 111 | 4165 | TCTAGGGAGGTAGTTTTGT |
| 112 | 4189 | GGGAAGAAAAGGGACTAGC |
| 113 | 4212 | GTTCATAATGAAATGAATG |
| 114 | 4233 | ATAAGAATATGCTGTTTTC |
| 115 | 4265 | TTCAAACGTGTTGGCGCTT |
| 116 | 4283 | ATGACAAGTCGTATTTCAG |
| 117 | 4317 | AAGTGGAATACGTAGACAT |
| 118 | 4338 | AGACAGGAACCCCAGCAGG |
| 119 | 4357 | CGAGCAAGACTCCTTTCTG |
| 120 | 4376 | AGTGTAATAGAAACCAGCA |
| 121 | 4395 | TGACCTTGTCATTCACACC |
| 122 | 4426 | TTATCCAGCATCAGGCCAC |
| 123 | 4445 | ACTGTCTCCTCTTTTCCAG |
| 124 | 4464 | TTTTATGCTTTTCAGTAGG |
| 125 | 4489 | ACGAATCTGCAGCTAGGAT |
| 126 | 4517 | CAAGTTGTTAACGGAATTT |
| 127 | 4536 | TAGGCTGAGAGGTAGCTTC |
| 128 | 4555 | GTTACTGAAGAAGGAAAAG |
| 129 | 4574 | GAATGAGTGTGTGGAATGT |
| 130 | 4593 | TGTTTTCTGTACCCGGAAG |
| 131 | 4612 | GAGCCACGGAAATATCCAC |
| 132 | 4631 | TGATGGAGAGTTTGAATAA |
| 133 | 4656 | GATTTGCTCTGGAGTTTAC |
| 134 | 4670 | GGCAGAAAATTCTTGATTT |
| 135 | 4696 | GGACAGGGGTAGGAACTTC |
| 136 | 4714 | GCATTTTCGTTATTCATTG |
| 137 | 4733 | CTGAAAAGTAAGTAATCTG |
| 138 | 4759 | GGCGACAGAAAAGTCAATG |
| 139 | 4812 | CCACTCTGTCTCCAGGTCC |
| 140 | 4831 | CCACCACAGGCAAAGCAAG |
| 141 | 4855 | TTCGGTTCCCAATTGCTCA |
| 142 | 4874 | TTCTGACATAGCATTATCC |
| 143 | 4893 | TGGGAAAATGTCTCAGGTG |
| 144 | 4907 | TATAAATGGGCATTTGGGA |
| 145 | 4926 | TGTCTTGAAGCTGATTTTC |
| 146 | 4945 | GAAACTGTGTATCTTGAAG |
| 147 | 4964 | TGTCTGCATGCTCAGATTA |
| 148 | 4988 | GAATGTTTTAAAGCGGGCT |
| 149 | 5007 | CACTAGAGGGCCAGTTAAA |
| 150 | 5040 | CCGCACTTGCAAGCTGCTC |
| 151 | 5070 | CATCATCACTGTTACCCAC |
| 152 | 5095 | CCACCATCACAGCAAAAGC |
| 153 | 5117 | TCCAGATTCCCAACACCTG |
| 154 | 5130 | CCCATGGATCATCTCCAGA |
| 155 | 5149 | AACCACTTGGCATGTTGAA |
| 156 | 5168 | CAAGTACTCACACCTTGGA |
| 157 | 5187 | CCTGTCCTTTAATTCTTAT |
| 158 | 5206 | TGAACTTGACGGATGAACT |
| 159 | 5225 | TAGATGAGGGTAACTGGCT |
| 160 | 5244 | TGGATAGCAGCTGTTCAAG |
| 161 | 5271 | CATTTTCATCTCCTGGGCT |
| 162 | 529 | TGGATAATTGATGACTCTG |
| 163 | 5309 | GTCTTCTCCAGGTTCAAAA |
| 164 | 5337 | TATTCATCATGATTGCATC |
| 165 | 5366 | CATTTCCACGGCAGCATTA |
| 166 | 5367 | CCAGGCTTCTACTAAAGCC |
| 167 | 5416 | GCTAGGATTTTTCTCTGAA |
| 168 | 5435 | TCTATAATTCTCTCCAGTT |
| 169 | 5454 | ACACAAGATCATTGACTAG |
| 170 | 5473 | TCTGCATTGAGTAAGTCTA |
| 171 | 5492 | CTCTTCCCTTATTTCATCT |
| 172 | 5515 | TCCTCAGTTGCTCTTTCTC |
| 173 | 5560 | GCCATTCTATTCTTCCGGA |
| 174 | 5579 | AGTCAAATGTTGAAAAAGT |
| 175 | 5598 | CCAGGATTGGAATTACACA |
| 176 | 5622 | ATTCCGGCAGTTAGTAGAC |
| 177 | 5646 | TAACATCATGTTCTTGTTC |
| 178 | 5675 | GTCTGTGTCTTCTGTTTAA |
| 179 | 5684 | TTCTCTTGCTTGTAAAGAC |
| 180 | 5703 | GTAAAATCGTATCAATCAG |
| 181 | 5723 | GGCTGCAATATTTCCTTTT |
| 182 | 5742 | GAGAGTTTCTGAATACAGT |
| 183 | 5761 | ACAGCTTCAGCTTCTTGCA |
| 184 | 5780 | AAATAAATGCTCATATAAC |
| 185 | 5821 | GAAACATCTTCTGTGGGAA |
| 186 | 5841 | GTTCTTCCACTGGTAGATC |
| 187 | 5862 | CTTCTTGTAGTCTCCGCAA |
| 188 | 5890 | TTGTCCATACACACTTTAC |
| 189 | 6097 | AACCAAATTAGGATAAAAG |
| 190 | 6181 | ATGTTCATATGGTTTAGAT |
| 191 | 6306 | TAAGTTTTACTTCACTTAC |
| 192 | 6369 | ATGTTCCCGGTATTAGTAC |
| 193 | 6432 | GGGCTCAAGTAATTCTCTT |
| 194 | 6455 | GCCCAGGATGGATTCAAAC |

Note that in any of the foregoing nucleobase oligomers, or any other nucleobase oligomers described herein, each nucleobase may independently be a DNA residue or RNA residue, such as a 2′-O-methyl or 2′-O-methoxyelthyl RNA residue. The nucleobase sequence of SEQ ID NO: 3 may be, for example, 5′-CAGATATATATGTA ACACT-3′,5′-CAGATATATATGTAACACU-3′, or 5′-mCmAGATATATATGTAA CAmCmU-3′ (wherein mX represents a 2′-O-methyl X residue). Additional modified nucleobases are known in the art. The linkages may be phosphodiester (PO), phosphorothioate (PS), or methylphosphonate (MP) linkages, or may have a mixed backbone (MB). The backbone may be any suitable backbone that allows hybridization of the nucleobase oligomer to the target IAP polynucleotide. Exemplary backbones are described herein. In other embodiments, the nucleobase oligomers include acridine-protected linkages, cholesteryl or psoralen components, C5-propynyl pyrimidines, or C5-methylpyrimidines. Suitable modifications to the nucleobase oligomers of the invention include those described above, as well as those in U.S. Patent Application Publication No. US 2002/0128216 A1, hereby incorporated by reference.

Examples of nucleobase oligomers are provided in Table 3, below (wherein mX represents a 2′-O-methyl X RNA residue).

TABLE 3

SEQ ID
NO:

| | | |
|---|---|---|
| 2x2 MB PO | | |
| DE4 as | MGmGTATCTCCTTCACCAGmUmA | 195 |
| DE4 rev | MAmUGACCACTTCCTCTATmGmG | 196 |
| δBC5 as | MGmATACCAGAATTTmGmU | 197 |
| δBC5 rev | MUmGTTTAAGACCATmAmG | 198 |
| mG4 as | MGmCTGAGTCTCCATACTGmCmC | 199 |
| mG4 sm | MGmGCTCTCTGCCCACTGAmAmU | 200 |
| 3x3 MB PO | | |
| F3 as | MAmUmCTTCTCTTGAAAATmAmGmG | 201 |
| F3 scr | MCmAmGAGATTTCATTTAAmCmGmU | 202 |
| F3 mm | MAmUmCTTGACTTGATTATmAmGmG | 203 |
| F3 rev | MGmGmATAAAAGTTCTCTTmCmUmA | 204 |
| E4 as | MCmGmCACGGTATCTCCTTmCmAmC | 205 |
| E4 scr | MCmUmACGCTCGCCATCGTmUmCmA | 206 |
| E4 rev | MCmAmCTTCCTCTATGGCAmCmGmC | 207 |
| E4 mm | MCmGmCACCCTATCTGGTTmCmAmC | 208 |
| G4 as | MGmCmUGAGTCTCCATATTmGmCmC | 209 |
| G4 scr | MGmGmCTCTTTCGCCACTGmAmAmU | 210 |
| G4 rev | MCmCmGTTATACCTCTGAGmUmCmG | 211 |
| G4 mm | MGmCmUGACACTCCAATTTmGmCmC | 212 |
| C5 as | MAmCmCATTCTGGTAACCAmGmAmA | 213 |
| C5 scr | mUmGmCCCAAGAATACTAGmUmCmA | 214 |
| C5 mm | MAmCmCATAGTGGATTGCAmGmAmA | 215 |
| C5 rev | MAmAmGACCATAGGTCTTAmCmCmA | 216 |
| D7 as | mGmAmUTCACTTCTTCGAATATmUmAmA | 217 |
| D7 scr | MUmGmAAATGTAAATCATCmUmUmC | 218 |
| D7 mm | MGmAmUTCTGTTCGATAATmUmAmA | 219 |
| D7 rev | MAmAmUTATAAGCTTCACTmUmAmG | 220 |

Phosphorothioate

| | | |
|---|---|---|
| PS-G4 as | GCTGAGTCTCCATATTGCC | 221 |
| PS-G4 sm | GGCTCTTTGCCCACTGAAT | 222 |
| PS-C5 as | ACCATTCTGGATACCAGAA | 223 |
| PS-C5 rev | AAGACCATAGGTCTTACCA | 224 |
| PS-F3 as | ATCTTCTCTTGAAAATAGG | 225 |
| PS-F3 rev | GGATAAAAGTTCTCTTCTA | 226 |
| PS-DE4 as | GGTATCTCCTTCACCAGTA | 227 |
| PS-DE4 rev | ATGACCACTTCCTCTATGG | 228 |
| PS-BC5 as | TCTGGATACCAGAATTTGT | 229 |
| PS-BC5 rev | TGTTTAAGACCATAGGTCT | 230 |
| PS-AB6 as | GGGTTCCTCGGGTATATGG | 231 |
| PS-AB6 rs | GGTATATGGCGTCCTTGGG | 232 |
| PS-D7 as | GATTCACTTCGAATATTAA | 233 |
| PS-D7 rs | AATTATAACGTTCACTTAG | 234 |
| Penetratin | | |
| F3 as | ATCTTCTCTTGAAAATAGG | 235 |
| G4 as | GCTGAGTCTCCATATTGCC | 236 |
| D7 as | GATTCACTTCGAATATTAA | 237 |
| C5 cs | TGCCCAAGAATACTAGTCA | 238 |

4X4 MBO PS (phosphorothioate linkages throughout)

| | | |
|---|---|---|
| G4 as | mGmCmUmGAGTCTCCATATmUmGmCmC | 239 |
| G4 sm | mGmGmCmUCTTTGCCCACTmGmAmAmU | 240 |
| DE4 as | mGmGmUmATCTCCTTCACCmAmGmUmA | 241 |
| DE4 rev | mAmUmGmACCACTTCCTCTmAmUmGmG | 242 |
| E2 as | mGmAmAmAGTAATATTTAAmGmCmAmG | 243 |
| E2 rm | mGmAmGmCAATTTATAATGmAmAmAmG | 244 |
| H2G as | mAmCmCmGCTAAGAAACATmUmCmUmA | 245 |
| H2G rm | mAmUmCmUTACAAAGAATCmCmGmCmA | 246 |
| A3 as | mUmAmUmCCACTTATGACAmUmAmAmA | 247 |
| A3 rev | mAmAmAmUACAGTATTCACmCmUmAmU | 248 |
| FG8 as | mUmGmCmACCCTGGATACCmAmUmUmU | 249 |
| FG8 rm | mUmUmUmACCATAGGTCCCmAmGmCmU | 250 |
| mG4 as | mGmCmUmGAGTCTCCATACmUmGmCmC | 251 |
| mG4 sm | mGmGmCmUCTCTGCCCACTmGmAmAmU | 252 |
| F1 as | mAmUmUmGGTTCCAATGTGmUmUmCmU | 253 |
| F1 rev | mUmCmUmUGTGTAACCTTGmGmUmUmA | 254 |
| B4 as | mAmCmAmGGACTACCACTTmGmGmAmA | 255 |
| B4 rev | mAmAmGmGTTCACCATCAGmGmAmCmA | 256 |
| G6 as | mAmAmGmCACTGCACTTGGmUmCmAmC | 257 |
| G6 sm | mCmAmCmTGGTTGACCTCAmCmAmAmG | 258 |
| E12 as | mUmGmUmCAGTACATGTTGmGmCmUmC | 259 |
| E12 sm | mCmUmAmGGTTGTCCATGAmCmUmGmU | 260 |

Penetratin and its use in mediating entry of nucleobase oligomers into cells are described in PCT Patent Application No. FR 91/00444.

Table 4 illustrates a number of 2×2 PS/PO chimeric oligonucleotides, which are known to decrease XIAP mRNA levels and therefore may be useful in practicing the present invention.

TABLE 4

| | | | SEQ ID |
|---|---|---|---|
| Oligonucleotide | Sequence* | | NO: |
| F3 AS | ATCTTCTCTTGAAAATAGG | (PS) | 261 |
| F3 AS | AUCTTCTCTTGAAAATAGG | (2x2 | 262 |
| | | PS/PO) | |
| F3 RP | GGATAAAAGTTCTCTTCTA | (PS) | 263 |
| G4 AS | GCTGAGTCTCCATATTGCC | (PS) | 264 |
| G4 AS | GCTGAGTCTCCATATTGCC | (2x2 | 265 |
| | | PS/PO) | |
| G4 SC | GGCTCTTTGCCCACTGAAT | (PS) | 266 |
| C5 AS | ACCATTCTGGATACCAGAA | (PS) | 267 |
| C5 AS | ACCATTCTGGATACCAGAA | (2x2 | 268 |
| | | PS/PO) | |
| C5 RP | AAGACCATAGGTCTTACCA | (PS) | 269 |
| AB6 AS | GGGTTCCTCGGGTATATGG | (PS) | 270 |
| AB6 RP | GGTATATGGCGTCCTTGGG | (PS) | 271 |
| DE4 AS | GGTATCTCCTTCACCAGTA | (PS) | 272 |
| DE4 RP | ATGACCACTTCCTCTATGG | (PS) | 273 |
| D7 AS | GATTCACTTCGAATATTAA | (PS) | 274 |
| D7 RP | AATTATAACGTTCACTTAG | (PS) | 275 |

*Bold residues = DNA residues with phosphorothioate linkages, underlined residues = 2′-O-methyl RNA bases, plain type = phosphodiester DNA residues.

A number of HIAP1 antisense sequences, which are known to reduce HIAP1 mRNA levels, and which may be useful in practicing the methods of the present invention are illustrated in Table 5.

TABLE 5

| | |
|---|---|
| Nucleobase oligomer sequence | SEQ ID NO: |

| | |
|---|---|
| AGCAAGGACAAGCCCAGTC | 276 |
| TGTAAACCTGCTGCCCAGA | 277 |
| AGAAGTCGTTTTCCTCCTT | 278 |
| CCGAGATTAGACTAAGTCC | 279 |
| ACTTTTCCTTTATTTCCAC | 280 |
| TCCCAAACACAGGTACTAT | 281 |
| CATTCTCAGCGGTAACAGC | 282 |
| ACCATCATTCTCATCCTCA | 283 |
| AATGTAACCTTCAACCATC | 284 |
| TTTGTATTCATCACTGTC | 285 |
| TCACATCTCATTACCAAC | 286 |
| CCAGGTGGCAGGAGAAACA | 287 |
| TGCAGACTTCAATGCTTTG | 288 |
| TAAGCAAGTCACTGTGGCT | 289 |
| CTGAGTCGATAATACTAGC | 290 |
| ACTAGCCATTAGTAAAGAG | 291 |
| CAACAGCAGAGACCTTGTC | 292 |
| ATAGCATACCTTGAACCAG | 293 |
| CATCTGTAGGCTAAGATGG | 294 |
| AGTTACCAGATGCCATCTG | 295 |
| AATCTACTCTGATAGTGGA | 296 |
| GTTTCTGAAGCCAACATCA | 297 |
| TCAACTTATCACCTCCTGA | 298 |
| AAGAACTAACATTGTAGAG | 299 |
| GTAGACAACAGGTGCTGCA | 300 |
| ATGTCCTCTGTAATTATGG | 301 |
| TACTTGGCTAGAACATGGA | 302 |
| GAAGCAACTCAATGTTAAG | 303 |
| TTTGGTCTTTTGGACTCAG | 304 |
| CCATAGATCATCAGGAATA | 305 |
| CAGGACTGGCTAACACATC | 306 |
| TTTAATGGCAGGCATCTCC | 307 |
| TTAAGCCATCAGGATGCCA | 308 |
| GCTACAGAGTAAGCTGTGT | 309 |
| CTCTAGGGAGGTAGTTTTG | 310 |
| AAGAAAAGGGACTAGCCTT | 311 |
| CAGTTCACATGACAAGTCG | 312 |
| GACTCCTTTCTGAGACAGG | 313 |
| ATTCACACCAGTGTAATAG | 314 |
| CAGAAGCATTTGACCTTGT | 315 |
| CCAGCATCAGGCCACAACA | 316 |
| TTTCAGTAGGACTGTCTCC | 317 |
| TGCAGCTAGGATACAACTT | 318 |
| AGAGGTAGCTTCCAAGTTG | 319 |
| GAAGTAATGAGTGTGTGGA | 320 |
| GGATTTGATGGAGAGTTTG | 321 |
| GAACTTCTCATCAAGGCAG | 322 |
| AGGTCCTATGTAGTAAAAG | 323 |
| CAATTTTCCACCACAGGCA | 324 |
| CATTATCCTTCGGTTCCCA | 325 |
| CTCAGGTGTTCTGACATAG | 326 |
| GCTCAGATTAGAAACTGTG | 327 |
| CTGCATGTGTCTGCATGCT | 328 |
| TTAACTAGAACACTAGAGG | 329 |
| CATAATAAAAACCCGCACT | 330 |
| CACCATCACAGCAAAAGCA | 331 |
| CTCCAGATTCCCAACACCT | 332 |
| GGAAACCACTTGGCATGTT | 333 |
| GTTCAAGTAGATGAGGGTA | 334 |
| GATAATTGATGACTCTGCA | 335 |
| ATGGTCTTCTCCAGGTTCA | 336 |
| GCATTAATCACAGGGGTAT | 337 |
| TAAAGCCCATTTCCACGGC | 338 |
| TGTTTTACCAGGCTTCTAC | 339 |
| GATTTTTCTCTGAACTGTC | 340 |
| CTATAATTCTCTCCAGTTG | 341 |
| ACACAAGATCATTGACTAG | 342 |
| TCTGCATTGAGTAAGTCTA | 343 |
| TCTTTTTCCTCAGTTGCTC | 344 |
| GTGCCATTCTATTCTTCCG | 345 |
| GTAGACTATCCAGGATTGG | 346 |
| AGTTCTCTTGCTTGTAAAG | 347 |
| TCGTATCAATCAGTTCTCT | 348 |
| GCAGAGAGTTTCTGAATAC | 349 |
| ATGTCCTGTTGCACAAATA | 350 |
| CTGAAACATCTTCTGTGGG | 351 |
| TTTCTTCTTGTAGTCTCCG | 352 |
| CTTCTTTGTCCATACACAC | 353 |
| GGAATAAACACTATGGACA | 354 |
| CATACTACTAGATGACCAC | 355 |
| TGTACCCTTGATTGTACTC | 356 |
| GAAATGTACGAACTGTACC | 357 |
| GATGTTTTGGTTCTTCTTC | 358 |
| CTATCATTCTCTTAGTTTC | 359 |
| ACACCTGGCTTCATGTTCC | 360 |
| GACTACAGGCACATACCAC | 361 |
| TGCCTCAGCCTGGGACTAC | 362 |
| AGGATGGATTCAAACTCCT | 363 |
| GAGAAATGTGTCCCTGGTG | 364 |
| GCCACAACAGAAGCATTTG | 365 |

Suitable human HIAP2 for use as antisense oligomers are identified in Table 6.

TABLE 6

| Nucleobase oligomer | |
|---|---|
| sequence | SEQ ID NO: |
| TTCTGAAAACTCTTCAATG | 366 |
| CTTAGCATAAAGTATCAGT | 367 |
| CAAAAAAGTACTGCTTAGC | 368 |
| CAAGATAAAACTTGTCCTT | 369 |
| TATCAGTCATGTTGTAAAC | 370 |
| CTAAATAACCTGTTCATCA | 371 |
| AGCACACTTTTTACACTGC | 372 |
| ACCACTATTATTCTTGATC | 373 |
| TGTATTTGTTTCCATTTCC | 374 |
| ACTGTAAACTCTATCTTTG | 375 |
| CTTAAGTGGGCTAAATTAC | 376 |
| CCTTCATATGGTCACACTA | 377 |
| GGTTACAAGCTATGAAGCC | 378 |
| CTAAGCAACTATAGAATAC | 379 |
| TCCTTGATTTTTCACAGAG | 380 |
| ATACTAACTTAAAGCCCTG | 381 |
| GGGTTGTAGTAACTCTTTC | 382 |
| TAGAACACAACTCTTTGGG | 383 |
| CTCTGAATTTCCAAGATAC | 384 |
| TTTACTGGATTTATCTCAG | 385 |
| TGAGTAGGTGACAGTGCTG | 386 |
| GGAGGCAGTTTTGTGCATG | 387 |
| CTATCTTCCATTATACTCT | 388 |
| TTGTTTGTTGCTGTTTGTC | 389 |
| TCCTTTCTGAGACAGGCAC | 390 |
| ACCAGCACGAGCAAGACTC | 391 |
| ACCTTGTCATTCACACCAG | 392 |
| TCCAGTTATCCAGCATCAG | 393 |
| GCTTTTGAATAGGACTGTC | 394 |
| GAGATGTCTTCAACTGCTC | 395 |
| GGGGTTAGTCCTCGATGAA | 396 |
| TCATTGCATAACTGTAGGG | 397 |
| GCTCTTGCCAATTCTGATG | 398 |
| ACCCTATCTCCAGGTCCTA | 399 |
| ACAGGCAAAGCAGGCTACC | 400 |
| GTTCTGACATAGCATCATC | 401 |
| CTCAGAGTTTCTAGAGAAT | 402 |
| ATGTTCTCATTCGAGCTGC | 403 |
| TGAACTGGAACACTAGATG | 404 |
| GCTCAGGCTGAACTGGAAC | 405 |
| TTGACATCATCATTGCGAC | 406 |
| ACCATCACAACAAAAGCAT | 407 |
| CCACTTGGCATGTTCTACC | 408 |
| TCGTATCAAGAACTCACAC | 409 |
| GGTATCTGAAGTTGACAAC | 410 |
| TTTCTTCTCCAGTGGTATC | 411 |
| TTCTCCAGGTCCAAAATGA | 412 |
| ACAGCATCTTCTGAAGAAC | 413 |
| CACAGGTGTATTCATCATG | 414 |
| CCAGGTCTCTATTAAAGCC | 415 |
| TTCTCTCCAGTTGTCAGGA | 416 |
| GAAGTGCTGACACAATATC | 417 |
| TTTTCCTTCTCCTCCTCTC | 418 |
| CATCTGATGCCATTTCTTC | 419 |
| AGCCATTCTGTTCTTCCGA | 420 |
| CCAGGATAGGAAGCACACA | 421 |
| ATGGTATCAATCAGTTCTC | 422 |
| CCGCAGCATTTCCTTTAAC | 423 |
| CAGTTTTTGAAGATGTTGG | 424 |
| GTGACAGACCTGAAACATC | 425 |
| GGGCATTTTCTTAGAGAAG | 426 |
| AGTACCCTTGATTATACCC | 427 |
| GAAATGTACGAACAGTACC | 428 |
| TGAAAAACTCATAATTCCC | 429 |
| CCATCTTTTCAGAAACAAG | 430 |
| CTATAATTCTCTCCAGTTG | 431 |
| CTCCCTTAGGTACACATAC | 432 |
| ACAAGCAGTGACACTACTC | 433 |
| GTAACTCCTGAAATGATGC | 434 |
| CAACAAATCCAGTAACTCC | 435 |
| CACCATAACTCTGATGAAC | 436 |

Other antisense IAP nucleobase oligomers, including those described in Table 2 of U.S. Pat. No. 6,087,173 and also provided in Table 7 below.

TABLE 7

| | |
|---|---|
| Nucleobase oligomer sequence | SEQ ID NO: |

| | |
|---|---|
| TAGGACTTGTCCACCTTTTC | 437 |
| TTGAAAATAGGACTTGTCCA | 438 |
| TCTTCTCTTGAAAATAGGAC | 439 |
| CATCTTCTCTTGAAAATAGG | 440 |
| GTCATCTTCTCTTGAAAATA | 441 |
| AAGTCATCTTCTCTTGAAAA | 442 |
| AAAAGTCATCTTCTCTTGAA | 443 |
| TTAAAAGTCATCTTCTCTTG | 444 |
| TGTTAAAAGTCATCTTCTCT | 445 |
| ACTGTTAAAAGTCATCTTCT | 446 |
| AAACTGTTAAAAGTCATCTT | 447 |
| CAAAACTGTTAAAAGTCATC | 448 |
| TTCAAAACTGTTAAAAGTCA | 449 |
| GATGTCTGCAGGTACACAAG | 450 |
| TAGCAAAAGTTTTTAATCTA | 451 |
| GCATGACAACTAAAGCACCG | 452 |
| AATCTGCAATTTGGGGATAC | 453 |
| TTGTACTGACCATTCTGGAT | 454 |
| TCTGCATGTGTCTCAGATGG | 455 |
| ACAATACATGGCAGGGTTCC | 456 |
| TGCCTACTATAGAGTTAGAT | 457 |
| TAATGGAATTCAATCCTGAT | 458 |
| CAACTAAAACACTGCCATGT | 459 |
| TATGATGCTTCTTATTCTTA | 460 |
| ATTTGTTAAGCCTATCTGAA | 461 |
| TCCACCAGCATGGAACAATT | 462 |
| AGAAAATGGACAGAATCCTA | 463 |
| CTATCATTAAATACGCTTTC | 464 |
| TATTAACAACATACATACTT | 465 |
| GGTTAGGTTACTGATGTTAG | 466 |

Other sequences for antisense IAP nucleobase oligomers useful in the methods of the invention are described, for example, in U.S. Pat. Nos. 6,355,194; 6,165,788; 6,077,709; 5,958,772; 5,958,771; U.S. Patent Application Publication No. 2003/0125287 A1 and 2002/0137708; Carter et al., “Regulation and targeting of antiapoptotic XIAP in acute myeloid leukemia” (Leukemia advance online publication 11 Sep. 2003; doi:10.1038/sj.leu.2403113); and Bilim et al., Int. J. Cancer 103:29-37 (2003). Further examples of other IAP antisense oligonucleotides that are useful in practicing the methods of the invention include, but are not limited to, human and mouse XIAP (hILP, hILP1, MIHA, API3), NAIP (Birc 1), HIAP-1 (cIAP2, API2, MIHC, hITA), HIAP-2 (cIAP1, HIHB), survivin (TIAP, MIHD, API4), livin (KIAP, ML-IAP, cIAP3, HIAP3), and BRUCE, and are described in U.S. Pat. No. 6,156,535, the contents of which are hereby incorporated by reference in their entirety. Additional suitable IAP antisense oligonucleotides that are useful in practicing the present invention are described in U.S. Pat. No's.; 6,087,173; 6,784,291; 6,365,351; 6,365,577; United States published patent application No. US2004/0102395 A1; United States published patent application, No. US2005/0119217 A1, the contents of which are hereby incorporated by reference in their entirety. Also contemplated by the present invention are RNAi's to target the above IAPs antisense oligonucleotides. Examples of suitable RNAi's may be found in United States published patent application, No. US2005/0148535 A1, the contents of which is hereby incorporated by reference in its entirety.

The IAP antisense oligonucleotides of the present invention can be used to form pharmaceutical compositions. An IAP antisense oligonucleotide may be administered to the patient suffering from an autoimmune disease characterized by apoptosis resistant cells within a pharmaceutically acceptable diluent, carrier or excipient, in unit dosage form. Conventional pharmaceutical practice may be employed to provide suitable formulations or compositions to administer the antisense oligonucleotides to the subject. Administration of the compositions may begin prophylactically before the patient is symptomatic. The IAP antisense oligonucleotide may be either a murine IAP antisense oligonucleotide or a human IAP antisense oligonucleotide.

Compositions of the present invention may also include two or more IAP antisense oligonucleotides, each IAP antisense oligonucleotide being capable of sensitizing apoptosis-resistant cells to undergo apoptosis. The IAP antisense oligonucleotides may be administered in the form of a therapeutic composition with a pharmaceutically acceptable carrier or diluent. The two or more IAP antisense oligonucleotides may be administered consecutively or simultaneously as desired.

Compositions of the present invention may also be used as part of a combination therapy with existing MS therapeutics, such as β-interferon. An MS treatment regimen may include administering to the human patient, either sequentially or consecutively, the IAP antisense oligonucleotides and the β-interferon in the form of a therapeutic composition with a pharmaceutically acceptable carrier or diluent.

Another aspect of the present invention provides a method of treating CNS inflammatory autoimmune diseases, such as MS in humans, which are characterized by apoptosis-resistant T-cells. This method includes administering to the human patient suffering from the symptoms of MS one or more of the compositions of the present invention so that the symptoms of the disease are alleviated. Also contemplated is a prophylactic method of preventing the onset of multiple sclerosis in a human, and includes a prophylactic administration of an IAP antisense oligonucleotide to the human subject before the onset of the symptoms of MS.

Any appropriate route of administration may be employed, for example, administration may be parenteral, intravenous, intraarterial, subcutaneous, intramuscular, intracranial, intraorbital, ophthalmic, intraventricular, intracapsular, intraspinal, intracistemal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Incorporation of carrier compounds into compositions of the present invention are also contemplated. As used herein, the term “carrier compound” or “carrier” is intended to refer to an oligonucleotide, or analog thereof, which is inert (i.e., does not possess biological activity per se) but is recognized as an oligonucleotide by in vivo processes that otherwise reduce the bioavailability of an oligonucleotide having biological activity by, for example, degrading the biologically active oligonucleotide or promoting its removal from circulation. The co-administration of an oligonucleotide and a carrier compound, typically with an excess of the latter substance, can result in a substantial reduction of the amount of partially degraded oligonuceotide recovered in the liver, kidney or other extracirculatory reservoirs, presumably due to competition between the carrier compound and the oligonucleotide for a common receptor. For example, the recovery of a partially phosphorothioate oligonucleotide in hepatic tissue can be reduced when it is coadministered with polyinosinic acid, dextran sulfate, polycytidic acid or 4-acetamido-4,isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

In contrast to a carrier compound, a “pharmaceutical carrier”, “excipient” or “diluent” is a pharmaceutically acceptable solvent, suspending agent or any other pharmacologically inert vehicle for delivering one or more oligonucleotides to an animal. The excipient may be liquid or solid and is selected, with the planned manner of administration in mind, so as to provide for the desired bulk, consistency, etc., when combined with a oligonucleotide and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients suitable for non-parenteral administration which do not deleteriously react with oligonucleotides can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of oligonucleotides may include sterile and non-sterile aqueous solutions, non-aqueous solutions in common solvents such as alcohols, or solutions of the oligonucleotides in liquid or solid oil bases. The solutions may also contain buffers, diluents and other suitable additives. Pharmaceutically acceptable organic or inorganic excipients suitable for non-parenteral administration which do not deleteriously react with oligonucleotides can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, penetration enhancers, salts for influencing osmotic pressure, buffers, colorings, flavorings and/or aromatic substances and the like which do not deleteriously interact with the oligonucleotide(s) of the formulation. Aqueous suspensions may contain substances which increase the viscosity of the suspension including, for example, sodium carboxymethylcellulose, sorbitol and/or dextran. The suspension may also contain stabilizers. The penetration enhancers may include surfactants, fatty acids, bile salts, chelating agents, and non-chelating non-surfactants (Lee et al., Critical Reviews in Therapeutic Drug Carrier Systems, 1991, p. 92).

Methods well known in the art for making formulations are found, for example, in “Remington's Pharmaceutical Sciences.” Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for IAP antisense oligonucleotides include ethylene-vinyl acetate copolymer particles, osmotic pumps, implantable infusion systems, liposomes and emulsions. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human subjects in therapeutically effective amounts (e.g., amounts which prevent, eliminate, or reduce a pathological condition) to provide therapy for a disease or condition. The dosage of therapeutic agent to be administered is likely to depend on such variables as the type and extent of the disorder, the overall health status of the particular patient, the formulation of the compound excipients, and its route of administration.

For the treatment of MS, one advantage of the present invention is that the therapeutic IAP antisense oligonucleotide was administered peripherally and not into the CNS where it could exacerbate the disease. For other diseases, however, the IAP antisense oligonucleotide may be applied to the site of the needed apoptosis event (for example, by injection into the synovium). However, it may also be applied peripherally to tissue in the vicinity of the predicted apoptosis event or to a blood vessel supplying the cells predicted to require induced apoptosis.

The formulation of therapeutic compositions and their subsequent administration is believed to be within the skill of those in the art. Dosing is dependent on severity and responsiveness of the disease state to be treated, with the course of treatment lasting from several days to several months, or until a diminution of the disease state is achieved. Optimal dosing schedules can be calculated from measurements of drug accumulation in the body of the patient. Persons of ordinary skill can readily determine optimum dosages, dosing methodologies and repetition rates. Optimum dosages may vary depending on the relative potency, stability or bioavilability of individual oligonucleotides, and may be generally estimated based on EC₅₀ found to be effective in in vitro and in vivo animal models. In general, dosage is from between 0.1 mg and 100 mg per kg of body weight and may be given once or more daily, weekly, monthly or yearly to an adult in any pharmaceutically acceptable formulation. Persons of ordinary skill in the art can easily estimate repetition rates for dosing based on measured residence times and concentrations of the drug in bodily fluids or tissues.

MS is a chronic disease and continual therapy is contemplated to be within the scope of the present invention. Following successful treatment, it may be desirable to have the patient undergo maintenance therapy to prevent the recurrence of the disease state, wherein the XIAP antisense oligonucleotide is administered in maintenance doses, ranging from 0.1 mg and 100 mg per kg of body weight and may be given once or more daily, weekly, monthly or yearly.

In addition to MS, the present invention further contemplates methods of treating other autoimmune diseases in humans that are characterized by cells that are resistant to apoptosis. The other autoimmune diseases are Crohn's disease, psoriasis, rheumatoid arthritis and the like, and animal models of the diseases thereof. Non-human animal models of the aforesaid diseases typically include mice and primates. In the case of Crohn's disease, the T-cells' site of action is the intestinal mucosa. In the case of psoriasis, the cells which are resistant to apoptosis include keratinocytes, as well as T-cells. The keratinocytes are contacted with the XIAP antisense oligonucleotide so that the keratinocytes undergo apoptosis within the skin.

In the case of rheumatoid arthritis, the population of T-cells further includes a population of apoptosis-resistant synoviocytes. The synoviocytes are contacted with IAP antisense oligonucleotide so that the synoviocytes undergo apoptosis in the synovium.

### III Antisense Gene Therapy

Autoimmune disease therapy may be accomplished by direct administration of a therapeutic IAP antisense oligonucleotide to a T-cell that is expected to require induced apoptosis. The antisense oligonucleotide may be produced and isolated by any one of many standard techniques known to those skilled in the art. Administration of IAP antisense oligonucleotides to T-cells can be carried out by any of the methods for direct oligonucleotide administration.

Retroviral vectors, adenoviral vectors, lentivirus, adeno-associated viral vectors, or other viral vectors with the appropriate tropism for cells likely requiring enhanced apoptosis may be used as a gene transfer delivery system for a therapeutic antisense IAP gene construct. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., BioTechniques 7:980-990, 1989; Le Gal La Salle et al., Science 259:988-990, 1993; and Johnson, Chest 107:77 S-83S, 1995).

Retroviral vectors are particularly well developed and have been used in clinical settings (Rosenberg et al., N. Engl. J. Med 323:370, 1990; Anderson et al., U.S. Pat. No. 5,399,346). Non-viral approaches may also be employed for the introduction of therapeutic DNA into cells otherwise predicted to undergo induced apoptosis. For example, IAPs may be introduced into a cell by lipofection (Feigner et al., Proc. Natl. Acad. Sci. USA 84:7413, 1987; Ono et al., Neurosci. Lett. 117:259, 1990; Brigham et al., Am. J. Med. Sci. 298:278, 1989; Staubinger et al., Meth. Enz. 101:512, 1983), the penetratin system (Allinquant et al., J. Cell Biol. 128:919-927, 1995; Prochiantz, Curr. Opin. Neurobiol. 6:629-634, 1996), asialorosonucoid-polylysine conjugation (Wu et al., J. Biol. Chem. 263:14621,1988; Wu et al., J. Biol. Chem. 264:16985, 1989); or, less preferably microinjection under surgical conditions (Wolff et al., Science 247:1465, 1990).

In the therapeutic nucleic acid constructs described, nucleic acid expression can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in ovarian cells, breast tissue, neural cells, T-cells, or B cells may be used to direct expression. Enhancers include, without limitation, those that are characterized as tissue- or cell-specific in their expression. Alternatively, if a clone is used as a therapeutic construct, regulation may be mediated by the cognate regulatory sequences or, if desired, by regulatory sequences derived from a heterologous source, including any of the promoters or regulatory elements described above.

### IV. Assays

Specific examples of apoptosis assays are provided in the following references. Assays for apoptosis in lymphocytes are disclosed by Li et al. in “Induction of apoptosis in uninfected lymphocytes by HIV-1 Tat protein”, Science 268:429-431, 1995; Gibellini et al., in “Tat-expressing Jurkat cells show an increased resistance to different apoptotic stimuli, including acute human immunodeficiency virus-type 1 (HIV-1) infection”, Br. J. haematol. 89:24-33, 1995; Martin et al., in “HIV-1 infection of CD4⁺ T-cells in vitro. Differential induction of apoptosis in these cells”, J. Immunol. 152:330-342, 1994; Terai et al., in Apoptosis as a mechanism of cell death in cultured T lymphoblasts acutely infected with HIV-1”, J. Clin. Invest. 87:1710-1715, 1991; Dhein et al., in “Autocrine T-cell suicide mediated by APO-I/(Fas/CD95)”, Nature 373:438-441, 1995; Katsikis et al, in “Fas antigen stimulation induces marked apoptosis of T lymphocytes in human immunodeficiency virus-infected individuals”, J. Exp. Med. 1815:2029-2036, 1995; Westendorp et al., in “Sensitization of T-cells to CD95-mediated apoptosis by HIV-1 tat and gp120”, Nature 375:497, 1995; and DeRossi et al., Virology 198:234-244, 1994.

Therapeutic compounds for use in treating autoimmune diseases that are characterized by apoptosis resistant cells may be screened for using methods of the present invention. Specifically, the present invention contemplates a method of identifying a compound that sensitizes a cell to apoptosis stimuli. This method can include providing a cell, which is overexpressing an IAP gene. The cell would then be treated with a test compound and analyzed to determine whether IAP gene overexpression is decreased in the presence of the test compound. A decrease in IAP gene expression, measured by IAP protein levels, would indicate that the compound sensitizes the cell to apoptosis stimuli and causing it to undergo apoptosis at the site of autoimmune disease.

Other assays contemplated by the present invention include methods of identifying a compound that inhibits gene expression in an apoptosis-resistant cell. Typically, the method can include contacting an RNA message for an IAP protein with a test compound and then determining whether IAP gene expression is decreased in the presence of the test compound. A decrease in expression would indicate that compound may be capable of sensitizing the cell to apoptosis stimuli and causing it to undergo apoptosis at the site of autoimmune disease.

Similarly, the present invention also contemplates a method of identifying a compound that disrupts or inhibits IAP protein function in an apoptosis-resistant cell. In this assay an IAP protein would be contacted with a test compound and the IAP protein function would be analyzed to evaluate whether the function is disrupted or inhibited in the presence of the test compound. A disruption or inhibition would therefore indicate that the compound may be capable of sensitizing the cell to apoptosis stimuli and causing it to undergo apoptosis at the site of autoimmune disease.

A test compound, which decreases or inhibits IAP gene expression, is one that reduces the amount of target mRNA, protein encoded by such mRNA, by at least 5% relative to an untreated control. Methods for measuring both mRNA and protein levels are well known in the art. The test compound may disrupt mRNA, inhibit translation of mRNA to proteins, or inhibit transcription of IAP DNA into IAP mRNA. The test compound may disrupt the IAP protein function or inhibit IAP protein function. Decrease or inhibition of IAP gene expression or of IAP protein function by a test compound will be an indication that the compound will sensitize apoptosis resistant cells at their site of action. Test compounds contemplated by the present invention include any IAP antisense oligonucleotide, which causes the aforesaid effects.

One particularly useful aspect of the present invention would be to test a subject patient's suitability for receiving IAP antisense oligonucleotide therapy. The subject, who may be suffering from the symptoms of the autoimmune disease or who may be in remission would typically present themselves at a clinic or in a physician's office where a blood sample is drawn from the patient using a kit of the present invention described below. The blood sample would then be purified to isolate apoptosis-resistant cells. The sample may be treated with a number of test IAP antisense oligonucleotide or other agents or test compounds, followed by addition of apoptosis stimuli. The level of apoptosis would then be assayed using any one of the methods described above. Cells that have undergone apoptosis in sufficient number would indicate that the patient's suitability for treatment using the test compound.

Animal models for autoimmune disease may also provide in vivo assays for identifying compounds or IAP antisense oligonucleotides that sensitize apoptosis-resistant cells to apoptosis stimuli. Using the disease-specific animal models as described in the Examples below, the test compound may be peripherally administered to a mammal suffering from an induced autoimmune disease. In the case of the EAE model for human multiple sclerosis, a sample of cerebrospinal fluid or tissue may be removed and analyzed, after administration of a test compound, for increased cell apoptosis. The sample would be compared to a control animal and an increase in the level of apoptosis would be an indication that the test compound increases the sensitivity of the cell to apoptosis stimuli at its target site, specifically the brain tissue or the myelin. In the case of rheumatoid arthritis or psoriasis animal models, synovial fluid samples or skin samples may be removed and compared to control animals as per the EAE model.

Once a suitable IAP antisense oligonucleotide or test compound is identified using methods described above, the IAP antisense oligonucleotide or the test compound would be manufactured using processes known to those skilled in the art.

### V. Kits

The present invention also contemplates an article of manufacture in the form of a kit for use in testing a patient's suitability for treatment using a purified IAP antisense oligonucleotide described above. The kit would typically include, packaged together, a vessel or vessels, such as a vial, which contain purified apoptosis stimuli and a purified IAP antisense oligonucleotide in the same vial or separately in different vials, a sterile needle for drawing peripheral blood or other tissue sample, and instructions for using the kit. The kits can be manufactured according to the specific autoimmune disease for which a patient sample is to be taken. The instructions can describe the steps necessary to take appropriate blood or tissue samples from the subject patient, and how to mix the apoptosis stimuli, the olignonucleotide and blood or tissue sample.

### EXAMPLES

The present invention is further illustrated by the following non-limiting examples:

### Abbreviations

The following abbreviations are used throughout

### AS Antisense

CFA Complete Freund's adjuvant;

### Ci Curie

CNS Central nervous system;
DNA Deoxyribonucleic acid;
EAE Experimental allergic encephalomyelitis;
IAP Inhibitor of apoptosis;

### IP Intraperitoneal;

MOG Myelin oligodendrocycte protein;
mRNA Messenger ribonucleic acid;
PBS Phosphate buffer solution;
RNA Ribonucleic acid;
RP-HPLC Reverse phase high performance liquid chromatography;
XIAP X-linked inhibitor of apoptosis protein.

### I. Oligonucleotide Synthesis.

The ability of the antisense oligonucleotides to sensitize to apoptosis stimuli apoptosis resistant cells was tested using oligonucleotides as exemplary nucleobase oligomers. The oligonucleotides were synthesized by IDT (Integrated DNA Technologies, USA) as chimeric, second-generation oligonucleotides, consisting of a core of phosphodiester DNA residues flanked on either side by two 2′-O-methyl RNA residues with a phosphorothioate linkage between the flanking RNA residues. Appropriate controls such as scrambled or mismatch oligonucleotides were also synthesized.

The chimeric, or mixed-backbone (MBO), 19-mer antisense oligonucleotides was synthesized as 2×2 MBO oligonucleotides, composed of two flanking 2′-O-methyl RNA residues at either end with phosphorothioate linkages, and a central core of 15 phosphodiester DNA residues. Each final product was desalted by Sephadex G-25 chromatography (IDT Inc., Coralville, Iowa). This chimeric wingmer configuration, and mix of phosphorothioate and phosphodiester linkages (referred to as 2×2 PS/PO), provided adequate stability while also reducing non-specific toxicity associated with phosphorothioate residues. Fully phosphorothioated non-chimeric (DNA) antisense oligonucleotides for in vivo and in vitro studies were synthesized by Trilink Biotech and purified by RP-HPLC.

### II. EAE/Multiple Sclerosis

### Example 1

### Prophylactic Treatment with XIAP Antisense

Human/murine specific XIAP antisense SEQ ID NO: 41 and control oligonucleotides (SEQ ID NO: 468) and SEQ ID NO: 467 were dissolved in saline. Antisense (10 mg/kg) was administered IP from 5 days before immunization with MOG+CFA, 5 days per week, until 40 days post-immunization with MOG+CFA (5 days on, 2 days off).

EAE was elicited by subcutaneous (s.c.) immunization of C57BL6 mice (base of the tail, 2 sides, 50 mL/side) with an emulsion containing 100 micrograms per 50 uL of MOG35-55 peptide and 0.5 mg of Mycobacterium tuberculosis H35RA in freund's adjuvant. The adjuvant pertussis toxin (200 ng/mouse) was injected IP on the day of immunization and again 2 days later. The MOG+CFA s.c. immunization was repeated 7 days later, injecting into the flanks. Mice were monitored daily for body weight and clinical signs of EAE. The time of onset was about 14 days after first immunization. Symptoms include loss of tail tone, limb weakness, and clumsiness. Animals were assessed by a standard sequence of observation and simple tests of physical ability (whether hind limbs splayed when lifted by the tail, whether and how quickly the animal righted when overturned).

Mice were monitored daily for clinical signs of EAE that was scored as: 1) hook tail; 2) flaccid tail; 3) hind limb weakness and poor righting ability; 4) inability to right and one hind limb paralyzed; 5) both hind limbs paralyzed with orwithout forelimb paralysis and incontinence; and 6) moribund. All mice were kept in specific pathogen-free environment. Animal maintenance and all experimental protocols were in accordance with the Canadian Council for Animal Care guidelines and were approved by McGill University animal care committee.

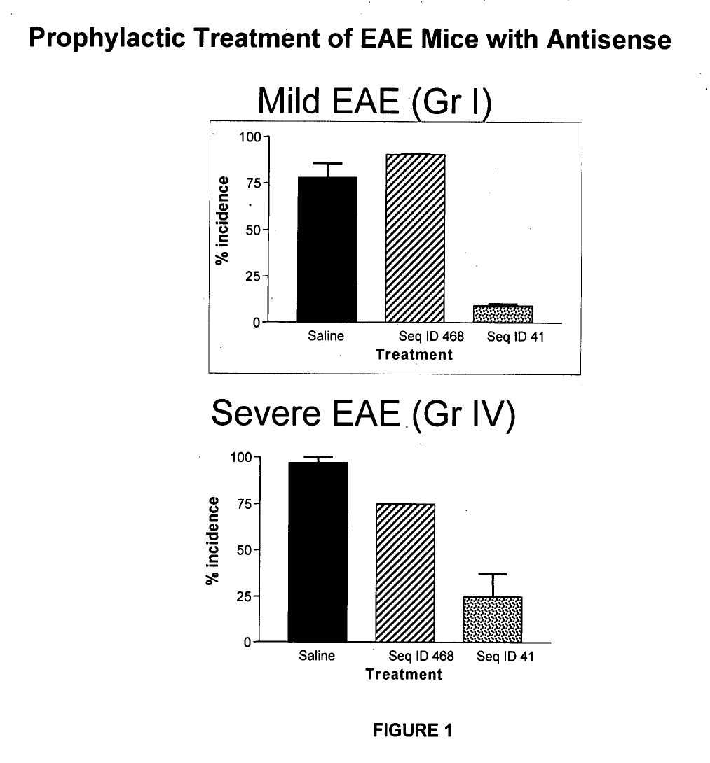

As illustrated in FIG. 1, the XIAP antisense (SEQ ID NO. 41), when administered prophylactically, reduces the number of mice that have either mild or severe disease. Only two mice out of 22 treated with XIAP antisense SEQ ID NO. 41 exhibited EAE symptoms. Conversely, 19 of 24 saline treated mice, 9 of 12 mice treated with control antisense SEQ ID NO: 467 and 10 of 11 mice treated with control antisense SEQ ID NO: 468 displayed symptoms of EAE.

### Example 2

### In Vitro Proliferation Assay of Lymph Node Cells

Mice were immunized for EAE as described in Example 1. A single cell suspension was prepared from the draining lymph nodes 14 days after the first immunization, and cells (4×10⁶/ml) were cultured for 4 days in 200 μl/well with or without 50 μg/ml MOG or control peptide (SIINFEKYL) in RPMI 1640 (Life Technologies, Burlington, Canada) supplemented with 10% FCS (Upstate Biotechnology, Lake Placid, N.Y.), 50 mM 2-ME (Sigma), 2 mM L-glutamine (Life Technologies), 100 U/ml penicillin (Life Technologies), and 100 μg/ml streptomycin (Life Technologies). Cultures were pulsed with 0.5 μCi of [³H]thymidine/well (ICN Biochemicals, Mississauga, Canada) during the last 18 h of incubation. [³H]thymidine uptake was measured as counts per minute.

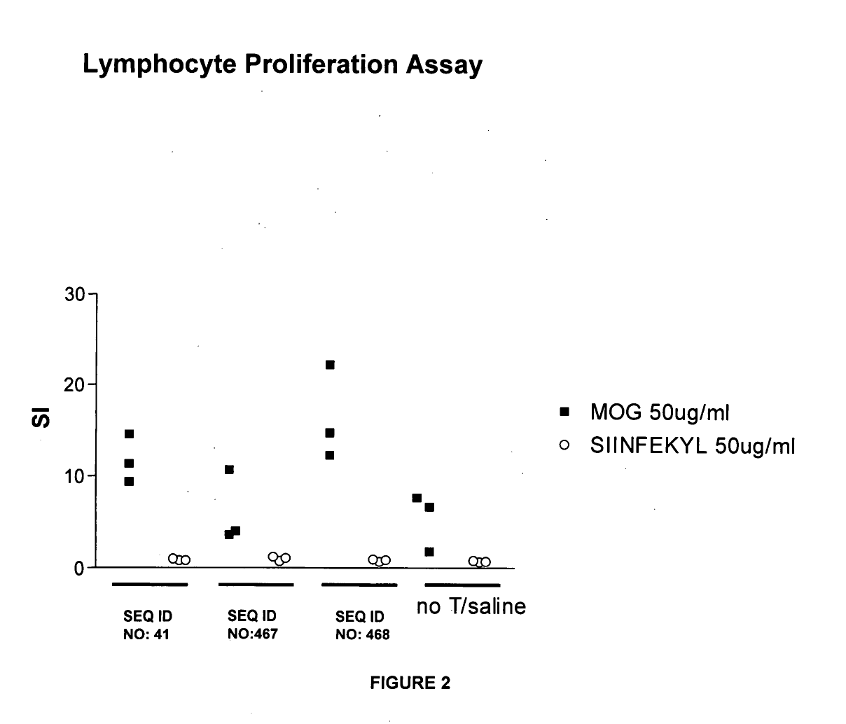

As illustrated in FIG. 2, XIAP antisense is not immunosuppressive. Lymph node T-cells were not affected by pre-treatment with oligonucleotides. This demonstrated that the mice mounted an appropriate immune reaction to MOG and thus the protection from EAE was not due to immune suppression.

### Example 3

### Flow Cytometric Analysis

EAE was induced as described in Example 1. After perfusion with ice-cold PBS, brains were removed, and spinal cords were dissected from the vertebral canal. Isolation of cells from the CNS was performed as follows. Animals were anaesthetized and perfused intracardially with ice cold PBS. Brain and spinal cord tissue was collected, mechanically dissociated and centrifuged at 400×g for 10 minutes at 4° C. The cell pellet was resuspended in 37% isotonic Percoll (Pharmacia Biotech, Mississauga) and centrifuged at 2800×g with no brake and moderate acceleration for 20 minutes at room temperature. Mononuclear cells were collected from the pellet and washed twice in 10% RPMI (Gibco, Burlington, Ontario). Cells were first incubated on ice for 30 min with 100 μg/ml normal rat Ig in 2.4G2 (anti-Fcγ RIIb/III) supernatant to block Fc receptors and avoid nonspecific staining, then double stained with PE-conjugated anti-Mac-1/CD11b (M1/70) and anti-CD45PE-CY5. Cells were analyzed using a FACScan (Becton Dickinson). Forward/side scatter gating were used to exclude dead cells.

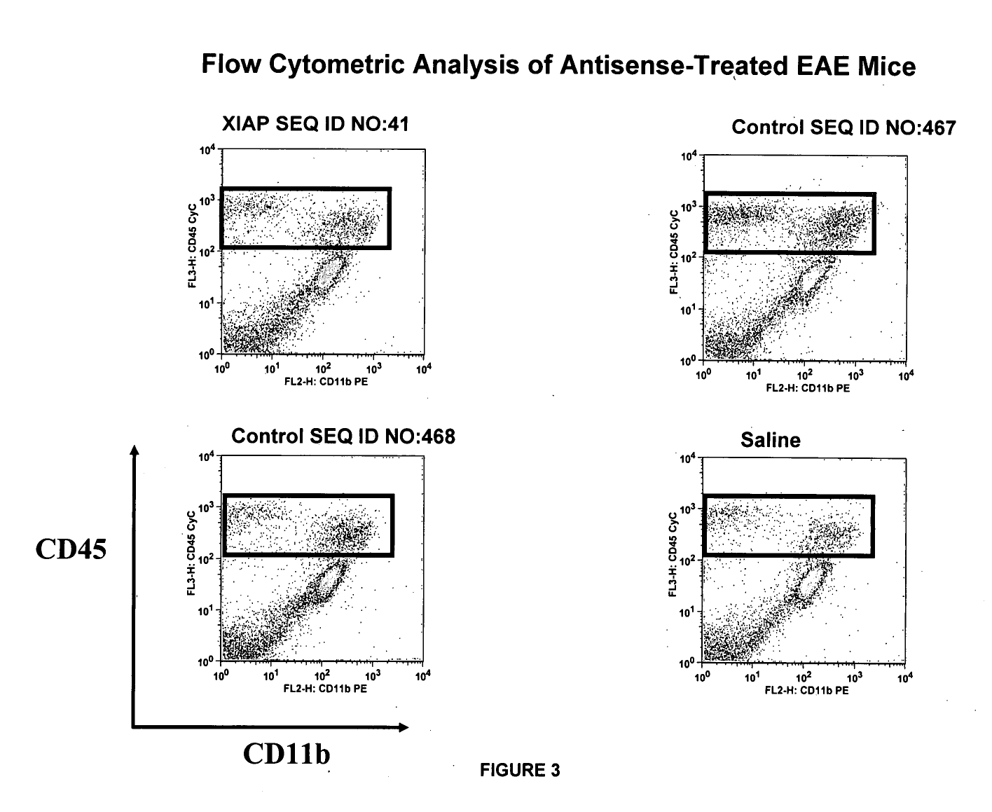

As illustrated in FIG. 3, mice without disease have CNS infiltrate. This indicates that mice protected by SEQ ID NO: 41 had CNS infiltrates similar, by this measure, to saline and control oligonucleotide treated mice which had EAE. SEQ ID NO: 41 blocks EAE despite the presence of infiltrate in the CNS.

### Example 4

### XIAP Antisense Therapeutically Treats EAE

EAE was elicited as described in Example 1. To investigate the effect of XIAP antisense on established disease mice were treated with antisense only after they first exhibited symptoms of EAE (Day 0). IP dosing at 10 mg/kg continued daily. Clinical scores were assessed daily.

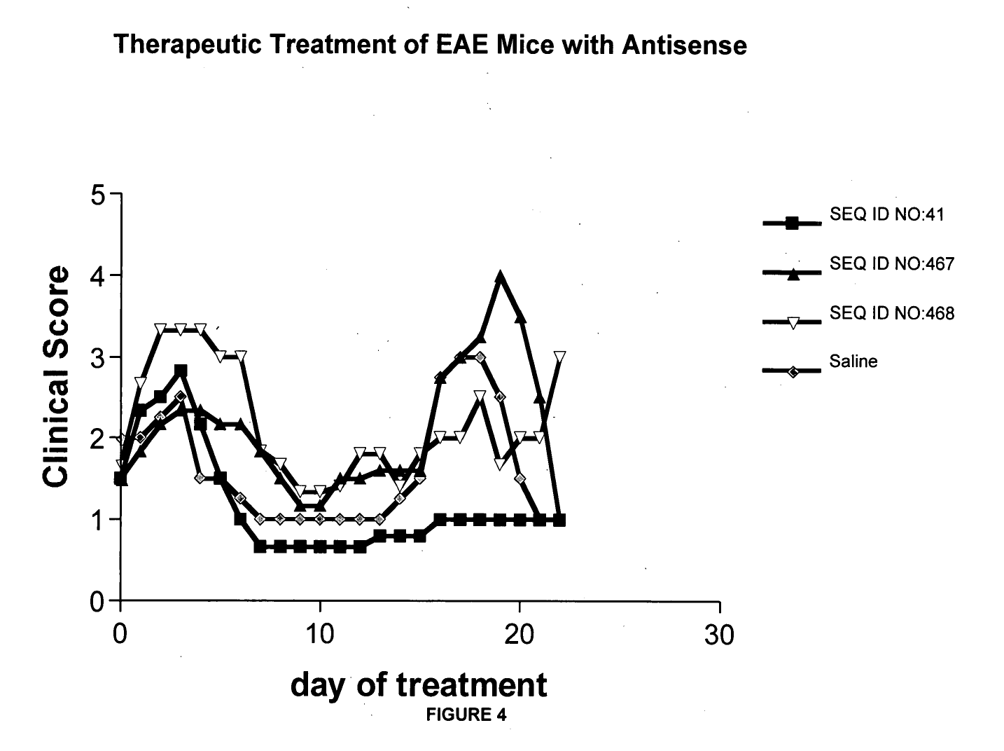

As illustrated in FIG. 4, XIAP antisense therapeutically treats EAE. C57BL6 mice were immunized for EAE. On the onset of EAE symptoms mice were dosed with 10 mg/mg oligonucleotides or saline daily. Control mice display 2 peaks of EAE symptoms separated by approximately 8 days. The occurrence of the second peak of disease was greatly diminished in SEQ ID NO: 41 treated mice.

### Example 5

### Reduced Microglial Activation in XIAP Antisense Protected Mice

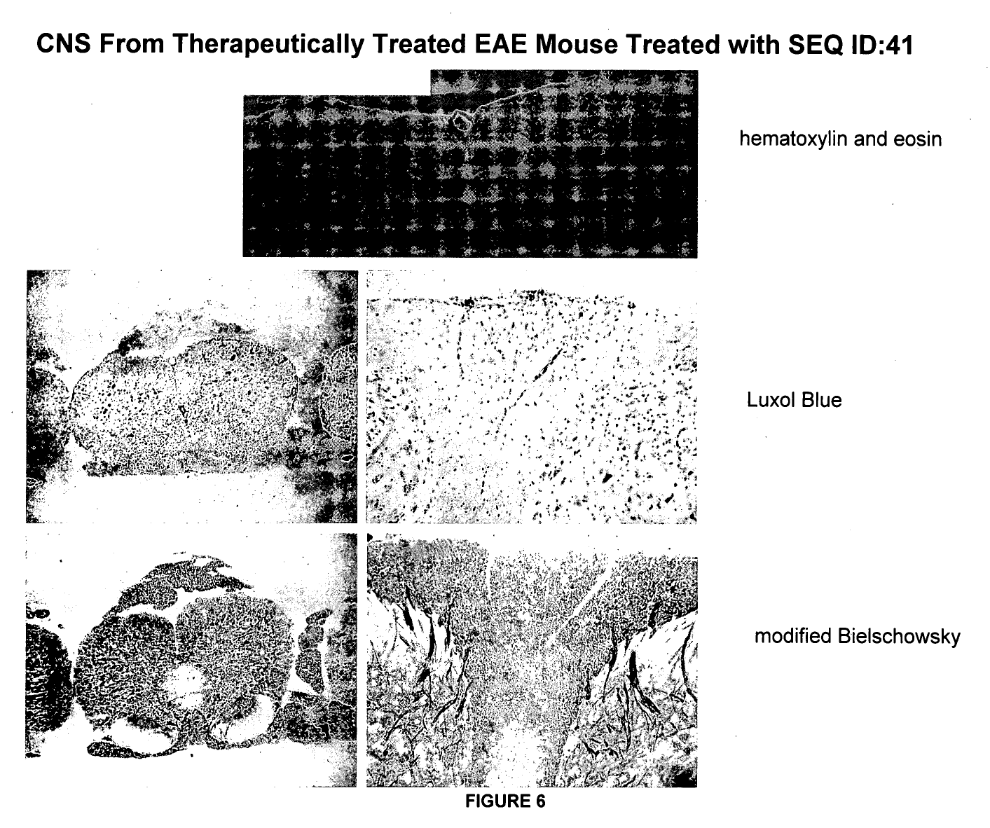

The microglial fraction of CNS infiltrates described above (FIG. 6) were analysed for activation. Cells were analysed by flow cytometry using PE-conjugated anti-Mac-1/CD11b (M1/70). Cells were analyzed using a FACScan (Becton Dickinson).

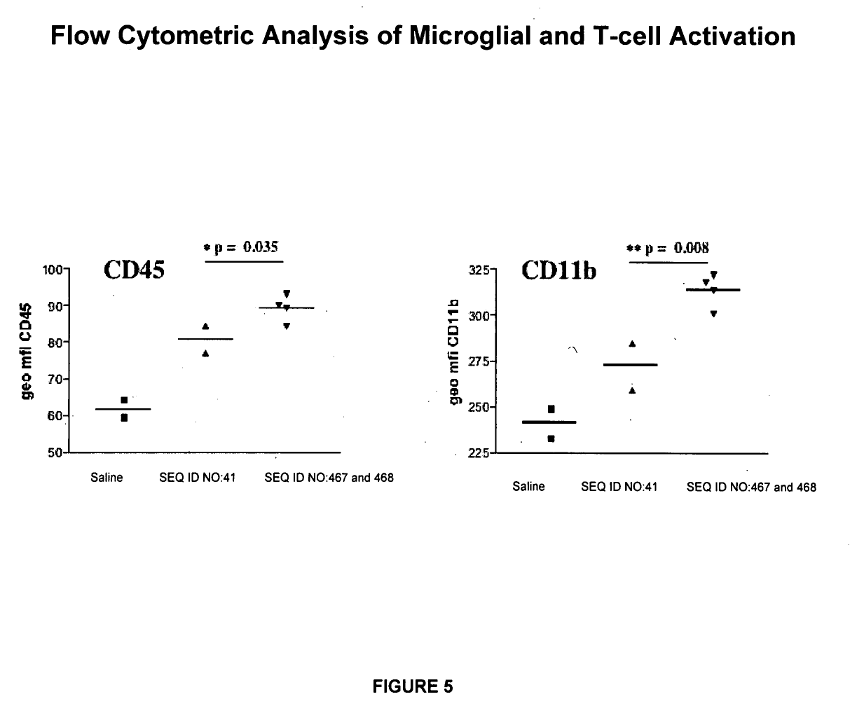

As illustrated in FIG. 5, there was reduced microglial activation in XIAP antisense protected mice. The microglial fraction of CNS infiltrates was assessed by flow cytometry. Reduced expression of CD45 and CD11b relative to non-protected control oligonucleotide treated mice (SEQ ID NO: 467) and SEQ ID NO: 468 demonstrated that XIAP antisense reduced the level of microglial activation in mice that were protected from EAE.

### Example 6

### CNS Histology of XIAP Antisense Treated Mice Following EAE

Mice were anesthetized with sodium pentobarbital (MT Pharmaceutical, Cambridge, Canada) and perfused intracardially through the left ventricle with ice-cold PBS followed by 10% buffered formalin. One-micron paraffin sections of CNS were stained with hematoxylin and eosin (H&E), Luxol Fast Blue or modified Bielschowsky stain.

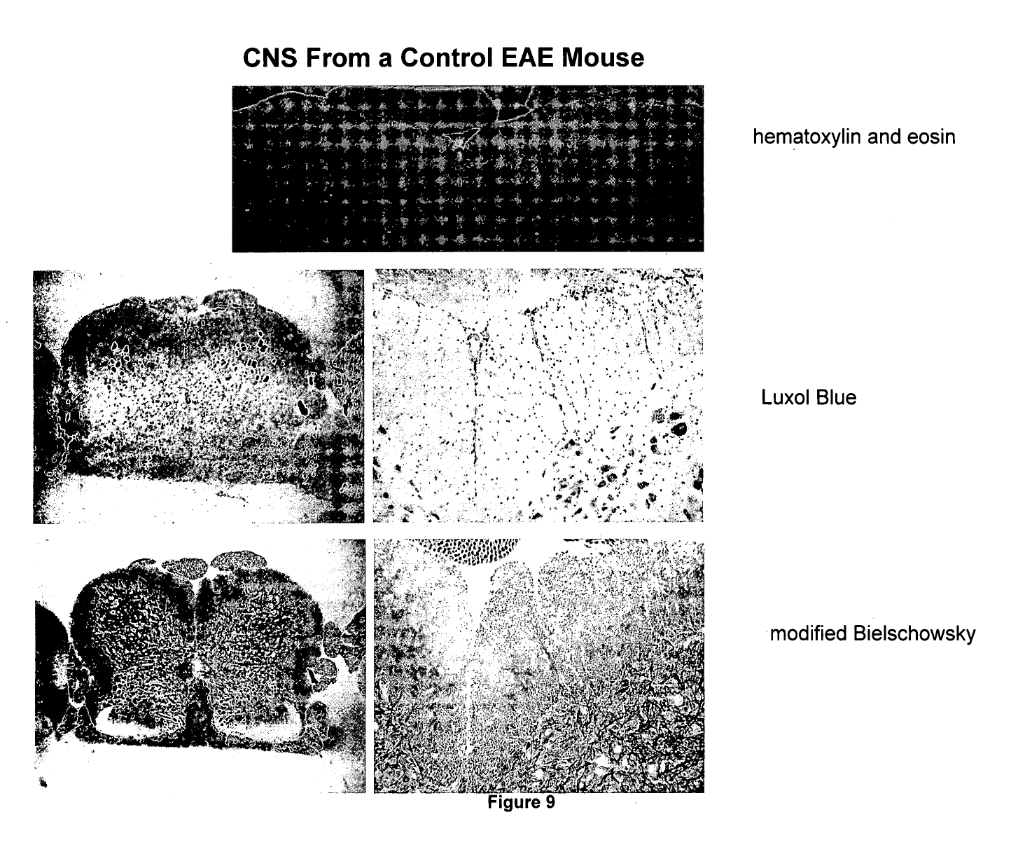

As illustrated in FIG. 6, histology of CNS from mice that were allowed to develop EAE and then treated with XIAP antisense reveals CNS infiltration despite attenuation of EAE by antisense treatment. H& E stain (top panel) showed lesions in white matter. Luxol Fast Blue stain revealed infiltration of the CNS (middle two panels, low and high magnification). Interestingly, unlike control EAE mice (FIG. 9), the pale blue myelin stain was not disrupted which suggested that there was little demyelination. Bielschowsky stain (lower two panels, low and high magnification) revealed some dispersion of the axonal stain which suggested that there was edema.

### Example 7

### Tunnel Analysis of CNS after Inhibition of EAE

EAE was elicited as described in Example 1. Animals were prophylactically treated with antisense and EAE was initiated. Mice were sacrificed when control mice showed frank disease. CNS was isolated from mice in which EAE was reduced by XIAP antisense treatment. Mice were anesthetized with sodium pentobarbital (MT Pharmaceutical, Cambridge, Canada) and perfused intracardially through the left ventricle with ice-cold PBS and then CNS was imbedded in OCT. Tunnel analysis (Roche Diagnostics) was performed on 10-μm cryostat sections as per the manufacturer's instructions. Tissues were co-stained with Hoechst to identify nuclei.

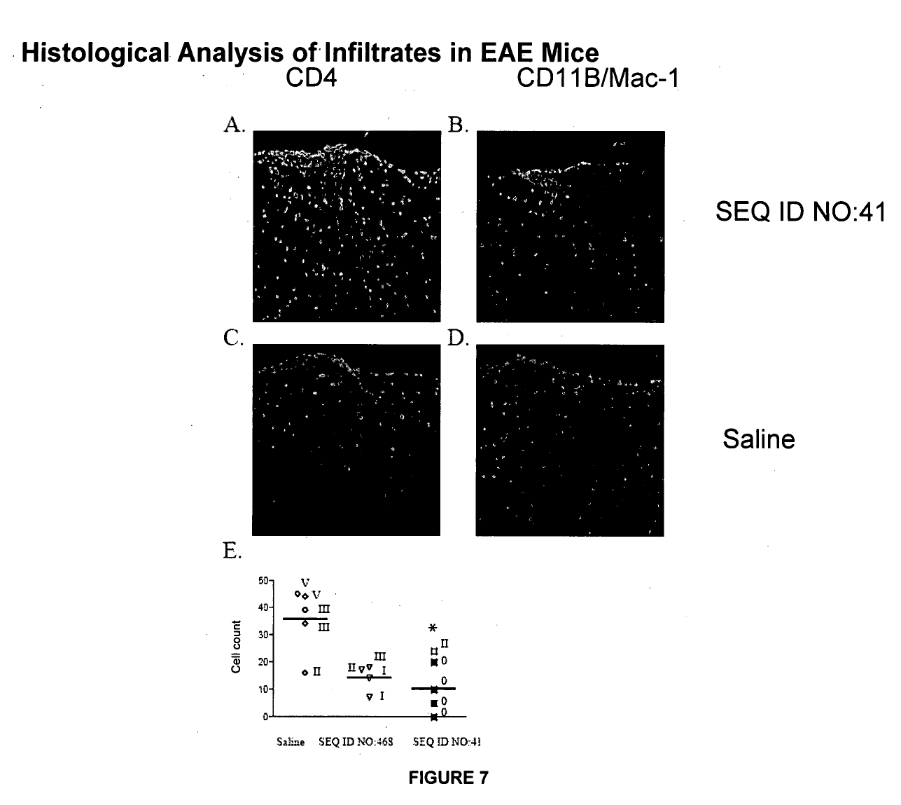

As illustrated in FIG. 7, CD4, CD11b/Mac-1 and Tunnel analysis was performed to determine the apoptotic status of EAE CNS and to characterize the infiltrating cells. Mice were treated with XIAP antisense or control compounds upon EAE onset. CNS tissue was obtained after 20 days of treatment. Infiltration of CD4+ ve T cells (red, A&C) and CD11b/Mac-1+ve cells (red, B &D) in prophylactically treated XIAP antisense (SEQ ID NO:41) (A&B) and saline (C &D) treated animals at peak disease. T cells (arrows) and macrophages (arrow heads) can be observed in the meningeal and perivascular areas of the white matter tracks of the spinal cord. Reduced areas of infiltrate were observed in control antisense (SEQ ID NO:468) treated animals. Semi-quantitation analysis of sections from saline (⋄), SEQ ID NO:468 (∇) and SEQ ID NO:41 (▪, □) treated animals show significant reduction in total infiltration with XIAP antisense (SEQ ID NO:41) treatment (E). Open symbols illustrate animals with clinical symptoms. 16 sections/animal throughout the length of the spinal cord were examined, results represent the total infiltrate. Statistical significance was analysed by one-way ANOVA * p<0.05). Interestingly, although a similar degree of infiltration was observed in control antisense (SEQ ID NO:468) and XIAP antisense (SEQ ID NO:41) treated animals, all control antisense animals had clinical symptoms whereas 4/5 XIAP antisense (SEQ ID NO:41) treated animals were asymptomatic (FIG. 7E, filled squares).

### Example 8

### Tunnel and Immunocytochemisty of EAE CNS

EAE was elicited as described in Example 1. XIAP antisense treatment started on the day that EAE symptoms were first observed and continued for 20 days. CNS was isolated from mice in which EAE was reduced by XIAP antisense (SEQ ID NO:41) treatment. Mice were anesthetized with sodium pentobarbital (MT Pharmaceutical, Cambridge, Canada) and perfused intracardially through the left ventricle with ice-cold PBS. CNS was isolated and then imbedded in OCT. Tunnel analysis (Green) (Roche Diagnostics), NeuN and Icam immunocytochemistry (Red) was performed on 10-μm cryostat sections. Sections were also stained with Hoechst (Blue).

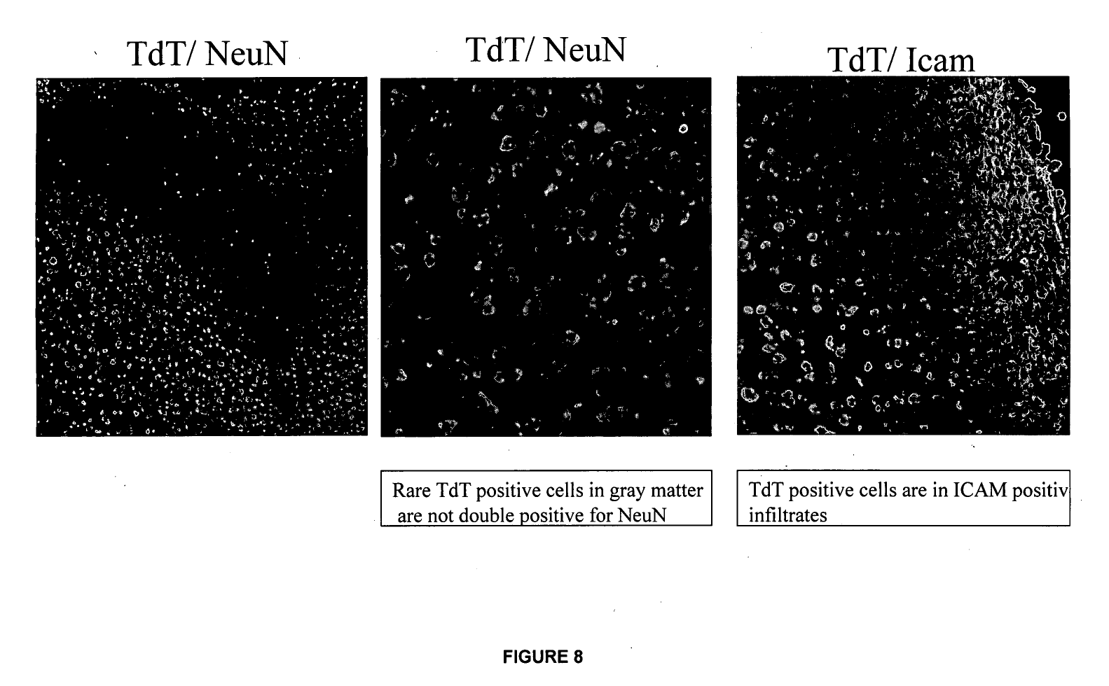

As illustrated in FIG. 8, CNS sections were stained for the neuronal marker NeuN and processed for tunel analysis. Apoptotic cells were not NeuN+ which indicated that they were not neurons. TdT positive cells were located in the white matter and were in ICAM positive infiltrates which suggested that the cells which underwent apoptosis were lymphocytes.

### Example 9

### CNS Histology of Control Antisense Treated Mice Following EAE

EAE was elicited as described in Example 1. Control antisense (SEQ ID NO: 467), 10 mg/kg) was administered daily for 20 days upon presentation of EAE symptoms. Mice were anesthetized with sodium pentobarbital (MT Pharmaceutical, Cambridge, Canada) and perfused intracardially through the left ventricle with ice-cold PBS followed by 10% buffered formalin. One-micron paraffin sections were stained with hematoxylin and eosin (H&E), Luxol Fast Blue or modified Bielschowsky stain.

As illustrated in FIG. 9, histology of CNS from mice that were allowed to develop EAE and then treated with XIAP antisense reveals robust CNS infiltration despite attenuation of EAE by antisense treatment. H& E stain (top panel) showed lesions in white matter. Luxol Fast Blue stain revealed robust infiltration of the CNS (middle two panels, low and high magnification). The pale blue myelin stain was disrupted which suggested that there was demyelination. Bielschowsky stain (lower two panels, low and high magnification) revealed some dispersion of the axonal stain.

### Example 10

### Increased Apoptosis of CD4+ ve T-Cells in the CNS of XIAP Antisense Treated Mice

EAE was elicited as described in Example 1. CNS was isolated and then imbedded in OCT. Tunnel analysis and CD4 immunocytochemistry was performed on 10-um cyrosections.

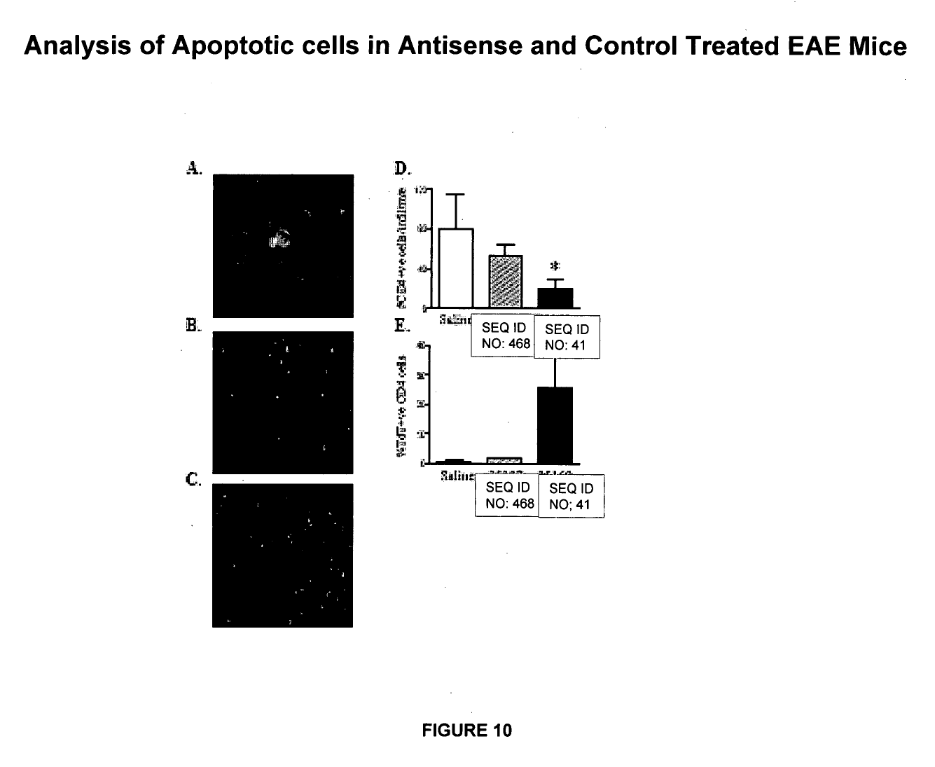

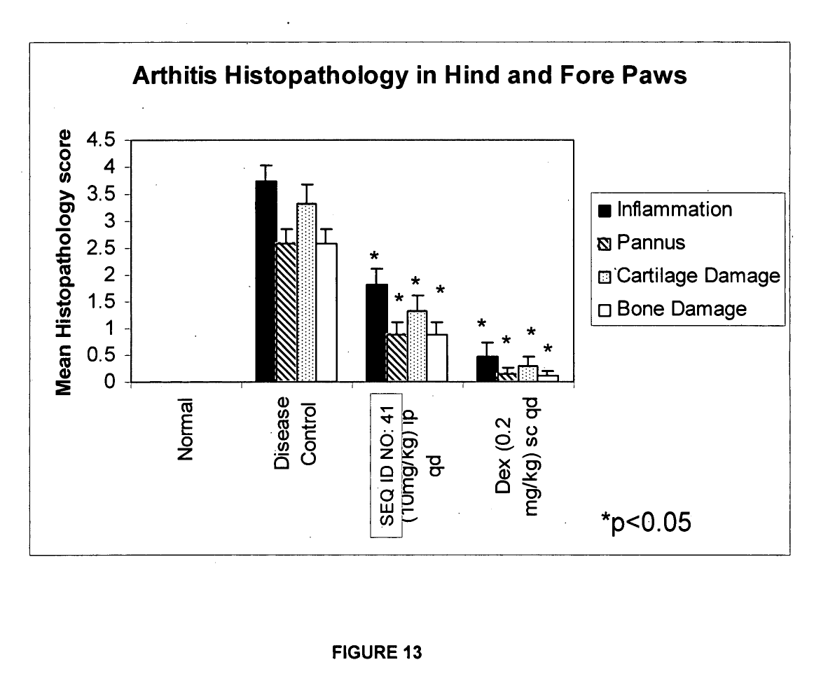

Dying CD4⁺ T-cells in CNS of SEQ ID NO:41 treated mice were identified by double immunoflourecence as membrane CD4⁺ (red), Hoescht nuclear (blue) and TUNEL+ve (green) cells (FIG. 10A). Infiltrating cells were analysed in prophylactically treated animals with EAE at peak disease. A reduction in the infiltrating T-cells was observed in mice treated with SEQ ID NO: 41 (FIG. 10B), as compared to saline treated animals (FIG. 10C). Enumeration of CD4⁺ cells/infiltrate illustrated a significant reduction in CD4⁺ T-cell numbers (FIG. 13D). The proportion of apoptotic CD4⁺ cells was significantly increased in SEQ ID NO: 41 treated animals (FIG. 10E). Results represent 24 infiltrates in 4-animals/treatment group. Statistical significance was analysed by one-way ANOVA * p<0.05.

### Example 11

### T-Cells from XIAP Antisense Treated Mice are More Susceptible to Apoptosis

EAE was elicited as described in Example 1. Mice were sacrificed when non-treated controls demonstrated frank disease. T-cells were purified from lymph nodes of saline and XIAP antisense treated mice and activated with anti-CD3 in vitro. CD4+ ve T-cells from XIAP antisense treated mice were significantly more susceptible to apoptosis than saline-treated controls.

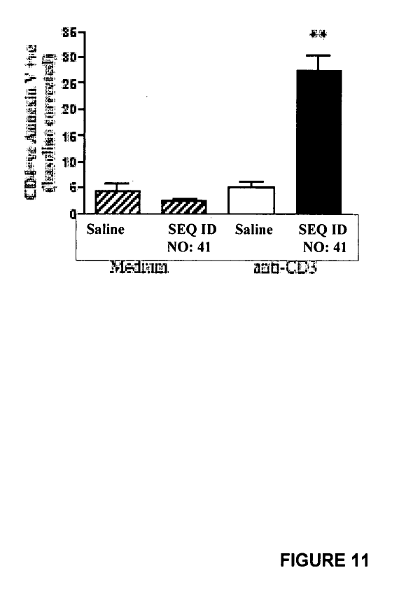

Purified T-cells were derived from lymph nodes (LN) of prophylactically XIAP antisense (SEQ ID NO:41) and saline treated animals at the time of peak disease in control treated animals. LN were stimulated for 24 with plate-bound anti-CD3, and activation induced cell death was quantitated by FACS analysis of CD4+ ve annexin V+ve cells. As illustrated in FIG. 11, significant exacerbation of CD4 T-cell death was observed in cells derived from SEQ ID NO: 41 treated animals in the presence of CD3 stimulation. Statistical significance was analyzed by one-way ANOVA ** p<0.001.

### III. Arthritis

A mouse model for human rheumatoid arthritis (RA), collagen-induced arthritis (CIA), was used to demonstrate efficacy of XIAP antisense treatment. CIA is an inflammatory disease that shares pathological and immunological features with human rheumatoid arthritis and is mediated by autoantibodies which bind to a particular region of type II collagen.

As exemplified below antisense IAP treatment protects mice from CIA. Without wishing to be bound by theory, we believe that the mechanisms by which IAP antisense oligonucleotides provide protection may include sensitizing T-cells to apoptosis at the joints. Additionally, IAP antisense may provide protection against CIA by sensitizing synovial fibroblasts or other cells involved in arthritis pathology to apoptosis.

### Example 12

### Prophylactic Treatment of Collagen-Induced Arthritis in Mice

Human/murine specific XIAP antisense SEQ ID NO: 41 was dissolved in saline. Mice were injected with type II collagen in Freund's complete adjuvant plus supplemental M. tuberculosis on days 0 and 15. Prophylactic treatment was initiated on day 12 by intraperitoneal injection of 10 mg/kg antisense. Control groups included untreated normal mice, untreated-collagen injected mice, and dexamethasone-treated collagen injected mice. Mice were scored for arthritis symptoms in fore and hind paws daily. Antisense treatment continued daily until day 26 when the mice were euthanized and tissues were collected for histological examination.

Clinical manifestations of arthritis were scored as follows: 0=normal, 1=1 hind or fore paw joint affected, 2=2 hind or fore paw joints affected, 3=3 hind or fore paw joints affected, 4=moderate erythema and moderate swelling or 4 digit joints affected, 5=severe erythema, and severe swelling of the entire paw, unable to flex digits.

Cartilage degeneration is scored none to severe (numerical values 0-5) for depth and area using the following criteria:0=no degeneration, normal, 1=minimal degeneration, chondrocyte and proteoglycan loss with or without fibrillation involving the superficial zone, 2=mild degeneration, chondrocyte and proteoglycan loss with or without fibrillation involving the upper ⅓, 3=moderate degeneration, chondrocyte and proteoglycan loss with fibrillation extending well into the midzone and generally affecting ½ of the total cartilage thickness, 4=marked degeneration, chondrocyte and proteoglycan loss with fibrillation extending well into the deep zone but without complete (to the tidemark) loss of matrix in all areas of the joint, 5=severe degeneration, matrix loss to the tidemark in majority of joints in the section

Inflammation, pannus and bone resorption are scored using the following criteria: 0=Normal, 1=Minimal infiltration of inflammatory cells in periarticular tissue, 2=Mild infiltration, 3=Moderate infiltration, 4=Marked infiltration, 5=Severe infiltration

Pannus was scored according to the following criteria: 0=Normal, 1=Minimal infilt. of pannus in cartilage, subchondral bone and periarticular tissue, 2=Mild infiltration with cartilage and/or bone destruction, some joints, 3=Moderate infiltration with cartilage and/or bone destruction, some joints, 4=Marked infiltration with cartilage and/or bone destruction, most joints, 5=Severe infiltration with cartilage and/or bone destruction, all joints

Bone Resorption was scored according to the following criteria: 0=Normal, 1=Minimal=small areas of resorption, not readily apparent on low magnification, rare osteoclasts, some joints, 2=Mild=more numerous areas of, not readily apparent on low magnification, osteoclasts more numerous, some joints, 3=Moderate=obvious resorption of medullary trabecular and cortical bone without full thickness defects in cortex, loss of some medullary trabeculae, lesion apparent on low magnification, osteoclasts more numerous, most joints, 4=Marked=Full thickness defects in cortical bone, often with distortion of profile of remaining cortical surface, marked loss of medullary bone, numerous osteoclasts, most joints, 5=Severe=Full thickness defects in cortical bone, often with distortion of profile of remaining cortical surface, marked loss of medullary bone, numerous osteoclasts, all joints

For each animal, the inflammation, pannus, cartilage damage and bone damage scores were determined for each of the 4 joints submitted. A sum total (all 6 joints) animal score was determined as well as sums and means for each of the individual parameters. Parameters for the various groups were compared to vehicle treated animals.

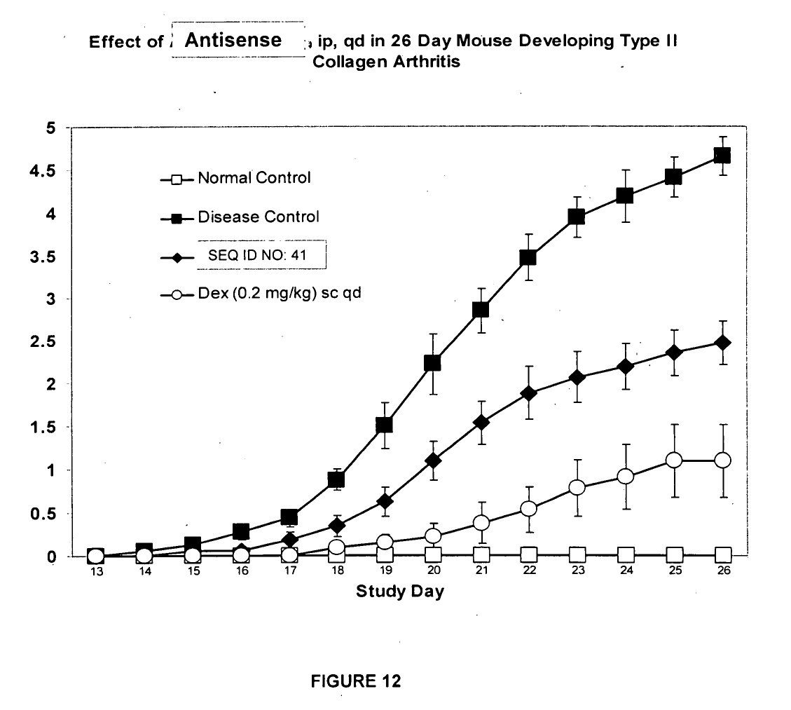

As illustrated in FIG. 12, the XIAP antisense when administered prophylactically, reduced the mean clinical arthritis score by more than 40%. Histopathological examination of the XIAP antisense treated mice showed a significantly significant reduction inflammation, pannus, cartilage and bone damage. XIAP antisense treatment also significantly reduced bone damage in the knee of CIA mice. The sum total of histopathology scores for all joints at the end of the experiment in the XIAP antisense treated group was reduced to 53% of the untreated disease controls.

As illustrated in FIG. 12 there was reduced collagen-induced arthritis in XIAP antisense treated mice. Specifically, mice were injected with type II collagen to induce arthritis. Daily treatments with either, saline (negative control), XIAP antisense or dexamethasone (positive control) were initiated 12 days later and continued for 14 days. Mice were observed daily and clinical manifestations of arthritis were scored as follows: 0=normal, 1=1 hind or fore paw joint affected, 2=2 hind or fore paw joints affected, 3=3 hind or fore paw joints affected, 4=moderate erythema and moderate swelling or 4 digit joints affected, 5=severe erythema, and severe swelling of the entire paw, unable to flex digits.

As illustrated in FIG. 13, there was reduced arthritis pathology in the paws of XIAP antisense-treated mice. Mice were injected with type II collagen to induce arthritis. Daily treatments with either, saline (negative control), XIAP antisense or dexamethasone (positive control) were initiated 12 days later and continued for 14 days. Mice were sacrificed and fore and hind paws processed for histology. Inflammation, pannus formation, cartilage and bone damage were scored fore each joint and summed.

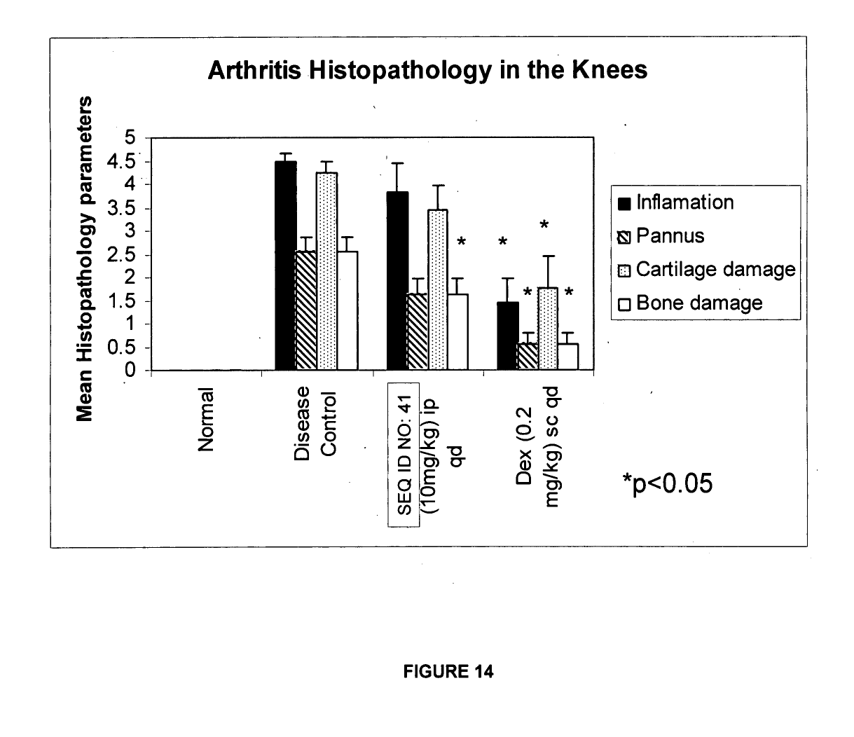

As illustrated in FIG. 14 there was reduced arthritis pathology in the knees of XIAP antisense-treated mice. Mice were injected with type II collagen to induce arthritis. Daily treatments with either, saline (negative control), XIAP antisense or dexamethasone (positive control) were initiated 12 days later and continued for 14 days. Mice were sacrificed and knees processed for histology. Inflammation, pannus formation, cartilage and bone damage were scored fore each joint and summed.

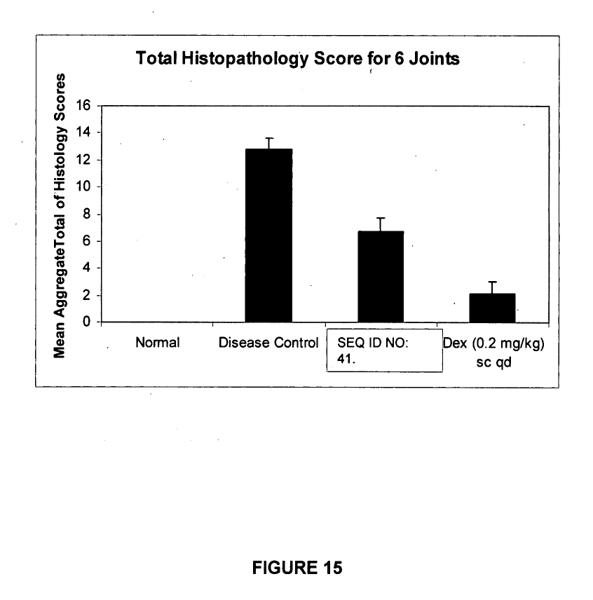

As illustrated in FIG. 15 there was reduced total arthritis pathology in of XIAP antisense-treated mice. Mice were injected with type II collagen to induce arthritis. Daily treatments with either, saline (negative control), XIAP antisense or dexamethasone (positive control) were initiated 12 days later and continued for 14 days. Inflammation, pannus formation, cartilage and bone damage were scored fore each joint and results for hind paws, fore paws and knees were summed.

### IV. Inflammatory Bowel Disease

A mouse model for human inflammatory bowel disease (IBD), including Crohn's disease, may be used to demonstrate efficacy of XIAP antisense treatment in autoimmune diseases afflicting the gut. Crohn's disease (CD) is a chronic inflammatory disorder of the gastrointestinal tract of unknown origin which may involve an excessive TH1 response. Hence, increased apoptosis of disease-related T-cells in the gastrointestinal tract could be of therapeutic value in the treatment of inflammatory bowel diseases such as Crohn's disease.

Experimental colitis can be induced by intrarectal instillation of 2,4,6-trinitrobenzene-sulfonic acid (TNBS) (2 mg/mouse in 50% ethanol of TNBS) in male Balb/C mice. Mice can be treated with IAP antisense immediately before induction of colitis and daily for 7 days. Body weight, presence of blood in the feces, as well as the presence and severity of diarrhea can be assessed. Colonic histopathology could also be assessed. Macroscopic damage, wall thickness (an index of edema formation) and myeloperoxydase activity (MPO; an index of granulocye infiltration) can be measured as indicators of antisense efficacy.

### V. Psoriasis

Animal models of human psoriasis may be used to demonstrate efficacy of XIAP antisense oligonucleotide treatment in autoimmune diseases afflicting the skin. Psoriasis is an immune-mediated disease in which chronic T-cell stimulation by antigen presenting cells occurs in the skin. Hence, increased apoptosis of disease-related T-cells in the skin can be of therapeutic value in the treatment of psoriasis. Orthotopic human skin graft models of psoriasis can be used to assess XIAP antisense efficacy in SCID mice. Psoriatic plaques can be transplanted or alternatively, pre-psoriatic skin can be used followed by injection of autologous activated T-cells. Animals bearing psoriatic plaque xenografts can be treated topically or systemically with XIAP antisense oligonucleotide. Clinical efficacy can be assessed by assessment of scaliness, induration, and erythema. Histologic examination of the psoriatic skin after treatment with XIAP antisense oligonucleotide for epidermal hyperplasia, grade of parakeratosis, T-cell number, and tunel positive T-cells would show reduced hyperplasia and parakeratosis and increased apoptosis of T-cells following antisense treatment.

### VI. Monitoring Patients During IAP Antisense Oligonucleotide Treatment of Autoimmune Diseases

### 1. Multiple Sclerosis

Drug efficacy in patients being treated with XIAP specific antisense for treatment of MS can be assessed using methods known to those skilled in the art. Patient assessments may include magnetic resonance imaging, and clinical measures such as the expanded disability status scale. Drug action could be measured by examining down regulation of target RNA or its cognate protein in the lymphocytes of treated patients.

### 2. Rheumatoid Arthritis

Drug efficacy in patients being treated with XIAP specific antisense oligonucletide for treating rheumatoid arthritis can be assessed using methods known to those skilled in the art. Antisense effects can be assessed by measuring disease activity by the Disease Activity Score in 28 joints (DAS28) and by measuring functional disability by the Health Assessment Questionnaire disability index. Drug action can be measured by examining down regulation of target RNA or its cognate protein in the lymphocytes of treated patients.

### 3. Crohn's Disease

Drug efficacy in patients being treated with XIAP antisense oligonucleotides to treat Crohn's disease can be assessed using methods known to those skilled in the art. Antisense effects can be assessed by assessing Crohn's Disease Activity Index (CDAI) scores in treated patients. Drug action can be measured by examining down regulation of target RNA or its cognate protein in the lymphocytes of treated patients.

### 4. Psoriasis

Drug efficacy in patients being treated with XIAP antisense oligonucleotides to treat psoriasis can be assessed by methods known to those skilled in the art. Antisense effects can be assessed in treated patients by assessing Dermatology Life Quality Index scores, ultrasound plaque thickness, plaque erythema, and analysis of immunohistochemical stains for immunocytes and proliferating cells. Drug action can be measured by examining down regulation of target RNA or its cognate protein in the lymphocytes of treated patients.

### OTHER EMBODIMENTS

From the foregoing description, it will be apparent to one of ordinary skill in the art that variations and modifications may be made to the invention described herein to adapt it to various usages and conditions. Such embodiments are also within the scope of the present invention.

All publications mentioned in this specification are hereby incorporated by reference.

While specific embodiments have been described, those skilled in the art will recognize many alterations that could be made within the spirit of the invention, which is defined solely according to the following claims:

What is claimed is:

1. A method of inducing an apoptosis-resistant cell to undergo apoptosis, the cell being associated with an autoimmune disease, the method comprising: sensitizing the apoptosis-resistant cell to apoptosis stimuli by treating the cell with an IAP antisense oligonucleotide, so that the cell undergoes apoptosis at a site of autoimmune disease.

2. The method, according to claim 1, in which the IAP antisense oligonucleotide comprises eight or more and thirty or less consecutive nucleobases in length.

3. The method, according to claim 1, in which the IAP antisense oligonucleotide is a XIAP antisense oligonucleotide.

4. The method, according to claim 1, in which the IAP antisense oligonucleotide is a HIAP1 antisense oligonucleotide.

5. The method, according to claim 1, in which the IAP antisense oligonucleotide is a HIAP2 antisense oligonucleotide.

6. The method, according to claim 1, in which the IAP antisense oligonucleotide comprises eight or more nucleobases of a sequence selected from the group consisting of: at least one of SEQ ID NOs: 1-466.

7. The method, according to claim 6, in which the IAP antisense oligonucleotide comprises eight or more nucleobases of a sequence selected from the group consisting of: at least one of SEQ ID NOs: 1-96, and 195-275.

8. The method, according to claim 7, in which the IAP antisense oligonucleotide consists of a sequence selected from the group consisting of: at least one of SEQ ID NOs: 31, 41, 47, 93, 195, 196, 197, 241, 245, 249, 270, and 272.

9. The method, according to claim 6, in which the IAP antisense oligonucleotide comprises eight or more nucleobases of a sequence selected from the group consisting of: at least one of SEQ ID NOs: 97-194, and 276-365.

10. The method, according to claim 6, in which the IAP antisense oligonucleotide comprises eight or more nucleobases of a sequence selected from the group consisting of: at least one of SEQ ID NOs: 366-436.

11. The method, according to claim 1, in which the apoptosis resistant cell is a T-cell, a synoviocyte, or a keratinocyte.

12. The method, according to claim 11, in which the apoptosis resistant cell is a T-cell.

13. The method, according to claim 12, in which the T-cell is a CD4+ T-cell.

14. The method, according to claim 1, in which the site of autoimmune disease is brain, myelin, intestinal mucosa, skin, or synovium.

15. The method, according to claim 14, in which the site of autoimmune disease is brain or myelin.

16. The method, according to claim 1, in which the autoimmune disease is EAE, multiple sclerosis, Crohn's disease, lupus erythematosus, rheumatoid arthritis, osteoarthritis, psoriasis, ulcerative colitis, type I diabetes, pancreatitis, asthma, idiopathic thrombocytopenia purpura, uveitis, Guillain-Barre syndrome or myasthenia gravis.

17. The method, according to claim 16, in which the autoimmune disease is multiple sclerosis.

18. A method of inducing apoptosis in an apoptosis-resistant cell, the cell being associated with an autoimmune disease, the method comprising: sensitizing the apoptosis-resistant cell to apoptosis stimuli by treating the cell with a XIAP antisense oligonucleotide comprising eight or more and thirty or less consecutive nucleobases in length, so that the cell undergoes apoptosis at a site of autoimmune disease.

19. The method, according to claim 18, in which the XIAP antisense oligonucleotide comprises eight or more nucleobases of a sequence selected from the group consisting of: at least one of SEQ ID NOs: 1-96, and 195-275.

20. The method, according to claim 19, in which the XIAP antisense oligonucleotide consist of a sequence selected from the group consisting of: at least one of SEQ ID NOs: 31, 41, 47, 93, 195, 196, 197, 241, 245, 249, 270, and 272.

21. A method of treating an autoimmune disease, the disease being characterized by apoptosis-resistant cells, the method comprising: administering to a mammalian subject in need thereof an IAP antisense oligonucleotide in a pharmaceutically acceptable carrier to sensitize the apoptosis-resistant cells to apoptosis stimuli, so that the cells undergo apoptosis at a site of autoimmune disease, thereby treating the disease.

22. The method, according to claim 21, in which the autoimmune disease is EAE, multiple sclerosis, Crohn's disease, lupus erythematosus, rheumatoid arthritis, osteoarthritis, psoriasis, ulcerative colitis, type I diabetes, pancreatitis, asthma, idiopathic thrombocytopenia purpura, uveitis, Guillain-Barre syndrome or myasthenia gravis.

23. The method, according to claim 22, in which the autoimmune disease is multiple sclerosis.

24. The method, according to claim 22, in which the autoimmune disease is rheumatoid arthritis.

25. The method, according to claim 21, in which the mammalian subject is a mouse, a human, a rat, a primate, or a guinea pig.

26. The method, according to claim 21, in which the mammalian subject is a human.

27. A method of treating a CNS inflammatory autoimmune disease characterized by apoptosis-resistant T-cells, the method comprising: administering to a mammalian subject a XIAP antisense oligonucleotide in a pharmaceutically acceptable carrier to sensitize the apoptosis-resistant T-cells to apoptosis stimuli, so that the T-cells undergo apoptosis at a site of CNS inflammatory autoimmune disease, thereby treating the disease.

28. The method, according to claim 27, in which the site of the CNS inflammatory autoimmune disease is brain or myelin.

29. The method, according to claim 27, in which the CNS inflammatory disease is multiple sclerosis.

30. A method of treating multiple sclerosis in a human, the method comprising: administering to the human a XIAP antisense oligonucleotide in a pharmaceutically acceptable carrier to sensitize apoptosis-resistarit T-cells to apoptosis stimuli, so that the T-cells undergo apoptosis at the brain or the myelin, thereby treating the multiple sclerosis.

31. A method of alleviating the symptoms of multiple sclerosis in a human, the method comprising: administering to the human a XIAP antisense oligonucleotide in a pharmaceutically acceptable carrier to sensitize apoptosis-resistant T-cells to apoptosis stimuli, so that the T-cells undergo apoptosis at the brain or the myelin, thereby alleviating the symptoms of multiple sclerosis.

32. A method of preventing the onset of multiple sclerosis in a human, the method comprising: administering to the human a XIAP antisense oligonucleotide in a pharmaceutically acceptable carrier to sensitize apoptosis-resistant T-celis to apoptosis stimuli, so that the T-cells undergo apoptosis at the brain or the myelin, thereby preventing the onset of multiple sclerosis in the human.

33. A method of predicting a patient's suitability for therapy, the method comprising:
a) isolating apoptosis-resistant cells from a blood sample taken from a patient suffering from an autoimmune disease characterized by apoptosis-resistant cells;
b) contacting the apoptosis-resistant cells with an IAP antisense oligonucleotide;
c) adding apoptosis stimuli to the contacted cells of step b); and
d) measuring apoptosis of the cells, apoptosed cells indicating that treatment with the IAP antisense oligonucleotide is suitable for the patient.

34. An in vivo assay for identifying a compound that sensitizes an apoptosis-resistant cell to apoptosis stimuli, the method comprising:
a) peripherally administering a test compound to a non-human mammal suffering from an autoimmune disease characterized by apoptosis-resistant cells; and
b) analyzing a sample of blood or tissue for increased cell apoptosis taken from the mammal, an increase in cell apoptosis being an indication that the test compound increases the sensitivity of the cell to apoptosis stimuli at a site of autoimmune disease.

35. An in vivo assay for identifying a compound that sensitizes an apoptosis-resistant cell to apoptosis stimuli, the method comprising:
a) administering a test compound to a site of autoimmune disease in a non-human mammal suffering from an autoimmune disease characterized by apoptosis-resistant cells; and
b) analyzing a sample of tissue taken from the site of autoimmune disease for increased cell apoptosis, an increase in cell apoptosis being an indication that the test compound increases the sensitivity of the cell to apoptosis stimuli at the site of the autoimmune disease.

36. A pharmaceutical composition, the composition comprising: an IAP antisense oligonucleotide in a pharmaceutically acceptable carrier, the oligonucleotide being in sufficient quantity to sensitize apoptosis-resistant cells to apoptosis stimuli so that the cells undergo apoptosis at a site of autoimmune disease, the disease being characterized by apoptosis-resistant cells.

37. The composition, according to claim 36, in which the IAP antisense oligonucleotide comprises eight or more and thirty or less consecutive nucleobases in length.

38. The composition, according to claim 36, in which the IAP antisense oligonucleotide is a XIAP antisense oligonucleotide.

39. The composition, according to claim 36, in which the IAP antisense oligonucleotide is a HIAP1 antisense oligonucleotide.

40. The composition, according to claim 36, in which the IAP antisense oligonucleotide is a HIAP2 antisense oligonucleotide.

41. The composition, according to claim 36, in which the IAP antisense oligonucleotide comprises eight or more nucleobases of a sequence selected from the group consisting of: at least one of SEQ ID NOs: 1-466.

42. The composition, according to claim 41, in which the IAP antisense oligonucleotide comprises eight or more nucleobases of a sequence selected from the group consisting of: at least one of SEQ ID NOs: 1-96, and 195-275.

43. The composition, according to claim 42, in which the IAP antisense oligonucleotide consist of a sequence selected from the group consisting of: at least one of SEQ ID NOs: 31, 41, 47, 93, 195, 196, 197, 241, 245, 249, 270, and 272.

44. The composition, according to claim 36, in which the IAP antisense oligonucleotide comprises eight or more nucleobases of a sequence selected from the group consisting of: at least one of SEQ ID NOs: 97-194, and 276-365.

46. The method, according to claim 36, in which the IAP antisense oligonucleotide comprises eight or more nucleobases of a sequence selected from the group consisting of: at least one of SEQ ID NOs: 366-436.

47. A kit comprising:
a) a vessel or vessels containing purified apoptosis stimuli and a purified IAP antisense oligonucleotide; and
b) instructions for drawing blood or a tissue sample from a subject and for mixing the blood with the oligonucleotide and the apoptosis stimuli.

48. A kit comprising:
a) a vessel or vessels containing purified apoptosis stimuli and a purified IAP antisense oligonucleotide;
b) a needle for drawing blood or a tissue sample; and
c) instructions for drawing blood or a tissue sample from a subject and for mixing the blood or the tissue sample with the oligonucleotide and the apoptosis stimuli.

49. An article of manufacture comprising:
a) a vial containing purified apoptosis stimuli and a purified IAP antisense oligonucleotide; or
b) packaged together, a first vial containing purified apoptosis stimuli and a second vial containing a purified IAP antisense oligonucleotide; and
c) instructions for drawing blood or a tissue sample from a subject and for mixing the blood or the tissue sample with the oligonucleotide and the apoptosis stimuli.

50. A method of inducing apoptosis, the method comprising: sensitizing to apoptosis stimuli at least one apoptosis-resistant cell in a population of cells, the apoptosis-resistant cell being contacted with an IAP antisense oligonucleotide so that the cell undergoes apoptosis at its target site.
